(19) United States
(12) Patent Application Publication
(10) Pub. No.: US 2019/0030114 A1
(43) Pub. Date: Jan. 31, 2019

(54) ISOFORM-SPECIFIC CALPAIN INHIBITORS, METHODS OF IDENTIFICATION, AND USES THEREOF

(71) Applicant: WESTERN UNIVERSITY OF HEALTH SCIENCES, Pomona, CA (US)

(72) Inventors: Michel BAUDRY, Pomona, CA (US); Xiaoning BI, Pomona, CA (US); Steve STANDLEY, Pomona, CA (US); Lyna LUO, Pomona, CA (US); Yubin WANG, Pomona, CA (US); Guoqi ZHU, Pomona, CA (US); Victor BRIZ, Pomona, CA (US)

(73) Assignee: WESTERN UNIVERSITY OF HEALTH SCIENCES, Pomona, CA (US)

(21) Appl. No.: 15/525,849

(22) PCT Filed: Nov. 11, 2015
(86) PCT No.: PCT/US15/60157

§ 371 Date: May 10, 2017

Related U.S. Application Data
(60) Provisional application No. 62/078,221, filed on Nov. 11, 2014.

(51) Int. Cl. A61K 38/06; A61P 25/08; A61P 27/06

(52) U.S. Cl. CPC A61K 38/06; A61P 27/06; A61P 25/08

(57) ABSTRACT

Molecules that selectively inhibit or stimulate the activity of isoforms of calpains are presented. Methods for screening and characterizing such molecules are also presented. Specific functions of calpain-1 calpain-2 in long term potentiation (LTP), learning and memory, neurodegeneration and diseases of synaptic dysfunction are characterized using novel calpain inhibitors, substrates and related methods. The compounds, compositions, and methods described herein are expected to be useful, for treating neurodegenerative diseases and other diseases of synaptic function, and for modulating cognition in patients in need thereof.

### CROSS-REFERENCE TO RELATED APPLICATIONS

This application claims priority to U.S. Application Ser. No. 62/078,221, filed 11 Nov. 2014, and includes its disclosure herein by reference.

### FIELD OF THE INVENTION

The invention relates to products, and methods of identifying products, that inhibit calpain-1 or calpain-2 function, and methods for specifically inhibiting calpain-1 or calpain-2 activation or activity, or for activating calpain-1, and to methods of treating and preventing diseases that are susceptible to treatment with molecules that, interfere with calpain-1 or calpain-2 function, or activate or augment calpain-1 activity.

### BACKGROUND

Generic calpain inhibitors and their use to treat diseases have been unsuccessful as therapeutics (Donkor, 2011, incorporated by reference). Herein, evidence is provided for the particular use of calpain-2 selective inhibitors, or separately calpain-1 inhibitors, or calpain-2 selective inhibitors and/or calpain-1 activators. The literature describes a few examples of inhibitors exhibiting higher selectivity for one calpain versus another (Li et al, 1996; Li et al, 1993, both incorporated by reference), but these disclosures acknowledge that the usefulness of a calpain-1 or calpain-2-selective inhibitor was unknown and required additional experimentation to determine if these compounds actually had therapeutic value.

Indeed, despite the lack of distinction between calpain-1 and calpain-2, the art has recognized a generalized need to develop selective inhibitors of calpain. Although generic calpain inhibitors (which includes more than 10 variants) have been used successfully as treatments in animal models of various diseases, none have progressed to clinical trials, in part due to lack of selectivity. Thus, there is a long-felt, but poorly understood need for more selective calpain inhibitors, and for a better understanding of the functions of calpain-1 and calpain-2. The evidence that compels such a conclusion is based on a few other distinctions among calpains that bear no insight into calpain-1 and calpain-2 distinguishability. For instance, calpain-10 gene (CAPN10) polymorphisms are associated with type 2 diabetes mellitus (T2DM); calpain-1 (μ-calpain), calpain-2 (m-calpain), calpain-3, and calpain-5 have also been linked to T2DM-associated metabolic pathways (Donkor, 2011).

Until this disclosure, it was not recognized that calpain-1 and calpain-2 are differentially linked to LTP, learning and memory, neurodegeneration, diseases of synaptic dysfunction, cell protective signaling cascades (calpain-1) and cell death cascades (calpain-2). Calpain-1 activation is linked to synaptic NMDA receptor stimulation, which accounts for its necessary role in LTP induction. It is also involved in neuroprotection elicited by prolonged synaptic NMDA receptor stimulation (see FIG. 1). On the other hand, calpain-2 is linked to extrasynaptic NMDA receptor stimulation and is involved in neurodegeneration (see FIG. 1). Calpain-2 is also activated by BDNF→ERK-mediated phosphorylation and limits the extent of LTP following theta-burst stimulation (TBS). Thus a selective calpain-2 inhibitor can be both neuroprotective and a cognitive enhancer.

Significant improvements in selectivity have been made in calpains versus cysteine proteinases such as cathepsins (Cuerrier et al, 2007, incorporated by reference) or other proteinases (Sorimachi et al, 2012, incorporated by reference). While the literature has described inhibitors with some degree of selectivity for calpain-2 and not calpain-1, or vice versa, none have been created with the benefit of a specific substrate until now. Calpain inhibitor IV (carboxybenzyl-Leu-Leu-Tyr-CH2-F) shows some selectivity for calpain-1 but the effect appears to vary with cell-type (Powers et al, 2002, incorporated by reference). While a complete rendering of the structural elements contributing to PTEN specificity may be difficult to capture in a small molecule, a full accounting of the specificity will provide a solid structural basis for designing a highly specific inhibitor of calpain-2.

### SUMMARY OF THE INVENTION

Described herein, are compositions and methods related to isoform-selective calpain inhibitors. Isoform-selective inhibitors are directed at either calpain-1 or calpain-2, and thus, selectively reduce the activity of one isoform in comparison to the other. Selective inhibitors of calpain-1 or calpain-2 may inhibit catalytic activity, reduce expression, selectively degrade, inhibit or hasten chemical modification, or affect protein interactions between calpain-1 or calpain-2 and one or more of its interacting proteins. Selective inhibitors of calpain isoforms may also be conjugated to agents affecting the targeting, stability, mobility, penetrance, bioavailability, or concentration of an inhibitor.

Selective calpain inhibitors may exist in a multitude of different forms, including nucleic acids, peptides, polypeptides, peptidomimetics, carbohydrates, lipids or other organic or inorganic molecules. Various selective inhibitors of calpain-2 according to the invention may be derived from the calpain-2 polypeptide substrate, PTEN (SEQ ID NO: 1).

Also described herein are findings that calpain-1 and calpain-2 are differentially linked—by both differential substrate specificity and differential subcellular scaffolding—to discrete cellular pathways. In particular, applicants provide evidence that administration of calpain-2 inhibitors described herein is useful for inhibiting cell death, and enhancing cognition. Indeed, calpain-1 and calpain-2 are differentially linked to the induction of Long-term Potentiation (LTP), the physiological substrate of learning and memory, in that calpain-1 is directly linked to the induction of LTP. Therefore, in aspects of the invention related to the treatment of neurological disorders, calpain-1 activation functions positively in the induction of LTP. Whereas calpain-2 activation during the same process acts like a brake in the consolidation of LTP, and thus creates a threshold for LTP, and limits the extent of LTP during the consolidation period. The particular and differential functions of calpain-1 and calpain-2 in cell protection and cell death are also disclosed herein.

In various aspects, the invention also provides methods of identifying inhibitors selective for calpain-2. These inhibitors are useful to I) inhibit cellular activity related to cell death and pathology, II) lower the threshold for sustaining LTP, III) increase LTP, IV) enhance neuronal synaptic plasticity, learning, memory, and cognition, and/or V) treat certain neurodegenerative diseases. In the methods of the invention small molecule inhibitors, proteins, peptides, polypeptides, modified peptides and polypeptides, and nucleic acids that selectively inhibit calpain-2 may be administered alone, or in combination with other inhibitors of calpain-2 function, or activators of calpain-1 function, as applicants have discovered that calpain-1 is involved specifically in neuroprotection and in the induction of synaptic plasticity, as compared to calpain-2. In another aspect, the invention relates to products and compositions such as small molecules inhibitors, polypeptides, peptides, modified peptides, and nucleic acids that selectively inhibit calpain-2 alone or in combination with other molecules that selectively inhibit calpain-2 and have diminished effect, little effect, or no measurable effect on calpain-1: In yet another aspect, the invention relates to products and compositions of matter such as small molecules inhibitors, polypeptides, peptides, modified polypeptides, and nucleic acids that selectively activate or inhibit calpain-1 function alone or in combination with other activators or selective inhibitors. Overall, the invention provides for methods of treating diseases that are susceptible to being inhibited, ameliorated, retarded, reversed, or prevented by calpain-2-selective inhibitors, or calpain-1 selective activators, or combination thereof.

### BRIEF DESCRIPTION OF THE DRAWINGS

FIG. 1: Schematic showing the respective roles of calpain-1 and calpain-2 in LTP and neurodegeneration.

FIG. 2: Field recording of excitatory postsynaptic potentials (EPSPs) performed in stratum radiatum of field CA1 in acute rat hippocampal slices in the presence or absence of the generic calpain inhibitor, Calpain inhibitor III (Z-Val-Phe-CHO). Results are expressed as percent of the average values over a 10 min baseline period and are means±S.E.M. of the indicated number of experiments. Calpain inhibitor III blocks LTP when administered before LTP induction. Compare closed circles (Calpain Inhibitor III) to closed circles (control).

FIG. 3A: Field recording of excitatory postsynaptic potentials (EPSPs) performed in stratum radiatum of field CA1 in acute rat hippocampal slices in the presence or absence of 200 nM of the calpain-2 selective inhibitor, Formula I, (mCalp-I in the drawing). Preincubation with mCalp-I enhances LTP. Results are expressed as percent of the average values over a 10 min baseline period and are means±S.E.M. of the indicated number of experiments.

FIG. 3B: Incubation of hippocampal slices with the highly selective calpain-2 inhibitor, Formula I (mCalp-I in the drawing), after Theta-burst Stimulation (TBS) results in enhanced LTP during the consolidation phase of LTP when applied from 10 min post TBS to 1 hour post TBS. Results are expressed as percent of the average values over the 10 min baseline period and are means±S.E.M. of the indicated number of experiments.

FIG. 4: Field recording of excitatory postsynaptic potentials (EPSPs) performed in stratum radiatum of field CA1 in acute hippocampal slices prepared from male UBEA mutant mice (a model of Angelman Syndrome) or their wild-type littermates in the presence or absence of 200 nM of the calpain-2 selective inhibitor, Formula I (mCalp-I in the drawing). Results are expressed as percent of the average values over a 10 min baseline period and are means±S.E.M. of the indicated number of experiments.

FIG. 5: Application of the highly selective calpain-2 inhibitor, Formula I (mCalp-I in the drawing), reduced neuronal cell death associated with extrasynaptic NMDA receptor activation in a dose-dependent fashion from 200 nM to 5 μM. Results are expressed as percent of neurons positively stained with the Hoechst reagent and are means±S.E.M. of 3-4 experiments (*p<0.01, Student's t-test).

FIG. 6: Generic calpain inhibitor, Calpain inhibitor III (Z-Val-Phe-CHO), but not the highly-selective calpain-2 inhibitor, Formula I (mCalp-I in the drawing), blocked Bic- and 4-AP-induced neuroprotection against starvation in cultured cortical neurons. Neuronal death was observed and quantified by Hoechst staining. 300-500 neurons were counted for each group in three to 6 independent experiments. *p<0.05; ns, no significant difference; one-way ANOVA followed by Bonferroni test. n=3-6. Error Bar indicates SEM

FIG. 7A: Effects of an calpain-2 selective inhibitor on fear conditioning. Formula I (mCalp-I in the drawing) was found to have a biphasic effect on learning and memory in the fear conditioning protocol. Various doses of the compound of Formula I (m-CalpI in the drawing) were injected i.p. 30 min before training to learn the association between a context or a tone with a painful stimulus. Animals were tested 24 h later for their fear responses to the context, and memory strength was quantified by the amount of time mice freeze (their biological response to fear). The ratio between the doses producing enhancement and decrease matches the ratio between the Kis to inhibit calpain-2 and calpain-1. Experiments were performed blind, as the persons analyzing the results did not know the group treatment. Results are means±S.E.M. of 8-10 experiments. *p<0.05 (ANOVA followed by Bonferroni post-test).

FIG. 7B: Effects of an calpain-2 selective inhibitor on fear conditioning. Formula 1 (mCalp-I in the drawing) was found to have a biphasic effect on learning and memory in the fear conditioning protocol. Various doses of the compound of Formula 1 (m-CalpI) were injected i.p. 30 min before training to learn the association between a context or a tone with a painful stimulus. Animals were tested 48 h later for their fear responses to the tone, and memory strength was quantified by the amount of time mice freeze (their biological response to fear). The ratio between the doses producing enhancement and decrease matches the ratio between the Kis to inhibit calpain-2 and calpain-1. Experiments were performed blind, as the persons analyzing the results did not know the group treatment. Results are means±S.E.M. of 8-10 experiments. *p<0.05 (ANOVA followed by Bonferroni post-test).

FIG. 8A: Representative images show H&E-stained ganglion cell and Plexiform layers of: i) naïve retina; ii) PBS-treated (intravitreally, 2 μI) or NMDA-treated (intravitreally, 2 μI of 2.5 mM) retina from wild-type mice that had been intraperitoneally injected with vehicle (20% DMSO), Formula I (C2l in the drawing, 0.3 mg/kg) or the pan-calpain inhibitor calpeptin (10 mg/kg) at 30 min before and 6 h after NMDA injection. H&E staining was done at 7 days after NMDA injection.

FIG. 8B: Quantitative analysis of cell number in the GCL of wild-type mice that had been injected intravitreally 7 days earlier with either 2 μI PBS or 2 μI NMDA (2.5 mM). The mice were intraperitoneally injected with vehicle (20% DMSO), a Formula I (C2l in the drawing, 0.3 mg/kg) or the pan-calpain inhibitor calpeptin (10 mg/kg) at 30 min before and 6 h after NMDA injection. Columns represent mean±S.E.M. n=4-6. *p<0.05; **p<0.01; ***p<0.001 versus NMDA plus vehicle-injected group. One-way ANOVA followed by Bonferroni test.

FIG. 8C: Quantitative analysis of thickness of the IPL of wild-type mice that had been injected intravitreally 7 days earlier with either 2 μI PBS or 2 μI NMDA (2.5 mM). The mice were intraperitoneally injected with vehicle (20% DMSO), Formula I (C2l in the drawing, 0.3 mg/kg) or the pan-calpain inhibitor calpeptin (10 mg/kg) at 30 min before and 6 h after NMDA injection.

Columns represent mean±S.E.M. n=4-6. *p<0.05; **p<0.01; ***p<0.001 versus NMDA plus vehicle-injected group. One-way ANOVA followed by Bonferroni test.

FIG. 9A: Representative immunoblot of the levels of Spectrin breakdown products (SBDP), full-length PH domain and Leucine-rich repeat Protein Phosphatase 1 (PHLPP1)α and Akt in mouse retinal extracts 6 h after intravitreal injection of PBS (control) or NMDA (2 μI of 2.5 mM). Mice were injected i.p. with vehicle (10% DMSO) or C2l (0.3 mg/kg) 30 min before intravitreal injection.

FIG. 9B: Quantitation of ratios of SBDP/Akt, as determined in retinal extracts 6 h after NMDA or PBS injections. Data represent means±S.E.M. n=4. *p<0.05, ***p<0.001,. One-way ANOVA followed by Bonferroni test.

FIG. 9C: Quantitation of ratios of PHLPP1α/Akt, as determined in retinal extracts 6 h after NMDA or PBS injections. Data represent means±S.E.M. n=4. *p<0.05, ***p<0.001,. One-way ANOVA followed by Bonferroni test.

FIG. 9D: Representative H&E staining of naive, PBS- (control) or NMDA- (2 μI of 2.5 mM) treated retina from WT mice injected i.p. with vehicle (10% DMSO) or C2l (0.3 mg/kg) 30 min before and 6 h after NMDA injection. H&E staining was performed 7 days after NMDA injection. Scale bar=30 μm.

FIG. 9E: Quantitative analysis of cell number in the GCL from wild-type mice 7 days after NMDA-injection. Six sections cut through the optic disc from each eye were analyzed. Cell numbers in GCL at a distance between 500 and 1000 μm from the optic disc were counted. Cell densities from 6 sections from each eye were averaged. Data represent means±S.E.M. n=4-8. *p<0.05, **p<0.01, One-way ANOVA followed by Bonferroni test.

FIG. 9F: Quantitative analysis of thickness of the IPL from wild-type mice 7 days after NMDA-injection. Six sections cut through the optic disc from each eye were analyzed. Cell numbers in GCL at a distance between 500 and 1000 μm from the optic disc were counted. Cell densities from 6 sections from each eye were averaged. Data represent means±S.E.M. n=4-8. *p<0.05, **p<0.01, One-way ANOVA followed by Bonferroni test.

FIG. 9G: Representative H&E staining of PBS- (control) and NMDA- (2 μI of 2.5 mM) treated retina from calpain-1 KO mice injected i.p. with vehicle (10% DMSO) or C2l (0.3 mg/kg) 30 min before and 6 h after NMDA injection. H&E stain was done 7 days after NMDA injection. Scale bar=30 μm.

FIG. 9H: Quantitative analysis of cell number in the GCL from calpain-1 KO mice 7 days after NMDA-injection. Six sections cut through the optic disc from each eye were analyzed. Cell numbers in GCL at a distance between 500 and 1000 μm from the optic disc were counted. Cell densities from 6 sections from each eye were averaged. Data represent means±S.E.M. n=6. *p<0.05, **p<0.01, ***p<0.001, One-way ANOVA followed by Bonferroni test.

FIG. 9I: Quantitative analysis of thickness of the IPL of calpain-1 mice 7 days after NMDA-injection. Six sections cut through the optic disc from each eye were analyzed. Cell numbers in GCL at a distance between 500 and 1000 μm from the optic disc were counted. Cell densities from 6 sections from each eye were averaged. Data represent means±S.E.M. n=6. *p<0.05, **p<0.01, ***p<0.001, One-way ANOVA followed by Bonferroni test.

FIG. 9J: Comparison of GCL cell numbers in NMDA-treated WT and KO mice without and with C2l treatment. n=6. **p<0.01. Two-tailed t-test.

FIG. 10A: Time course of calpain-1 and calpain-2 activation in retina after acute IOP elevation. Immunostaining of SBDP (green) in GCL and IPL of retina in WT, calpain-1 KO and C2l-injected WT mice at 0, 2, 4 and 6 h after acute IOP elevation. Sections were counterstained with DAPI (blue). C2l was injected to WT mice 2 h after IOP elevation. Scale bar=20 μm.

FIG. 10B: Time course of calpain-1 and calpain-2 activation in retina after acute IOP elevation. Immunostaining of full-length PTEN (b, red) in GCL and IPL of retina in WT, calpain-1 KO and C2l-injected WT mice at 0, 2, 4 and 6 h after acute IOP elevation. Sections were counterstained with DAPI (blue). C2l was injected to WT mice 2 h after IOP elevation. Scale bar=20 μm. (c) Quantification of SBDP staining in IPL layer, n=3-5 (eyes) at each time point. For each eye, 3 retinal sections were used for quantification. For each section, three 50×25 μm regions in the IPL layer were selected and MFI (mean fluorescence intensities) were measured and averaged. *p<0.05, **p<0.01, ***p<0.001 versus control in the same group, One-way ANOVA followed by Bonferroni test. Data represent means±S.E.M. (d) Quantification of PTEN staining, n=3-5 at each time point. *p<0.05, **p<0.01 versus control in the same group, One-way ANOVA followed by Bonferroni test.

FIG. 10C: Quantification of SBDP staining in IPL layer during time course of calpain-1 and calpain-2 activation in retina after acute IOP elevation in the retina IPL in WT, calpain-1 KO and C2l-injected WT mice at 0, 2, 4 and 6 h after acute IOP elevation. C2l was injected to WT mice 2 h after IOP elevation. n=3-5 eyes at each time point. For each eye, 3 retinal sections were used for quantification. For each section, three 50×25 μm regions in the IPL layer were selected and MFI (mean fluorescence intensities) were measured and averaged. *p<0.05, **p<0.01, ***p<0.001 versus control in the same group, One-way ANOVA followed by Bonferroni test. Data represent means±S.E.M.

FIG. 10D: Quantification of PTEN staining in IPL layer during time course of calpain-1 and calpain-2 activation in retina after acute IOP elevation in the retina IPL in WT, calpain-1 KO and C2l-injected WT mice at 0, 2, 4 and 6 h after acute IOP elevation. C2l was injected to WT mice 2 h after IOP elevation. n=3-5 at each time point. *p<0.05, **p<0.01 versus control in the same group, One-way ANOVA followed by Bonferroni test.

FIG. 11A: Calpain-2 inhibition reduces, while calpain-1 knockout exacerbates, cell death in the ganglion cell layer induced by acute IOP elevation. H&E staining of retinal sections from the right eye of vehicle- or C2l-injected wild-type and calpain-1 KO mice and the left eye where sham surgery was performed. Vehicle, 10% DMSO in PBS, was injected i.p. 30 min before and 2 h after acute IOP elevation. Pre- and post-injection C2l (0.3 mg/kg) was done i.p. 30 min before and 2 h after acute IOP elevation. For the One post injection group, C2l was injected 2 h after IOP elevation. For the Two post inj group, C2l was injected 2 and 4 h after IOP elevation. H&E staining was performed 3 days after surgery. Scale bar=30 μm.

FIG. 11B: Quantification of H&E staining shown in FIG. 11A. Six sections cut through the optic disc of each eye were analyzed. Cell numbers in GCL at a distance between 500 and 1000 from the optic disc were counted. Cell densities in 6 sections from each eye were averaged. Data represent means±S.E.M. n=7 for vehicle, n=3 for pre and post inj, n=10 for one post inj, n=6 for two post inj, n=4 for KO. ns, no significant difference. ***p<0.001, Two-tailed t-test.

FIG. 11C: Comparison of GCL cell survival rates from the right eye of vehicle- or C2l-injected wild-type and calpain-1 KO mice and the left eye where sham surgery was performed, as described in FIG. 11A. Survival rate for each mouse was calculated as the ratio of cell density in GCL of IOP-elevated eye to cell density in GCL of sham eye. *p<0.05, **p<0.01 versus vehicle, One-way ANOVA followed by Bonferroni test.

FIG. 11D: Brn-3a immunostaining in the retina of vehicle- or C2l-injected WT mice. Acute IOP elevation was performed on the right eye, while sham surgery was performed on the left eye. Vehicle, 10% DMSO, or C2l, C2l (0.3 mg/kg) was injected i.p. 2 h after acute IOP elevation. Brn3a staining was performed 3 days after surgery. Scale bar=60 μm.

FIG. 11E: Quantification of brn3a staining, as described in FIG. 11D. Brn3a-positive cells in GCL were counted. Data represents mean±S.E.M. n=4 for vehicle, n=5 for C2l. Ns, no significant difference, **p<0.01 sham versus IOP elevation, two-tailed t-test.

FIG. 11F: Comparison of survival rates. n=4 for vehicle, n=5 for C2l. **p<0.01 vehicle versus C2l, two-tailed t-test.

FIG. 11G: Representative immunoblots of PHLPP1 and STEP33 in retina tissue of WT, calpain-1 KO and C2l-injected mice collected 3 h after sham surgery or acute IOP elevation. C2l (0.3 mg/kg) was injected systemically 2 h after sham surgery or IOP elevation.

FIG. 11H: Quantitative analysis of the levels of PHLPP1 and STEP33 and ratios of pAkt/Akt for each group. Results represent means±S.E.M. of 4 experiments. *p<0.05, **p<0.01, ***p<0.001, ns no significant difference, One-way ANOVA followed by Bonferroni test.

FIG. 12A: Intravitreal injection of calpain-2 selective inhibitor reduces cell death in ganglion cell layer induced by acute IOP elevation. SBDP immunostaining in retina of calpain-1 KO mice after acute IOP elevation and intravitreal injection of vehicle or different doses of C2l. Vehicle (10% DMSO in PBS, 1 μI) or C2l (2-80 μM, 1 μI) was injected intravitreally 2 h after IOP elevation. Eyes were collected 4 h after IOP elevation for SBDP staining. Scale bar=20 μm.

FIG. 12B: Quantification of SBDP staining in IPL layer, as described in FIG. 12A. n=3 (eyes) at each concentration. In each eye, 3 retinal sections were quantified. In each section, three 50×25 μm regions in the IPL layer were selected and MFIs of SBDP signal were measured and averaged. The inhibition of SBDP signal was calculated by (MFIVehicle-MFIC2l)/MFIVehicle %. Data represents mean±S.E.M.

FIG. 12C: H&E staining in retinal sections of WT mice after IOP elevation and intravitreal injection of vehicle or C2l. Vehicle or C2l (20 μM, 1 μI) was injected 2 h after IOP elevation or sham surgery. Eyes were collected 3 days after surgery for H&E staining. Scale bar=30 μm.

FIG. 12D: Quantification of GCL cell numbers, based on H&E stains, as described in FIG. 12C. Data represent means±S.E.M, n=7 for naive, n=4 for vehicle, n=6 for C2l. *p<0.05, ***p<0.001, two-tailed t-test.

FIG. 12E: Comparison of GCL survival rates as a percentage of the control, based on H&E stains as described in FIG. 12D. *p<0.05, **p<0.01, One-way ANOVA followed by Bonferroni test.

FIG. 12F: OKR spatial frequency thresholds of eyes measured 3 days after IOP elevation or sham surgery. Vehicle or C2l (20 μM, 1 μI) was injected intravitreally 2 h after surgery. Surgery and injection were always performed in the right eye (OD). OKR of the Left eye (OS) was measured as control. Data represent means±S.E.M. n=7. ****p<0.0001 Sham plus vehicle vs. IOP elevation plus vehicle, **p<0.01 IOP elevation plus vehicle vs. IOP elevation plus C2l, One-way ANOVA followed by Bonferroni test.

FIG. 12G: OKR was re-measured 21 days after surgery, as described in FIG. 12F. n=7. ****p<0.0001, *p<0.05. One-way ANOVA followed by Bonferroni test.

FIG. 12H: RGC density in the retina of the eyes at 21 days after surgery, as described in FIG. 12F. Eyes were collected after OKR test 21 days after surgery. Brn-3a immunostaining was performed in retinal sections of the eyes. Brn-3a-positive cells in GCL were counted. Data represent means±S.E.M. n=7 for IOP elevation+C2l. n=4 for other groups. ***p<0.001, **p<0.01, One-way ANOVA followed by Bonferroni test.

FIG. 13: Retinal OCT of WT and calpain-1 KO mice after acute IOP elevation. Representative images of the eyes of animals that underwent elevated intraocular pressure. Top left panel shows an image obtained in the anesthetized mouse prior to elevated IOP (Day 0). The white arrow points to open angle with normal corneal anatomy and anterior chamber. Top right panel shows anterior chamber image 1 day after inducing IOP elevation for 1 hour; the anterior chamber synechae is visible (white arrow) along with increased hyper reflectivity in anterior chamber (red arrow) indicating breakdown of blood aqueous barrier, which is caused by proteins and cells in the anterior chamber. Also note the increased corneal thickness (yellow arrow). These features are typically seen in acute angle closure attack. Lower left panel shows image at day two; corneal thickness and anterior camber reflectivity are decreased as compared to day 1, but anterior synechae is still present. Lower right Panel is an image at day 3, showing marked decrease in cornea thickness and anterior chamber reflectivity; the synechae is now broken.

FIG. 14A: Retinal OCT of WT and calpain-1 KO mice after acute IOP elevation. Representative retinal OCT images of IOP-elevated eyes from WT and calpain-1 KO mice before (day 0) and after (day 3) IOP elevation. Scale bar=200 μm.

FIG. 14B: Quantification of the retinal thickness of IOP-elevated and sham eyes in WT mice at day 0-3 after surgery. Data represent means±S.E.M. n=6. *p<0.05 day 2 or 3 versus day 0, One-way ANOVA followed by Bonferroni test.

FIG. 14C: Quantification of the retinal thickness of IOP-elevated and sham eyes in calpain-1 KO mice at day 0-3 after surgery. Data represent means±S.E.M. n=4. *p<0.05 day 2 or 3 versus day 0, One-way ANOVA followed by Bonferroni test.

FIG. 15A: OKR test in mice. Set-up for OKR analysis in mice. Left panel, mouse head was immobilized in a home-made head restrainer, which was located in the center of a rotating grating. Right panel, saccadic pupil movements triggered by rotating gratings were recorded by an infrared camera.

FIG. 15B: Linear regression analysis of OKR spatial frequency thresholds and RGC densities measured 21 days after surgery. Black symbol, data from Sham plus Vehicle group; Blue, Sham plus C2l. Red, IOP elevation plus Vehicle. Green, IOP elevation plus C2l. N=19. R2=0.86

FIG. 16A: Treatment of cultured cortical neurons with calpain-1 C-terminal peptide results in Akt and ERK activation and neuroprotection against starvation and oxidative stress. Representative immunoblot shows that treatment of cultured cortical neurons with calpain-1 C-terminal peptide (1.5 μM) for 30 min increased Akt and ERK phosphorylation. Pre-treatment with calpain inhibitor III (10 μM) blocks the effect of calpain-1 peptide on Akt and ERK.

FIG. 16B: Quantitative analysis of the ratios of pAkt to total Akt and pERK to total ERK following: (i) treatment of cultured cortical neurons with calpain-1 C-terminal peptide (1.5 μM for 30 min.); (ii) pre-treatment with calpain inhibitor III (10 μM); or (iii) Calpain-2 C-terminal peptide (1.5 μM). Pre-treatment with Calpain-2 C-terminal peptide had no effect on Akt and ERK levels. Columns represent means±S.E.M. n=3-6. *p<0.05. One-way ANOVA followed by Bonferroni test.

FIG. 16C: Incubation of cultured cortical neurons with Calpain-1 C-terminal peptide (1.5 μM) results in neuroprotection against starvation. Columns represent means±S.E.M. n=4. *p<0.05; **p<0.01; ***p<0.001. One-way ANOVA followed by Bonferroni test.

FIG. 16D: Incubation of cultured cortical neurons with Calpain-1 C-terminal peptide (1.5 μM) results in neuroprotection against H₂O₂ insult. Columns represent means±S.E.M. n=4. *p<0.05; **p<0.01; ***p<0.001. One-way ANOVA followed by Bonferroni test.

FIG. 17: Potential calpain-2 cleavage sites in the amino acid sequence of Stargazin gamma-2 are underlined and in bold.

FIG. 18: Potential calpain-2 cleavage sites in the amino acid sequence of PTEN are underlined and in bold.

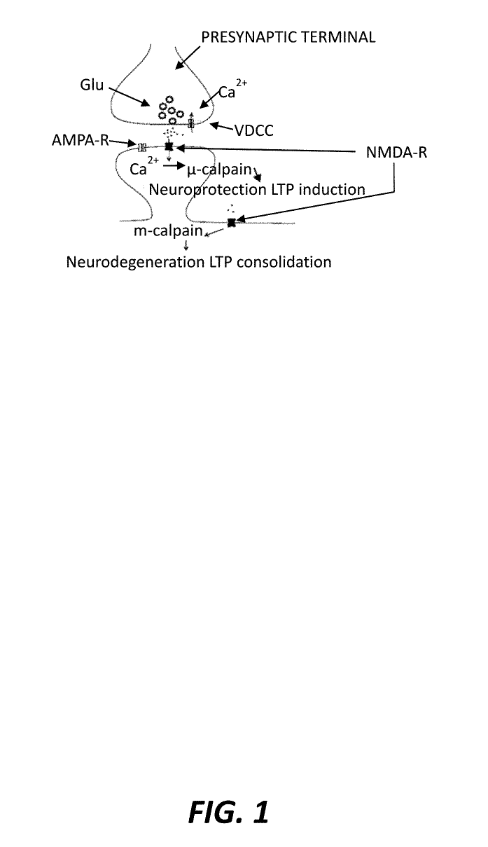

### DETAILED DESCRIPTION

As stated above, compositions and methods related to isoform-selective calpain inhibitors are described herein. In various embodiments of the invention, isoform-selective inhibitors are directed at either calpain-1, (which is also referred to by its alternative moneclature, μ-calpain) or calpain-2, (which is also referred to by its alternative moneclature, m-calpain). In particular, calpain-1 and calpain-2 refer to mammalian forms that include the calpain-1 examples of SEQ ID NOs: (2-6) and the calpain-2 examples of SEQ ID NO: (69-73) or catalytic fragment thereof. Calpains are known in the art as calcium activated neutral proteases and include a family of related molecules (Baudry et al, 2013). A catalytic fragment includes equal to or more than the first 300 amino acids of calpain-1 or calpain-2.

Generally, the term ‘inhibitor’ relates to a small molecule, peptide, polypeptide, protein, nucleic acid or modifications thereof. For example, a selective inhibitor of calpain-2, reduces its activity and has diminished, less inhibitory effect on calpain 1. On the other hand, a selective inhibitor of calpain-1 reduces its activity and has diminished inhibitory effect on calpain-2. Inhibitors of calpain-1 or calpain-2 include molecules that selectively inhibit catalytic activity competitively, non-competitively, selectively reduce their expression, selectively hasten their degradation, inhibit or hasten their chemical modification, or selectively affect protein interactions between calpain-1 or calpain-2 and one or more of its interacting proteins. Inhibitors may also comprise conjugates, compositions, or formulations that affect the targeting, stability, mobility, penetrance, bioavailability, or concentration of an inhibitor reaching the effective functional space of calpain-1 or-2.

In particular, a ‘calpain-2 selective inhibitor’ or a ‘selective calpain-2 inhibitor’ is a small molecule, peptide, polypeptide, protein, or modification thereof with a calpain-2 inhibition constant (Ki) equal to or more than 10-fold lower than its Ki for calpain-1. For instance, a Ki for calpain-2 of 0.1 μM or lower and a Ki for calpain-1 of 1 μM or higher would meet the definition of ‘calpain-2-selective inhibitor’. A ‘highly selective calpain-2 inhibitor’ would preferably exhibit at least a 20-fold lower Ki for calpain-2 than calpain-1. A calpain-1 selective inhibitor is a small molecule, peptide, polypeptide, protein, or modification thereof with a calpain-1 inhibition constant (Ki) equal to or more than 7-fold lower than its Ki for calpain-2. A highly selective calpain-1 inhibitor is a small molecule, peptide, polypeptide, protein, or modification thereof with a calpain-1 inhibition constant (Ki) equal to or more than 20-fold lower than the Ki for calpain-2. At least four highly selective calpain-2 inhibitors are disclosed in the art. Li, et al, 1996, disclose molecules 18, 19, and 56, and z-Leu-Phe-CONH-nPr is disclosed as the most highly-selective calpain-2 inhibitor with Ki for calpain-1 of 15 μM and Ki for calpain-2 as 0.05 μM in U.S. Pat. No. 6,235,929, column 14, Table I. However, the literature also describes the Ki of z-Leu-Phe-CONH-nPr for calpain-2 as being 0.35 μM and the Ki for calpain-1 as 0.05 μM (U.S. Pat. No. 6,235,929, and Li et al, 1996). Thus, the structural information disclosed in the literature for highly selective calpain-2 inhibitors is somewhat unpredictable, with one of four known highly selective calpain-2 inhibitors being of indefinite selectivity.

In various embodiments of the invention, a calpain inhibitor is a “small molecule.” A “small molecule” refers to a composition that has a molecular weight of less than 5 kD and more preferably less than about 4 kD, and most preferably less than 1 kD. Small molecules can be nucleic acids, peptides, polypeptides, peptidomimetics, carbohydrates, lipids or other organic molecules.

The terms “Polypeptide”, “Peptide”, and “Protein”, as used herein, are interchangeable and are defined to mean a biomolecule composed of amino acids linked by peptide bonds. This includes linked or individual compounds with an amine or amide bond on one end, an alpha carbon variably having two hydrogen atoms or a hydrogen atom and an R group or two R groups, a carboxylic acid group linked to the alpha-carbon on the other end, or an additional amide bond linking said amino acid with another amino acid. Polypeptides of the invention accommodate R groups of naturally occurring amino acids, including, glycine, alanine, cysteine, methionine, serine, threonine, leucine, isoleucine, valine, glutamate, aspartate, histamine, arginine, lysine, phenylalanine, tyrosine, proline, tryptophan, glutamine, and asparagine.

In various protein embodiments of the invention, a selective calpain inhibitor is an antibody that inhibits calpain-2. For example, the invention accomodates antibodies that inhibit substrate binding to calpain-2, which block access of calpain-2 to substrates, or which inhibit phosphorylation of calpain-2 at serine 50 (Shiraha et al, 2002; Zadran et al, 2010, incorporated by reference) by steric masking or allosteric modulation of calpain-2, or that bind calpain-2 such that it is inhibited. Antibodies can be polyclonal, monoclonal, single chain, anti-idiotypic, chimeric, or humanized versions of such antibodies or fragments thereof. Antibodies may be from any species in which an immune response can be raised.

Antibodies, peptides, polypeptides, and modified peptides that selectively block phosphorylation of Serine 50 of m-calpain are also contemplated, such as those described in USPPN 2003/0148264, which is incorporated by reference. These can be made using techniques defined in the art, such as phage display, a technique to generate highly variant peptide libraries as fusion proteins on the protein coat displayed on the surface of bacteriophage particles (Clackson et al, 1991; Cwirla et al, 1990, both incorporated by reference). Fusion proteins identified from a phage display library can be screened against a peptide target, such as unphosphorylated serine 50 of calpain-2. Such polypeptides can be used to selectively block phosphorylation of serine 50 by binding to, and sterically masking the phosphorylation site and thereby treat the diseases of LTP impairment disclosed herein.

‘Modifications’ refer to changes to peptides and polypeptides that are not present in peptides or polypeptides with the linked or unlinked features of any or all of naturally occuring amino acids. Modifications to the amino terminal, or carboxyl terminal, or R groups are made to increase or decrease the affinity of a peptide or polypeptide for calpain-1 or calpain-2, or increase or decrease the half-life, or increase the bioavailability, or to increase their concentration in the calpain-1 or calpain-2 target space.

Variants include polypeptides that differ in amino acid sequence due to mutagenesis. Variant proteins encompassed by the present invention are biologically active, that is they continue to possess the desired biological activity of the native protein, that is, retaining activity. In some embodiments, the variants have improved activity relative to the native protein. In further respect to the notion of ‘variants’, it is recognized that DNA sequences of a protein may be altered by various methods, and that these alterations may result in DNA sequences encoding proteins with amino acid sequences different than that encoded by a protein of the present invention.

Indeed, in various embodiments of the invention, polypeptide inhibitors may be altered in various ways including amino acid substitutions, deletions, truncations, and insertions of one or more amino acids. For example, in certain embodiments, the polypeptides of SEQ ID NOs: 1-6 and 69-73, may include up to 1, about 2, about 3, about 4, about 5, about 6, about 7, about 8, about 9, about 10, about 15, about 20, about 25, about 30, and about 35 or more amino acid substitutions, deletions, truncations, orminsertions. In yet another aspect, a sequence described herein contains 1, about 2, about 3, about 4, about 5, about 10, or about 20 additions or truncations to the C and/or N terminus region on a sequence described herein. In a non-limiting aspect, the disclosure provides for a sequence containing 1, about 2, about 3, about 4, about 5, about 10, or about 20 additions or deletions to the C and/or N terminus region of one or more of SEQ ID NOs: 1-6 and 69-73. The skilled artisan will further appreciate that changes can be introduced by mutation of the nucleotide sequences of the invention thereby leading to changes in the amino acid sequence of the encoded proteins, without altering the biological activity of the proteins. Thus, variant isolated nucleic acid molecules can be created by introducing one or more nucleotide substitutions, additions, or deletions into the corresponding nucleotide sequence disclosed herein, such that one or more amino acid substitutions, additions or deletions are introduced into the encoded protein. Mutations can be introduced by standard techniques, such as site-directed mutagenesis and PCR-mediated mutagenesis. Such variant nucleotide sequences are also encompassed by the present invention.

Conservative amino acid substitutions may be made at one or more, predicted, nonessential amino acid residues of a polypeptide calpain inhibitor according to the invention. A ‘nonessential’ amino acid residue is a residue that can be altered from the wild-type sequence of a protein without altering the biological activity, whereas an ‘essential’ amino acid residue is required for biological activity. A ‘conservative amino acid substitution’ is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

In an aspect, amino acid substitutions may be made in nonconserved regions that retain function. In general, such substitutions would not be made for conserved amino acid residues, or for amino acid residues residing within a conserved motif, where such residues are essential for protein activity. Examples of residues that are conserved and that may be essential for protein activity include, for example, residues that are identical between all proteins contained in an alignment of similar or related toxins to the sequences of the invention. Examples of residues that are conserved but that may allow conservative amino acid substitutions and still retain activity include, for example, residues that have only conservative substitutions between all proteins contained in an alignment of similar or related toxins to the sequences of the invention (e.g., residues that have only conservative substitutions between all proteins contained in the alignment homologous proteins). However, one of skill in the art would understand that functional variants may have minor conserved or nonconserved alterations in the conserved residues.

In an aspect, the disclosure provides for a protein or polypeptide having an amino acid sequence that is at least about 60%, 65%, about 70%, 75%, about 80%, 85%, about 90%, 91%, 92%, 93%, 94%, 95%, 96%, 97%, 98% or 99% identical to the aminomacid sequence of any of the sequences described herein. In another aspect, the disclosure provides for a protein or polypeptide having an amino acid sequence that is at least about 60%, 65%, about 70%, 75%, about 80%, 85%, about 90%, 91%, 92%, 93%, 94%, 95%, 96%, 97%, 98% or 99% identical to the amino acid sequence of any of the sequences described SEQ ID NOs: 1-194 and 201-22.

In another aspect, variants can include polypeptides encoded by a nucleic acid molecule that hybridizes to the nucleic acid molecules described herein, or complement thereof, under stringent conditions. Variants include polypeptides that differ in amino acid sequence due to mutagenesis. Variant proteins encompassed by the present invention are biologically active, that is they continue to possess the desired biological activity of the native protein, that is, retaining activity. In some embodiments, the variants have improved activity relative to the native protein.

It is recognized that DNA sequences of a protein may be altered by various methods, and that these alterations may result in DNA sequences encoding proteins with amino acid sequences different than that encoded by a protein of the present invention. This protein may be altered in various ways including amino acid substitutions, deletions, truncations, and insertions of one or more amino acids of SEQ ID NO: SEQ ID NO: 1-6 and 69-73, including up to 1, about 2, about 3, about 4, about 5, about 6, about 7, about 8, about 9, about 10, about 15, about 20, about 25, about 30, and about 35 or more amino acid substitutions, deletions, truncations, or insertions. In yet another aspect, a sequence described herein contains 1, about 2, about 3, about 4, about 5, about 10, or about 20 additions or truncations to the C and/or N terminus region on a sequence described herein. In a non-limiting aspect, the disclosure provides for a sequence containing 1, about 2, about 3, about 4, about 5, about 10, or about 20 additions or deletions to the C and/or N terminus region of one or more of SEQ ID NOs: 1-6 and 69-73.

### Nucleic Acid Sequence Modifications

One aspect of the invention pertains to isolated or recombinant nucleic acid molecules comprising nucleotide sequences encoding proteins or polypeptides or biologically active portions thereof, as well as nucleic acid molecules sufficient for use as hybridization probes to identify nucleic acid molecules encoding proteins with regions of sequence homology. As used herein, the term “nucleic acid molecule” is intended to include DNA molecules (e.g., recombinant DNA, cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA. An “isolated” or “recombinant” nucleic acid sequence (or DNA) is used herein to refer to a nucleic acid sequence (or DNA) that is no longer in its natural environment, for example in an in vitro or in a recombinant bacterial or plant host cell. In some embodiments, an isolated or recombinant nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For purposes of the invention, ‘isolated’ when used to refer to nucleic acid molecules excludes isolated chromosomes.

The calpain-2 substrate, Stargazin

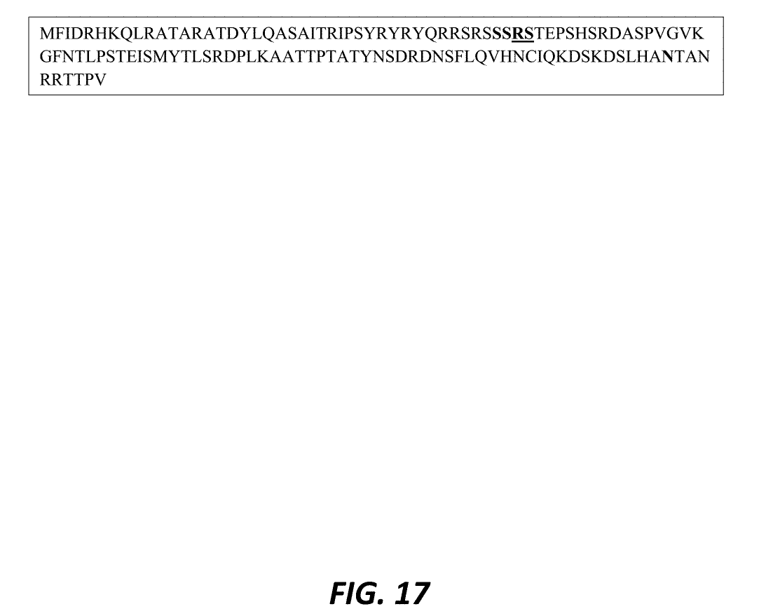

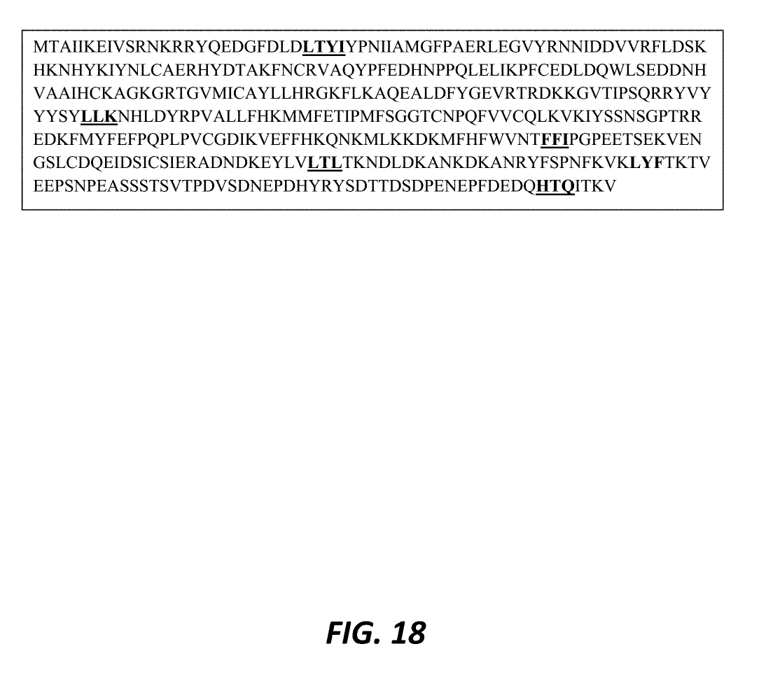

Stargazin gamma-2, a protein involved in α-amino-3-hydroxy-5-methyl-4-isoxazolepropionic acid (AMPA) receptor trafficking, is another calpain-2 specific substrate. In particular, the cytoplasmic C-terminus, shown in FIG. 17, competitively inhibits calpain-2 approximately 100-fold more than calpain-1.

The calpain-2 substrate, PTEN

The human tumor suppressor protein PTEN (SEQ ID NO: 1) is disclosed herein as having a calpain-2 specific cleavage site. Hence, PTEN is a calpain-2 specific substrate. While the literature discloses some examples of calpain-1 or calpain-2 specific substrates (i.e., Ki greater than 10 fold different), it has generally been taught that there are no differences in calpain-1 and calpain-2 substrates, in spite of the fact that calpains require, or ‘read’, a large area of their substrates and do not cleave small peptides lacking structural information (Goll et al, 2003, incorporated by reference). PTEN is the first calpain substrate with sites of significant differential sensitivity to calpain-2 versus calpain-1. Potential PTEN calpain-2 cleavage sites are underlined and in bold in FIG. 17.

Until this disclosure, target protein/substrate specificity between calpain-1 versus calpain-2 was generally unrecognized. Crystal structures of calpain-1 (Pal et al, 2003, incorporated by, reference) and calpain-2 (Horfield et al, 1999, incorporated by reference) have provided for design of calpain inhibitors, but disclosure of calpain-1 or calpain-2 selective inhibitors using said crystal structures cocrystallized with inhibitors has not taught selectivity (Cuerrier et al, 2006, incorporated by reference). Polypeptide cleavage sites sensitive to calpain-2 but insensitive to calpain-1 both in vivo and in vitro are disclosed herein. In other embodiments PTEN, or polypeptide fragments of PTEN, or modified polypeptide fragments of PTEN are calpain-2 selective inhibitors. Polypeptide fragments of PTEN, as recited in SEQ ID NO: 1, or SEQ ID NOs: 146-194, alternatively are embodiments of the invention.

Preferred embodiments of the polypeptide invention comprise polypeptides with at least about 80%, about 90%, about 95%, about 98%, about 99%, or 100% sequence identity to the peptides of SEQ ID NO: SEQ ID NO: 1, 146-194. The peptides can comprise additional modifications or domains such as those that increase targeting across the BBB, or increase the half-life in vivo or in vitro, or increase the bioavailability, or combination of modifications or other polypeptide domains thereof; for example, a transferrin polypeptide fragment, an insulin fragment, an LDL binding protein fragment, a rabies virus glycoprotein fragment. It is expected that peptides or polypeptide fragments, or modified polypeptide fragments of the calpain-2 selective cleavage sites of PTEN will inhibit calpain-2, as measured by Ki, more than calpain-1, as measured by Ki.

In various other embodiments, a polypeptide calpain inhibitor contains at least 350 consecutive amino acids of calpain-1 or calpain-2 wherein the fragment is less than the full-length native forms and has proteolytic activity. In various other embodiments, a polypeptide calpain inhibitor contains at least four consecutive amino acids of calpain-1 or calpain-2.

In various embodiments of polypeptide inhibitors of the invention, the polypeptides are substituted with groups R₁ and R₂, in which R₁ and R₂ are linked by a covalent bond. In various other embodiments, R₁ is a polypeptide fragment or modified polypeptide fragment that is a selective inhibitor of calpain-1 or calpain-2, or highly selective inhibitor of calpain-1 or calpain-2, or a molecule that improves absorption, bioavailability, half-life, or targeting such as (a transferrin polypeptide fragment, an insulin fragment, an LDL binding protein fragment, a rabies virus glycoprotein fragment); and R₂ is a polypeptide or modified polypeptide that is a selective inhibitor of calpain-1 or calpain-2, or highly selective inhibitor of calpain-1 or calpain-2, or a polypeptide fragment or modified polypeptide fragment that improves absorption, bioavailability, half-life, or targeting, such as (a transferrin polypeptide fragment, an insulin fragment, an LDL binding protein fragment, a rabies virus glycoprotein fragment).

In various embodiments, a selective inhibitor of calpain-2 according to the invention is a molecule based on the following formula:

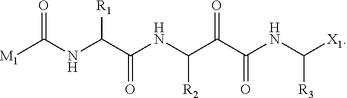

wherein: M₁ is —O, —N, —S, or —C substituted to covalently link a blocking group selected from Y₁—PhCH₂—, Y₁—Ph(CH₂)₂—, PhCH₂—Y₁, or Ph(CH₂)₂—Y₁—, wherein Y₁ is a polypeptide, or modified polypeptide covalently linked to a molecule that improves absorption, bioavailability, half-life, or targeting such as (a transferrin polypeptide fragment, an insulin fragment, an LDL binding protein fragment, a rabies virus glycoprotein fragment); or wherein Y₁ is —H, a substitution for linking small molecule, polypeptide, or modified polypeptide moieties for improving half-life, bioavailability or targeting, such as (a transferrin polypeptide fragment, an insulin fragment, an LDL binding protein fragment, a rabies virus glycoprotein fragment); or Y₁ is an —O, —N, —S, or —C substitution to link polypeptides that improve membrane permeability or blood brain barrier passage selected from (SEQ ID NOs: 195-200), a molecule that improves absorption, bioavailability, half-life, or targeting such as (a transferrin polypeptide fragment, an insulin fragment, an LDL binding protein fragment, a rabies virus glycoprotein fragment), or M₁ is —O, —N, —S, or —C substituted to covalently link a small molecule, polypeptide, or modified polypeptide that improves membrane permeability or blood brain barrier passage, a polypeptide selected from SEQ ID NOs: (195-200), a molecule that improves absorption, bioavailability, half-life, or targeting such as (a transferrin polypeptide fragment, an insulin fragment, an LDL binding protein fragment, a rabies virus glycoprotein fragment); R₁ is a functional group covalently bonded to the alpha-carbon having an L orientation, and having an amino acid side chain of leucine, phenylalanine, tyrosine, valine, isoleucine, methionine, alanine, or a modified amino acid side chain; and R₂ is —CH₃, —CH₂CH₃, —(CH₂)₂CH₃, —CH(CH₃)₂, —CH₂CH(CH₃)₂, —CH(CH₃)CH₂CH₃, —C₆H₄(4-OH), C₆H₄(3-OH), C₆H₄(2-OH), C₆H₄(2-CH₃), C₆H₄(3-CH₃), C₆H₄(4-CH₃), C₆H₄(2-OCH₃), C₆H₄(3-OCH₃), C₆H₄(4-OCH₃), C₆H₄(2-NH₂), C₆H₄(3-NH₂), C₆H₄(4-NH₂), C₆H₄(2-NHCH₃), C₆H₄(3-NHCH₃), C₆H₄(4-NHCH₃), C₆H₄(2-N(CH₃)₂), C₆H₄(3-N(CH₃)₂), or C₆H₄(4-N(CH₃)₂); R₃ is —H, —OCH₃, ═NH, —NH₂, —SH, ═O, ═S, —OCH₂CH₃, —O(CH₂)₂CH₃, —OCH(CH₃)₂, —SCH₃, —SCH₂CH₃, —S(CH₂)₂CH₃, —SCH(CH₃)₂, —OH, —CH₃, —F, —Cl, —Br, —I; X1 is —C₆H₃(3,5-R₄,R₅), —CHR₆—C₆H₃-(3,5-R₄,R₅), -2-pyridyl, -2-pyridyl(3,5, R₄,R₅), —CHR₆-2- pyridyl(3,5, R₄,R₅), -3-pyridyl(3,5, R₄, R₅), —CHR₆-3-pyridyl(3,5,R₄,R₅), -4-pyridyl(3,5, R₄, R₅), or —CHR₆-4-pyridyl(3,5,R₄,R₅); wherein R₄ is —H, —OCH₃, —OCH₂CH₃, —O(CH₂)₂CH₃, —OCH(CH₃)₂, —SCH₃, SCH₂CH₃, S(CH₂)₂CH₃, —SCH(CH₃)₂, —OH, —CH₃, —CH₂CH₃, —CN, —CHNH, —NH₂, —NHCH₃, —N(CH₃)₂, —F, —Cl, —Br, or —I; R₅ is —H, —OCH₃, —OCH₂CH₃, —O(CH₂)₂CH₃, —OCH(CH₃)₂, —SCH₃, SCH₂CH₃, —S(CH₂)₂CH₃, —SCH(CH₃)₂, —OH, —CH₃, —CH₂CH₃, —CN, —CHNH, —NH₂, —NHCH₃, —N(CH₃)₂, —F, —Cl, —Br, or —I; and R₆ is —H, —OCH₃, —OCH₂CH₃, —O(CH₂)₂CH₃, —OCH(CH₃)₂, —SCH₃, —SCH₂CH₃, —S(CH₂)₂CH₃, —SCH(CH₃)₂, —OH, —CH₃, —CH₂CH₃, —CN, —CHNH, —NH₂, —NHCH₃, —N(CH₃)₂, —F, —Cl, —Br, or —I.

In various other embodiments, a selective of calpain-2 according to the invention is a molecule based on the following formula:

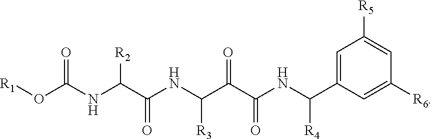

wherein: R₁ is X₁—PhCH₂—, or X₁—Ph(CH₂)₂—; wherein X₁ is —H, or a substitution for linking a molecule that improves absorption, bioavailability, half-life, or targeting such as (a transferrin polypeptide fragment, an insulin fragment, an LDL binding protein fragment, a rabies virus glycoprotein fragment); R₂ is a functional group covalently bonded to the alpha-carbon, having an L orientation, and having an amino acid side chain of leucine, phenylalanine, tyrosine, valine, isoleucine, methionine, alanine, or a modified amino acid side chain; R₃ is —CH₃, —CH₂CH₃, —(CH₂)₂CH₃, —CH(CH₃)₂—CH₂CH(CH₃)₂, —CH(CH₃)CH₂CH₃, —C₆H₅, —C₆H₄(4-OH), C₆H₄(3-OH), C₆H₄(2-OH), C₆H₄(2-CH₃), C₆H₄(3-CH₃), C₆H₄(4-CH₃), C₆H₄(2-OCH₃), C₆H₄(3-OCH₃), C₆H₄(4-OCH₃), C₆H₄(2-NH₂), C₆H₄(3-NH₂), C₆H₄(4-NH₂), C₆H₄(2-NHCH₃), C₆H₄(3-NHCH₃), C₆H₄(4-NHCH₃), C₆H₄(2-N(CH₃)₂), C₆H₄(3-N(CH₃)₂), or C₆H₄(4-N(CH₃)₂); R₄ is —H, or —OCH3, ═NH, —NH₂, —SH, ═O, ═S, —OCH₂CH₃, —O(CH₂)₂CH₃, —OCH(CH₃)₂, —SCH₃, SCH₂CH₃, —S(CH₂)₂CH₃, —SCH(CH₃)₂, —OH, —CH₃, —CH₂CH₃, —F, —Cl, —Br, or —I; R₅ is —H, —OCH₃, —OCH₂CH₃, —O(CH₂)₂CH₃, —OCH(CH₃)₂, —SCH₃, SCH₂CH₃, —S(CH₂)₂CH₃, —SCH(CH₃)₂, —OH, —CH₃, —CH₂CH₃, —CN, —CHNH, —NH₂, —NHCH₃, —N(CH₃)₂, —F, —Cl, —Br, —I; and R₆ is —H, —OCH₃, —OCH₂CH₃, —O(CH₂)₂CH₃, —OCH(CH₃)₂, —SCH₃, SCH₂CH₃, —S(CH₂)₂CH₃, —SCH(CH₃)₂, —OH, —CH₃, —CH₂CH₃, —CN, —CHNH, —NH₂, —NHCH₃, —N(CH₃)₂, —F, —Cl, —Br, or —I.

In certain embodiments, a selective inhibitor of calpain-2 according to the invention is a molecule based on the following structure of Formula I:

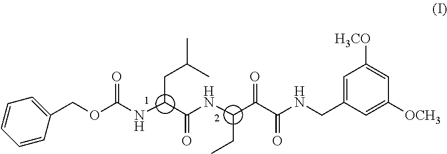 [SMILES: CCC(NC(=O)C(CC(C)C)NC(=O)OCc1ccccc1)C(=O)C(=O)NCc1cc(OC)cc(OC)c1]

wherein the chiral center 1 indicated by the circle is Levorotary (L), and wherein the chiral center 2 is D- or L-, or a racemic mixture. Two embodiments of the molecule of Formula 1 are purified forms having an L- at chiral center 1 and an L-form at chiral center 2, or separately an L-form at chiral center 1 and a D-form at chiral center 2.

In certain embodiments, a selective inhibitor of calpain-2 according to the invention is a molecule based on the following structure of Formula II:

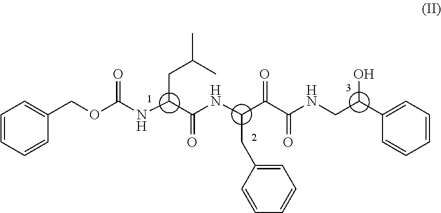 [SMILES: CC(C)CC(NC(=O)OCc1ccccc1)C(=O)NC(Cc1ccccc1)C(=O)C(=O)NCC(O)c1ccccc1]

wherein the chiral center 1 indicated by the circle is Levorotary (L), and wherein the chiral center 2 is D- or L-, or a racemic mixture, and wherein chiral center 3 is D- or L-, or a racemic mixture. Four preferred embodiments of the molecule of are purified forms having an L- at chiral center 1, an L-form at chiral center 2, and an L-form at chiral center 3; or separately an L-form at chiral center 1 and a D-form at chiral center 2, and an L-form at chiral center 3; or separately an L-form at chiral center 1 and a D-form at chiral center 2, and an D-form at chiral center 3; or separately an L-form at chiral center 1 and a L-form at chiral center 2, and an D-form at chiral center 3.

As stated above, the invention accomodates compounds having the ability to selectively inhibit calpain-2 with a Ki of at least 10-fold lower for calpain-2 than for calpain-1. For example, in various embodiments, a compound capable of inhibiting calpain-2 with a Ki of at least 10-fold lower for calpain-2 than for calpain-1 has a structure having the following structure of Formula III:

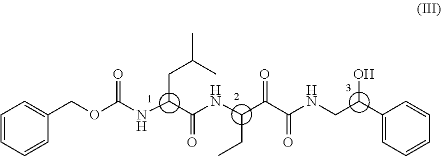 [SMILES: CCC(NC(=O)C(CC(C)C)NC(=O)OCc1ccccc1)C(=O)C(=O)NCC(O)c1ccccc1]

wherein the chiral center 1 indicated by the circle is Levorotary (L), and wherein the chiral center 2 is D- or L-, or a racemic mixture, and wherein chiral center 3 is D- or L-, or a racemic mixture. Four preferred embodiments of the molecule above are purified forms having an L- at chiral center 1, an L-form at chiral center 2, and an L-form at chiral center 3; or separately an L-form at chiral center 1 and a D-form at chiral center 2, and an L-form at chiral center 3; or separately an L-form at chiral center 1 and a D-form at chiral center 2, and an D-form at chiral center 3; or separately an L-form at chiral center 1 and a L-form at chiral center 2, and an D-form at chiral center 3.

In another embodiment of a compound capable of inhibiting calpain-2 with a Ki of at least 10-fold lower for calpain-2 than for calpain-1 has a structure having the following structure of Formula IV:

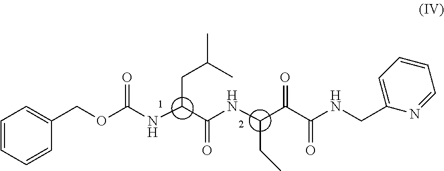 [SMILES: CCC(NC(=O)C(CC(C)C)NC(=O)OCc1ccccc1)C(=O)C(=O)NCc1ccccn1]

wherein the chiral center 1 indicated by the circle is Levorotary (L), and wherein the chiral center 2 is D- or L-, or a racemic mixture. Two preferred embodiments of the molecule of above are purified forms having an L- at chiral center 1 and an L-form at chiral center 2, or separately an L-form at chiral center 1 and D-form at chiral center 2.

In another embodiment of a compound capable of selectively inhibiting calpain-2 with a Ki of at least 10-fold lower for calpain-2 than for calpain-1 has a structure having the following structure of Formula V:

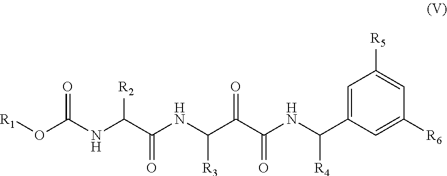

wherein R₁ is X₁—PhCH₂—, or X₁—Ph(CH₂)₂—; where X₁ is —H, or a molecule that improves absorption, bioavailability, half-life, or targeting such as (a transferrin polypeptide fragment, an insulin fragment, an LDL binding protein fragment, a rabies virus glycoprotein fragment); and wherein R₂ is a functional group covalently bonded to the alpha-carbon having an L orientation, and having an amino acid side chain of leucine, or phenylalanine, or tyrosine, or valine, or isoleucine, methionine, or alanine, or a modified amino acid side chain; and R₃ is —CH₃, or —CH₂CH₃, or —(CH₂)₂CH₃, or —CH(CH₃)₂ or —CH₂CH(CH₃)₂, or —CH(CH₃)CH₂CH₃, or —C₆H₅, —C₆H₄(4-OH), C₆H₄(3-OH), or C₆H₄(2-OH), or C₆H₄(2-CH₃), or C₆H₄(3-CH₃), C₆H₄(4-CH₃), or C₆H₄(2-OCH₃), or C₆H₄(3-OCH₃), C₆H₄(4-OCH₃), or C₆H₄(2-NH₂), or C₆H₄(3-NH₂), or C₆H₄(4-NH₂), or C₆H₄(2-NHCH₃), or C₆H₄(3-NHCH₃), or C₆H₄(4-NHCH₃), or C₆H₄(2-N(CH₃)₂), or C₆H₄(3-N(CH₃)₂), or C₆H₄(4- N(CH₃)₂); and R₄ is —H, or —OCH₃, ═NH, or —NH₂, or —SH, or ═O, or ═S, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —F, or —Cl, or —Br, or —I; and R5 is —H, or —OCH3, or —OCH2CH3, or —O(CH₂)2CH3, or —OCH(CH3)2, —SCH₃, or SCH₂CH₃, or —S(CH₂)₂CH₃, or —SCH(CH₃)₂, or —OH, or —CH₃, or —CH₂CH₃, or —CN, or —CHNH, or —NH₂, or —NHCH₃, or —N(CH₃)₂, or —F, or —Cl, or —Br, or —I; and R₆ is —H, or —OCH₃, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —CN, or —CHNH, or —NH₂, or —NHCH₃, or —N(CH₃)₂, or —F, or —Cl, or —Br, or —I.

In another embodiment of a compound capable of selectively inhibiting calpain-2 with a Ki of at least 10-fold lower for calpain-2 than for calpain-1 has a structure having the following structure of Formula VI:

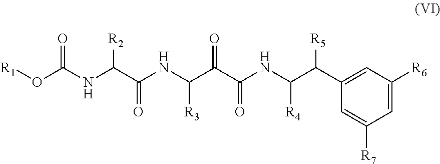

wherein R₁ is PhCH₂—, or Ph(CH₂)₂—; and wherein R₂ is a functional group covalently bonded to the alpha-carbon having an L orientation, and having an amino acid side chain of leucine, or phenylalanine, or tyrosine, or valine, or isoleucine, methionine, or alanine, or a modified amino acid side chain; and R₃ is —CH₃, or —CH₂CH₃, or —(CH₂)₂CH₃, or —CH(CH₃)₂ or —CH₂CH(CH₃)₂, or —CH(CH₃)CH₂CH₃, or —C₆H₅, —C₆H₄(4-OH), C₆H₄(3-OH), or C₆H₄(2-OH), or C₆H₄(2-CH₃), or C₆H₄(3-CH₃), C₆H₄(4-CH₃), or C₆H₄(2-OCH₃), or C₆H₄(3-OCH₃), C₆H₄(4-OCH₃), or C₆H₄(2-NH₂), or C₆H₄(3-NH₂), or C₆H₄(4-NH₂), or C₆H₄(2-NHCH₃), or C₆H₄(3-NHCH₃), or C₆H₄(4-NHCH₃), or C₆H₄(2-N(CH₃)₂), or C₆H₄(3-N(CH₃)₂), or C₆H₄(4-N(CH₃)₂); and R₄ is —H, or —OCH₃, ═NH, or —NH₂, or —SH, or ═O, or ═S, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —F, or —Cl, or —Br, or —I; and R₅ is —H, or —OCH₃, ═NH, or —NH₂, or —SH, or ═O, or ═S, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —F, or —Cl, or —Br, or —I; and R₆ is —H, or —OCH₃, or —OCH₂CH₃, or —O(CH₂)2CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —CN, or —CHNH, or —NH₂, or —NHCH₃, or —N(CH₃)₂, or —F, or —Cl, or —Br, or —I; R₇ is —H, or —OCH₃, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —CN, or —CHNH, or —NH₂, or —NHCH₃, or —N(CH₃)₂, or —F, or —Cl, or —Br, or —I, or a purine.

In another embodiment of a compound capable of selectively inhibiting calpain-2 with a Ki of at least 10-fold lower for calpain-2 than for calpain-1 has a structure having the following structure of Formula VII:

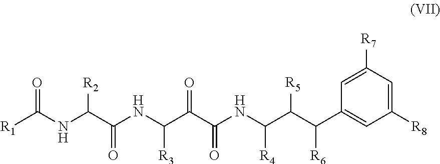

wherein R₁ is PhCH₂—, or Ph(CH₂)₂—; and wherein R₂ is a functional group covalently bonded to the alpha-carbon having an L orientation, and having an amino acid side chain of leucine, or phenylalanine, or tyrosine, or valine, or isoleucine, methionine, or alanine, or a modified amino acid side chain; and R₃ is —CH₃, or —CH₂CH₃, or —(CH₂)₂CH₃, or —CH(CH₃)₂ or —CH₂CH(CH₃)₂, or —CH(CH₃)CH₂CH₃, or —C₆H₅, —C₆H₄(4-OH), C₆H₄(3-OH), or C₆H₄(2-OH), or C₆H₄(2-CH₃), or C₆H₄(3-CH₃), C₆H₄(4-CH₃), or C₆H₄(2-OCH₃), or C₆H₄(3-OCH₃), C₆H₄(4-OCH₃), or C₆H₄(2-NH₂), or C₆H₄(3-NH₂), or C₆H₄(4-NH₂), or C₆H₄(2-NHCH₃), or C₆H₄(3-NHCH₃), or C₆H₄(4-NHCH₃), or C₆H₄(2-N(CH₃)₂), or C₆H₄(3-N(CH₃)₂), or C₆H₄(4-N(CH₃)₂); and R₄ is —H, or —OCH₃, ═NH, or —NH₂, or —SH, or ═O, or ═S, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —F, or —Cl, or —Br, or —I; and R₅ is —H, or —OCH₃, ═NH, or —NH₂, or —SH, or ═O, or ═S, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —F, or —Cl, or —Br, or —I; and R₆ is —H, or —OCH₃, ═NH, or —NH₂, or —SH, or ═O, or ═S, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —F, or —Cl, or —Br, or —I; and R₇ is —H, or —OCH₃, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —CN, or —CHNH, or —NH₂, or —NHCH₃, or —N(CH₃)₂, or —F, or —Cl, or —Br, or —I; and R₈ is —H, or —OCH₃, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —CN, or —CHNH, or —NH₂, or —NHCH₃, or —N(CH₃)₂, or —F, or —Cl, or —Br, or —I, or a purine.

In another embodiment of a compound capable of selectively inhibiting calpain-2 with a Ki of at least 10-fold lower for calpain-2 than for calpain-1 has a structure having the following structure of Formula VIII:

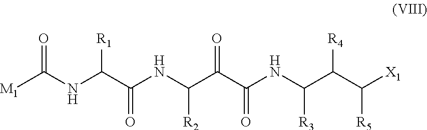

wherein M₁ is Y₁—PhCH₂—, or Y₁—Ph(CH₂)₂—, or PhCH₂—Y₁—, or Ph(CH₂)₂—Y₁—, wherein Y₁ is a covalently bound polypeptide, or modified polypeptide that improves absorption, bioavailability, half-life, or targeting such as (a transferrin polypeptide fragment, an insulin fragment, an LDL binding protein fragment, a rabies virus glycoprotein fragment); or where Y₁ is —H, or a substitution for linking small molecule, polypeptide, or modified polypeptide moieties for improving half-life, bioavailability or targeting, such as a transferrin polypeptide fragment, an insulin fragment, an LDL binding protein fragment, a rabies virus glycoprotein fragment; or Y1 is an —O, —N, —S, or —C substitution to link polypeptides that improve membrane permeability, or blood brain barrier passage, such as those of SEQ ID NO: 195-200, or a molecule that improves absorption, bioavailability, half-life, or targeting such as (a transferrin polypeptide fragment, an insulin fragment, an LDL binding protein fragment, a rabies virus glycoprotein fragment); and wherein R₂ is a functional group covalently bonded to the alpha-carbon having an L orientation, and having an amino acid side chain of leucine, or phenylalanine, or tyrosine, or valine, or isoleucine, methionine, or alanine, or a modified amino acid side chain; and R₃ is —CH₃, or —CH₂CH₃, or —(CH₂)₂CH₃, or —CH(CH₃)₂ or —CH2CH(CH₃)₂, or —CH(CH₃)CH₂CH₃, or —C₆H₅, —C₆H₄(4-OH), C₆H₄(3-OH), or C₆H₄(2-OH), or C₆H₄(2-CH₃), or C₆H₄(3-CH₃), C₆H₄(4-CH₃), or C₆H₄(2-OCH₃), or C₆H₄(3-OCH₃), C₆H₄(4-OCH₃), or C₆H₄(2-NH₂), or C₆H₄(3-NH₂), or C₆H₄(4-NH₂), or C₆H₄(2-NHCH₃), or C₆H₄(3-NHCH₃), or C₆H₄(4-NHCH₃), or C₆H₄(2-N(CH₃)₂), or C₆H₄(3-N(CH₃)₂), or C₆H₄(4-N(CH₃)₂); and R₄ is —H, or —OCH₃, ═NH, or —NH₂, or —SH, or ═O, or ═S, or —OCH₂CH₃, or —O(CH₂)₂CH₃, or —OCH(CH₃)₂, —SCH₃, or SCH₂CH₃, or —S(CH₂)2CH₃, or —SCH(CH₃)₂, or —OH, or —CH₃, or —CH₂CH₃, or —F, or —Cl, or —Br, or —I; R₅ is —H, or —OCH₃, ═NH, or —NH₂, or —SH, or ═O, or ═S, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —F, or —Cl, or —Br, or —I; wherein X₁ is —C₆H₃(3,5-R₆,R₇), or -2-pyridyl, or -2-pyridyl(3,5, R₆,R₇), or -3-pyridyl(3,5, R₆, R₇), or -4-pyridyl(3,5, R₆, R₇); wherein R₆ is —H, or —OCH₃, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —CN, or —CHNH, or —NH₂, or —NHCH3, or —N(CH₃)₂, or —F, or —Cl, or —Br, or —I; and R₇ is —H, or —OCH₃, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —CN, or —CHNH, or —NH₂, or —NHCH₃, or —N(CH₃)₂, or —F, or —Cl, or —Br, or —I.

In another embodiment of a compound capable of selectively inhibiting calpain-2 with a Ki of at least 10-fold lower for calpain-2 than for calpain-1 has a structure having the following structure of Formula IX:

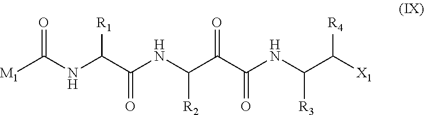

Wherein M₁ is —O, —N, —S, or —C substituted to link blocking groups such as Y₁—PhCH₂—, or Y₁—Ph(CH₂)₂—, or PhCH₂—Y₁—, or Ph(CH₂)₂—Y₁—, wherein Y₁ is a covalently linked polypeptide that improves absorption, bioavailability, half-life, or targeting such as (a transferrin polypeptide fragment, an insulin fragment, an LDL binding protein fragment, a rabies virus glycoprotein fragment); or where Y₁ is —H, or a substitution for linking a molecule that improves absorption, bioavailability, half-life, or targeting such as (a transferrin polypeptide fragment, an insulin fragment, an LDL binding protein fragment, a rabies virus glycoprotein fragment); or Y₁ is an —O, —N, —S, or —C substitution to link polypeptides that improve membrane permeability, or blood brain barrier passage, such as those of SEQ ID NO: 195-200, or a molecule that improves absorption, bioavailability, half-life, or targeting such as (a transferrin polypeptide fragment, an insulin fragment, an LDL binding protein fragment, a rabies virus glycoprotein fragment), or M₁ is —O, —N, —S, or —C substituted to covalently link other small molecules polypeptides or modified polypeptides that improve membrane permeability, or blood brain barrier passage, such as those of SEQ ID NO: 195-200, or a transferrin polypeptide fragment, or an insulin fragment, or an LDL binding protein fragment, or a rabies virus glycoprotein fragment, or a molecule that improves absorption, bioavailability, half-life, or targeting such as (a transferrin polypeptide fragment, an insulin fragment, an LDL binding protein fragment, a rabies virus glycoprotein fragment); and wherein R1 is a functional group covalently bonded to the alpha-carbon having an L orientation, and having an amino acid side chain of leucine, or phenylalanine, or tyrosine, or valine, or isoleucine, methionine, or alanine, or a modified amino acid side chain; and R₂ is —CH₃, or —CH₂CH₃, or —(CH₂)₂CH₃, or —CH(CH₃)₂ or —CH₂CH(CH₃)₂, or —CH(CH₃)CH₂CH₃, or —C₆H₄(4-OH), C₆H₄(3-OH), or C₆H₄(2-OH), or C₆H₄(2-CH₃), or C₆H₄(3-CH₃), C₆H₄(4-CH₃), or C₆H₄(2-OCH₃), or C₆H₄(3-OCH₃), C₆H₄(4-OCH₃), or C₆H₄(2-NH₂), or C₆H₄(3-NH₂), or C₆H₄(4-NH₂), or C₆H₄(2-NHCH₃), or C₆H₄(3-NHCH₃), or C₆H₄(4-NHCH₃), or C₆H₄(2-N(CH₃)₂), or C₆H₄(3-N(CH₃)₂), or C₆H₄(4-N(CH₃)₂); and R₃ is —H, or —OCH₃, ═NH, or —NH₂, or —SH, or ═O, or ═S, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —F, or —Cl, or —Br, or —I; R₄ is —H, or —OCH₃, ═NH, or —NH₂, or —SH, or ═O, or ═S, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —F, or —Cl, or —Br, or —I; wherein X₁ is —C₆H₃(3,5-R₅,R₆), or -2-pyridyl, or -2-pyridyl(3,5, R₅,R₆), or -3-pyridyl(3,5, R₅, R₆), or -4-pyridyl(3,5, R₅, R₆); wherein R₅ is —H, or —OCH₃, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —CN, or —CHNH, or —NH₂, or —NHCH₃, or —N(CH₃)₂, or —F, or —Cl, or —Br, or —I; and R₆ is —H, or —OCH₃, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —CN, or —CHNH, or —NH₂, or —NHCH₃, or —N(CH₃)₂, or —F, or —Cl, or —Br, or —I.

In another embodiment of a compound capable of selectively inhibiting calpain-2 with a Ki of at least 10-fold lower for calpain-2 than for calpain-1 has a structure having the following structure of Formula X:

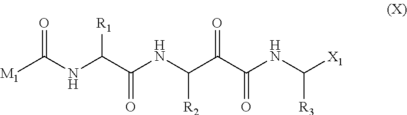

wherein M₁ is —O, —N, —S, or —C substituted to link blocking groups such as Y₁—PhCH₂—, or Y₁—Ph(CH₂)₂—, or PhCH₂—Y₁—, or Ph(CH₂)₂—Y₁—, wherein Y₁ is a polypeptide, covalently linked to a molecule that improves absorption, bioavailability, half-life, or targeting such as (a transferrin polypeptide fragment, an insulin fragment, an LDL binding protein fragment, a rabies virus glycoprotein fragment); or where Y₁ is —H, or a substitution for linking a molecule that improves absorption, bioavailability, half-life, or targeting such as (a transferrin polypeptide fragment, an insulin fragment, an LDL binding protein fragment, a rabies virus glycoprotein fragment); or Y₁ is an —O, —N, —S, or —C substitution to link polypeptides that improve membrane permeability, or blood brain barrier passage, such as those of SEQ ID NO: 195-200, or a molecule that improves absorption, bioavailability, half-life, or targeting such as (a transferrin polypeptide fragment, an insulin fragment, an LDL binding protein fragment, a rabies virus glycoprotein fragment), or M₁ is —O, —N, —S, or —C substituted to covalently link other small molecules polypeptides or modified polypeptides that improve membrane permeability, or blood brain barrier passage, such as those of SEQ ID NO: 195-200, or a molecule that improves absorption, bioavailability, half-life, or targeting such as (a transferrin polypeptide fragment, an insulin fragment, an LDL binding protein fragment, a rabies virus glycoprotein fragment); and wherein R₁ is a functional group covalently bonded to the alpha-carbon having an L orientation, and having an amino acid side chain of leucine, or phenylalanine, or tyrosine, or valine, or isoleucine, methionine, or alanine, or a modified amino acid side chain; and R₂ is —CH₃, or —CH₂CH₃, or —(CH₂)₂CH₃, or —CH(CH₃)₂ or —CH₂CH(CH₃)₂, or —CH(CH₃)CH₂CH₃, or —C₆H₅, —C₆H₄(4-OH), C₆H₄(3-OH), or C₆H₄(2-OH), or C₆H₄(2-CH₃), or C₆H₄(3-CH₃), C₆H₄(4-CH₃), or C₆H₄(2-OCH₃), or C₆H₄(3-OCH₃), C₆H₄(4-OCH₃), or C₆H₄(2-NH₂), or C₆H₄(3-NH₂), or C₆H₄(4-NH₂), or C₆H₄(2-NHCH₃), or C₆H₄(3-NHCH₃), or C₆H₄(4-NHCH₃), or C₆H₄(2-N(CH₃)₂), or C₆H₄(3-N(CH₃)₂), or C₆H₄(4-N(CH₃)₂); and R₃ is —H, or —OCH₃, ═NH, or —NH₂, or —SH, or ═O, or ═S, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —F, or —Cl, or —Br, or —I; wherein X₁ is —C₆H₃(3,5-R₄,R₅), or —CHR₆—C₆H₃-(3,5-R₄,R₅), or -2-pyridyl, or -2-pyridyl(3,5, R₄,R₅), or —CHR₆-2-pyridyl(3,5, R₄,R₅), or -3-pyridyl(3,5, R₄, R₅), —CHR₆-3-pyridyl(3,5,R₄,R₅), or -4-pyridyl(3,5, R₄, R₅), or —CHR₆-4-pyridyl(3,5,R₄,R₅); wherein R₄ is —H, or —OCH₃, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —CN, or —CHNH, or —NH₂, or —NHCH₃, or —N(CH₃)₂, or —F, or —Cl, or —Br, or —I; and R₅ is —H, or —OCH₃, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —CN, or —CHNH, or —NH₂, or —NHCH₃, or —N(CH₃)₂, or —F, or —Cl, or —Br, or —I; wherein R₆ is —H, or —OCH₃, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —CN, or —CHNH, or —NH₂, or —NHCH₃, or —N(CH₃)₂, or —F, or —Cl, or —Br, or —I.

In another embodiment of a compound capable of selectively inhibiting calpain-2 with a Ki of at least 10-fold lower for calpain-2 than for calpain-1 has a structure having the following structure of Formula XI:

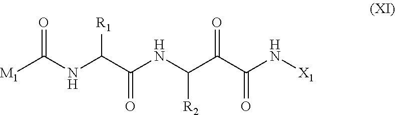

wherein M₁ is —O, —N, —S, or —C substituted to link blocking groups such as Y₁—PhCH₂—, or Y₁—Ph(CH₂)₂—, or PhCH₂—Y₁—, or Ph(CH₂)₂—Y₁—, wherein Y₁ is a polypeptide, or modified polypeptide covalently linked for improving half-life, bioavailability or targeting; or where Y₁ is —H, or a substitution for linking small molecule, polypeptide, or modified polypeptide moieties for improving half-life, bioavailability or targeting, such as (a transferrin polypeptide fragment, an insulin fragment, an LDL binding protein fragment, a rabies virus glycoprotein fragment); or Y₁ is an —O, —N, —S, or —C substitution to link polypeptides that improve membrane permeability, or blood brain barrier passage, such as those of SEQ ID NO: 195-200, or a molecule that improves absorption, bioavailability, half-life, or targeting such as (a transferrin polypeptide fragment, an insulin fragment, an LDL binding protein fragment, a rabies virus glycoprotein fragment), or M₁ is —O, —N, —S, or —C substituted to covalently link other small molecules polypeptides or modified polypeptides that improve membrane permeability, or blood brain barrier passage, such as those of SEQ ID. NO: 195-200, or a molecule that improves absorption, bioavailability, half-life, or targeting such as (a transferrin polypeptide fragment, an insulin fragment, an LDL binding protein fragment, a rabies virus glycoprotein fragment); and wherein R₁ is a functional group covalently bonded to the alpha-carbon having an L orientation, and having an amino acid side chain of leucine, or phenylalanine, or tyrosine, or valine, or isoleucine, methionine, or alanine, or a modified amino acid side chain; and R₂ is —CH₃, or —CH₂CH₃, or —(CH₂)₂CH₃, or —CH(CH₃)₂ or —CH₂CH(CH₃)₂, or —CH(CH₃)CH₂CH₃, or —C₆H₅, —C₆H₄(4-OH), C₆H₄(3-OH), or C₆H₄(2-OH), or C₆H₄(2-CH₃), or C₆H₄(3-CH₃), C₆H₄(4-CH₃), or C₆H₄(2-OCH₃), or C₆H₄(3-OCH₃), C₆H₄(4-OCH₃), or C₆H₄(2-NH₂), or C₆H₄(3-NH₂), or C₆H₄(4-NH₂), or C₆H₄(2-NHCH₃), or C₆H₄(3-NHCH₃), or C₆H₄(4-NHCH₃), or C₆H₄(2-N(CH₃)₂), or C₆H₄(3-N(CH₃)₂), or C₆H₄(4-N(CH₃)₂); wherein X1 is —C₆H₃(3,5-R₃,R₄), or -2-pyridyl, or -2-pyridyl(3,5, R₃,R₄), or -3-pyridyl(3,5, R₃, R₄), or -4-pyridyl(3,5, R₃, R₄); wherein R₃ is —H, or —OCH₃, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —CN, or —CHNH, or —NH₂, or —NHCH₃, or —N(CH₃)₂, or —F, or —Cl, or —Br, or —I; and R₄ is —H, or —OCH₃, or —OCH₂CH₃, or —O(CH₂)₂CH₃, or —OCH(CH₃)₂, —SCH₃, or SCH₂CH₃, or —S(CH₂)₂CH₃, or —SCH(CH₃)₂, or —OH, or —CH₃, or —CH₂CH₃, or —CN, or —CHNH, or —NH₂, or —NHCH₃, or —N(CH₃)₂, or —F, or —Cl, or —Br, or —I.

In another embodiment of a compound capable of inhibiting calpain-2 with a Ki of at least 10-fold lower for calpain-2 than for calpain-1 has a structure having the following structure of Formula XII:

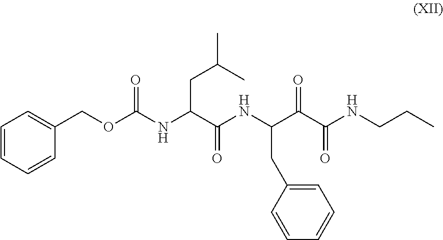 [SMILES: CCCNC(=O)C(=O)C(Cc1ccccc1)NC(=O)C(CC(C)C)NC(=O)OCc1ccccc1]

The compound in this example was characterized as having an calpain-1 Ki of 15 μM and Ki for calpain-2 of 0.05 μM in Table I, column 14 of U.S. Pat. No. 6,235,929, and the same compound is disclosed in Li et al., 1996, which features the same inventors, as having a μ-calpain Ki of 0.35 μM and m-calpain Ki of 0.05 μM (compound 53 on page 4092 of Li et al, 1996).

In another embodiment of a compound capable of inhibiting calpain-2 with a Ki of at least 10-fold lower for calpain-2 than for calpain-1 has a structure having the following structure of Formula XIII:

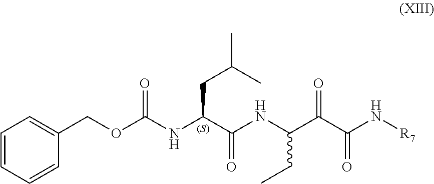

wherein R₇ is

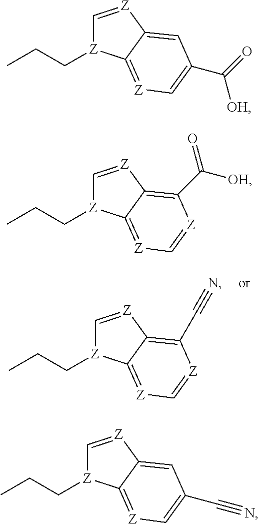

wherein Z is a carbon or a nitrogen.

In another embodiment of a compound capable of selectively inhibiting calpain-2 with a Ki of at least 10-fold lower for calpain-2 than for calpain-1 has a structure having the following structure of Formula XVI:

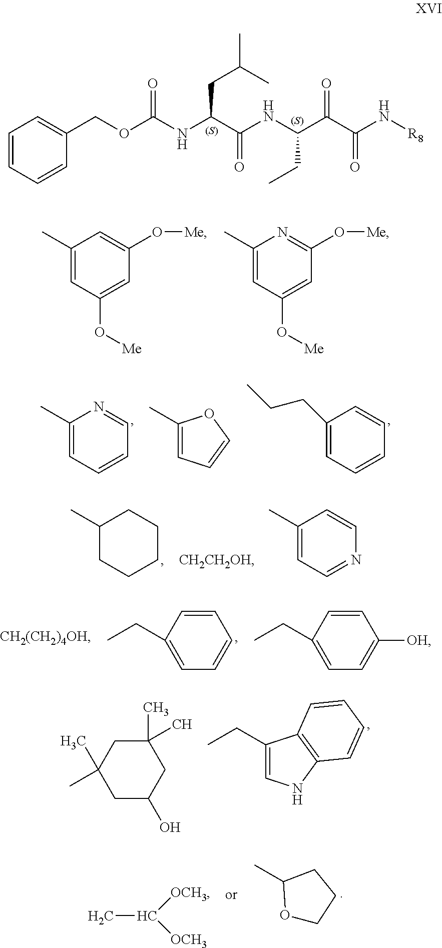

wherein R₈ is

### Methods of Identifying Isoform-Specific Calpain Substrates

Methods of identifying m-calpain specific substrates are also disclosed herein. The methods comprise contacting the substrate PTEN (SEQ ID NO: 1) or fragment or modified fragment thereof with active μ-calpain or another protein, or another prospective protein that may be cleaved by calpain-1 or calpain-2, or both, and determining if the substrate is specific for calpain-1 or calpain-2. If calpain-1 but not calpain-2 cleaves the substrate, then the substrate is a calpain-1 specific substrate. If calpain-2 but not calpain-1 cleaves the substrate, then the substrate is an calpain-2 specific substrate. The rate of substrate cleavage may also indicate the substrate is specific, or highly specific for calpain-1 or calpain-2. For example, the Kcat, or Km, or Ki can be determined in the presence of a labeled substrate prospective substrate, or in the presence of the potentially specific substrate and a second labeled non-specific substrate. Kcat, or Km, or the inhibitory constant (Ki), or another measure known in the art to define the rate of substrate cleavage can be used to indicate the rate of substrate cleavage, or the inhibitory properties of a substrate in the presence of another substrate not selective for calpain-1 or calpain-2.

In one embodiment, if the Km for a substrate is at least about 7-fold lower for calpain-1 than for calpain-2, then it is determined to be a calpain-1 specific substrate. Such substrates have the potential to serve as specific inhibitors of calpain-1. In another embodiment, if the rate of catalysis Km is at least about 20-fold lower for calpain-2 than for calpain-1, then it is determined to be a highly specific calpain-2 substrate. Such substrates have the potential to serve as highly specific inhibitors of calpain-2. In another embodiment, if the Km for the substrate is at least about 10-fold lower for calpain-1 than for calpain-2, then it is determined to be a calpain-1 specific substrate. Such substrates have the potential to serve as specific inhibitors of calpain-1. In another embodiment, if the Km is at least about 20-fold lower for calpain-1 than for calpain-2, then it is determined to be a highly specific calpain-1 substrate. Such substrates have the potential to serve as highly selective inhibitors of calpain-1.

### Methods of Identifying Isoform-Specific Calpain Cleavage Sites

In various aspects of the invention, a compound inhibitor highly selective to calpain-2, but not calpain-1, can be identified or designed based on PTEN cleavage site(s). Thus, methods of identifying a calpain-2 selective inhibitor are also disclosed herein. These methods comprise contacting a substrate, for example PTEN (SEQ ID NO: 1) or fragment or modified fragment thereof with active calpain-1 (SEQ ID NO: 2-6), or calpain-2 (SEQ ID NO: 69-73), or fragments thereof, and determining the rate of cleavage, or Kcat. Purified proteins, polypeptides, or modified polypeptides) can be contacted with a composition comprising calpain-1 or calpain-2, or purified or purified recombinant calpain-1 or calpain-2. After proteolysis, the fragments are analyzed by gel electrophoresis, collected, and subjected to Edmund degradation, or alternatively analyzed by 2-D gel and Edmund degradation, or alternatively by mass spectrometry to determine the precise cutting site of the polypeptides of the invention. Polypeptide fragments, small molecules mimicking the polypeptide fragments, or modifications of polypeptide fragments containing structure mimicking the cutting sites or peptides or polypeptides flanking the cutting sites can be used as inhibitors.

### Identifying a Calpain-2 Specific Cleavage Site.

Various methods of identifying calpain cleavage sites are known in the art. For instance, site-directed mutagenesis can be used to determine the essential elements of a calpain cleavage site (Stabach et al, 1997, incorporated herein). Isolation of cleaved fragments and subsequent Edmund degradation (Xu et al, 2007) or mass spectroscopy can be used (Chou et al, 2011). If a fragment is identified that is cleaved by calpain-2 more rapidly than by calpain-1, such fragment can be used to inhibit the cleavage of specific substrates (Xu et al, 2007). In another embodiment, such fragments can be used as an inhibitor of specific calpain isoforms.

Methods of Identifying Proteins with Calpain-1 or Calpain-2 Specificity

Recombinant PTEN can be expressed with a GST-tag in E. coli BL-21 cells in the presence of IPTG (PET15 vector) and purified with glutathione-conjugated beads or columns. In various embodiments, recombinant PTEN can be purified, isolated, and exposed to calpain-1 and calpain-2 separately and the rate of cleavage of each can be determined by measuring the appearance of cleavage products, or alternatively the cleavage rate can be measured in the presence of succinyl-Leu-Tyr-AMC or another fluorescent or difluorescent polypeptide sequence that is not selective for calpain-2 or calpain-1. The Ki of a protein suspected to exhibit calpain-1 or calpain-2 specificity can be tested by comparing the changes in the non-selective substrates exposed to calpain-2 or calpain-1.

### PDZ-Binding Domains of Calpain-1 and Calpain-2

The literature indicates that calpains play a direct role in the mediation of cell death-related signaling through extrasynaptic NMDA receptors (Li & Ju, 2012, incorporated by reference), while preliminary data indicates that synaptic activation of NMDA receptors, which induces LTP, also results in activation of the ERK pathway, and in neuroprotection. Thus, calpains are involved in competing and opposite pathways. An explanation for these dual, disparate roles is that, like other signal transduction pathways, calpainn activity is made specific through discrete scaffolding of calpains and their various substrates. Thus, for instance, even common signal transduction proteins can be differentially scaffolded to create discrete signal transducing pathways. A seminal example of this was described in the yeast high-osmolarity and mating MAPK pathways, which contain a common MAPKKK protein. Differential scaffolding of MAPKKK into each pathway mediated each independent response, with no cross-talk (Park et al, 2003, incorporated by reference). Moreover, PDZ proteins have been found to be central to separating signal transduction pathways and eliminating cross-talk (Good et al, 2011, incorporated by reference).

Scaffolding of signal transduction pathways in various cell types has been shown to be the means by which specific signals or signaling cascades are made discrete from each other. Scaffolding, or the bundling by physical association of signal transducing elements to create discrete signaling cascades that do not cross-talk has been shown to be mediated through PDZ domain-containing proteins (Good et al, 2011). It is not recognized that calpain-1 and calpain-2 have PDZ-binding domains and are scaffolded to create separate signaling cascades for calpain-1 and calpain-2. The invention also identifies calpain-1 and calpain-2 PDZ-binding domain specific peptides that displace calpain from their respective protein scaffolds: for calpain-2, TIQLDLISWLSFSVL, or fragment or modification thereof; for calpain-1 PDZ-binding domain specific peptides: VTFDLFKWLQLTMFA, or fragment or modification thereof.

As discussed above, both calpain-2 and calpain-1 have PDZ-binding domains. The PDZ-binding domains of calpain-2 versus calpain-1 are significantly different from each other, with calpain-2 being a class I PDZ-binding domain and calpain-1 domain complying with the requirements of a class II PDZ-binding domain, and thus they likely do not share PDZ domain binding partners. Since their discovery in the 1990s (Kornau et al, 1995; Woods & Bryant, 1991, all incorporated by reference), PDZ proteins have become nearly ubiquitous in eukaryotic organisms, but are much more prevalent in vertebrates. An examination of calpain-2 and calpain-1 amino acid sequences indicates that, in calpain-2 for instance, a type-I PDZ binding domain is preserved across a wide range of vertebrates. Note, for instance, that rainbow trout (Oncorhynchus mykiss), and Zebra fish (Danio rerio) present a different C-terminal sequence than the mammalian and avian vertebrates shown, but they still preserve the requirements for a type-I PDZ binding domain (S/T-X-^{ψ}; (Kang et al, 2003, incorporated by reference)). Note also the strong conservation of the type-II PDZ binding domain of calpain-1 across species (see Table 1). Thus, these sequences are strongly conserved in vertebrates, which indicates a critical functional role.

TABLE 1
C-terminal domains of calpain-1 and calpain-2 across vertebrates

| | C-terminal type Type II | C-terminal type Type I |
|---|---|---|
| | (χ-Φ- [symbol] binding | (X-S/T-X-Φ) PDZ-binding |
| Species | domain of calpain-1 | domain of calpain-2 |
| Homo sapiens | TMFA | FSVL |
| Rattus norvegicus | TMFA | FSVL |
| Ovis aries | TMFA | FSVL |
| Bos taurus | TMFA | FSVL |
| Sus scrofa | TMFA | FSVL |
| Gallus gallus | TMFA | FSVL |
| Oncorhynchus | TMFA | FTMI |
| mykiss | | |

[symbol]

indicates data missing or illegible when filed

Peptides that interfere with calpain-PDZ protein association can be easily designed and are embodiments of the invention herein. Examples of peptide inhibitors of calpain-land calpain-2 scaffolding are included herein as SEQ ID Nos: 7-68 and 74-145. and are useful in the methods of administering disclosed herein.

### Untethering Calpain-2

In various embodiments of the invention, a PDZ-binding domain of calpain-2 is used in a method of un-scaffolding calpain-2, or a phospho-mimic (replacement of serines/threonines with aspartates or glutamates) of a PDZ binding domain of calpain-2. In another embodiment, a polypeptide comprised of a calpain-2 PDZ-binding domain (SEQ ID NOs: 74-145), or peptidomimetic thereof, or a phospho-mimic of a PDZ binding domain of calpain-2 is a product that can be used in the methods of treating described herein. Peptidomimics are understood in the art as molecules that are not conventional polypeptides, but bind specifically to the same proteins of a particular polypeptide with high specificity. Scaffolding of both calpain-2 and calpain-1 help define the postsynaptic compartment space that is potentiated. A method of un-tethering calpain-2 and by administration of a calpain-2 PDZ domain is described herein. In other embodiments, a calpain-2 PDZ-binding domain is useful in a method of treating diseases of LTP impairment as described herein. A method of treating PTSD by administering a polypeptide comprised of the PDZ-binding domain of calpain-2 or peptidomimetic is an preferred embodiment of this invention. Products for the treatment of the diseases and disorders taught herein are peptides, polypeptides, modifications thereof, or peptidomimetics of the PDZ-binding domain of calpain-2. PDZ-binding domains are combined with polypeptides and modified polypeptides, or small molecule combinations or liposomes or encapsulators to enhance organ targeting, subcellular targeting, bioavailability, half-life, or potency. PDZ-binding domains can also be linked to selective inhibitors or highly selective inhibitors or formulated with selective inhibitors or highly-selective inhibitors.

### Untethering Calpain-1

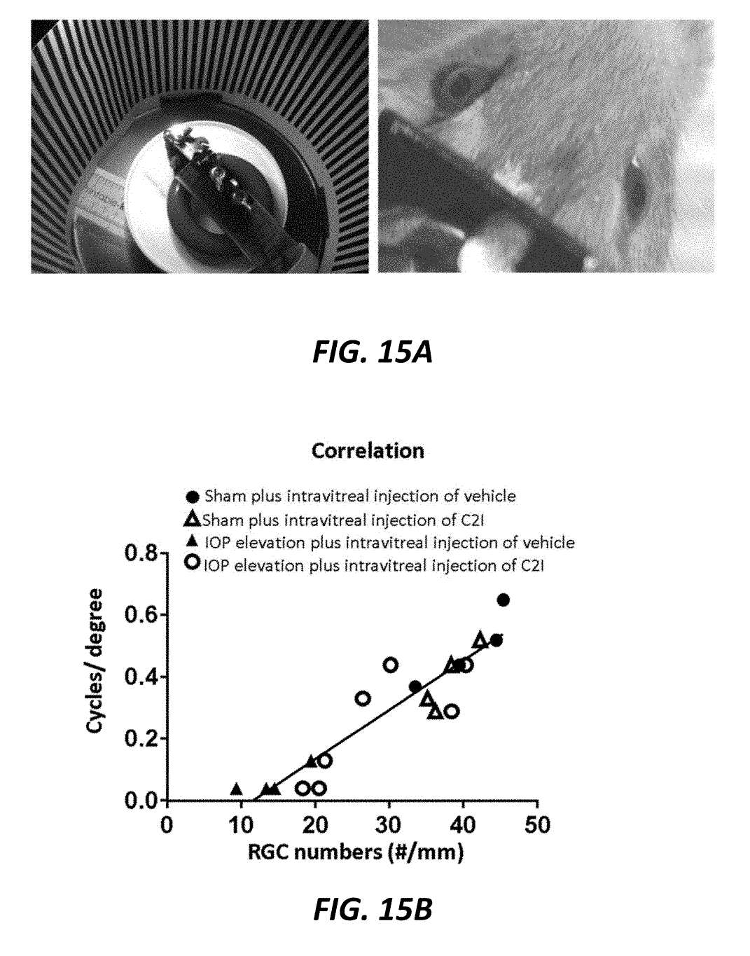

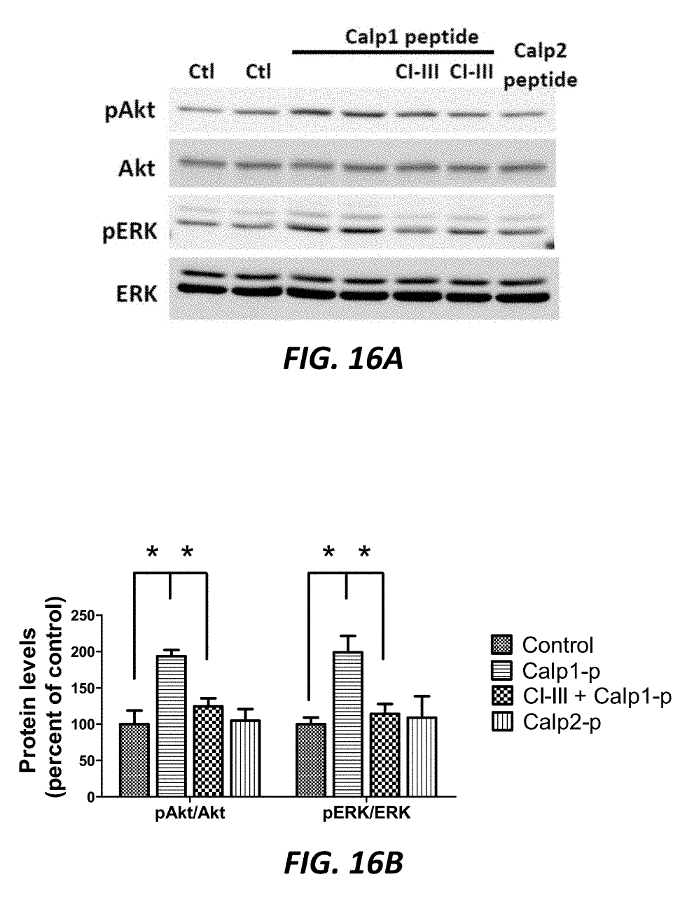

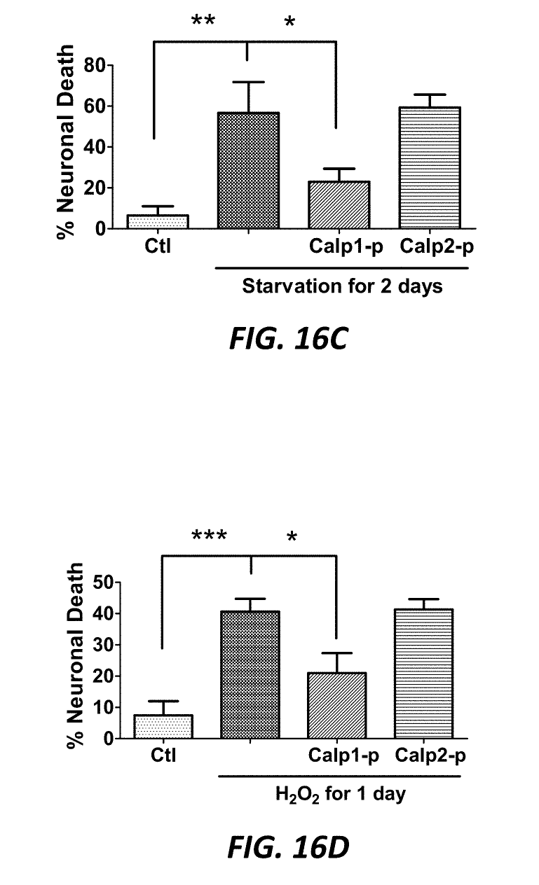

In various embodiments, the PDZ-binding domain of calpain-1 is used in a method of un-scaffolding calpain-1. In another embodiment, a polypeptide comprised of a calpain-1 PDZ-binding domain (SEQ ID NOs: 7-68) or peptidomimic thereof, or a phospho-mimic of a PDZ binding domain of calpain-1. Peptidomimics are understood in the art as molecules that are not conventional polypeptides, but bind specifically to the same proteins of a particular polypeptide with high specificity. Scaffolding of both calpain-2 and calpain-1 defines the postsynaptic compartment that is potentiated. In effect, scaffolding is participating in defining the LTP space created by activated calpain-1 versus activated calpain-2. Un-scaffolding calpain-1 with a calpain-1 PDZ-peptide results in greater LTP in rat hippocampal slices, neuroprotection by activation of the ERK/AKT pathway, and protection from serum starvation and hydrogen peroxide in culture (FIG. 15, Example 10). Products for the treatment of the diseases and disorders taught herein are peptides, polypeptides, modifications thereof, or peptidomimetics of the PDZ-binding domain of m-calpain. A calpain-1 PDZ-binding domain or peptidomimetic will be useful in the treatment of diseases characterized by impaired LTP.

### Other Polypeptide Domains

Other polypeptides comprising fusion proteins of the invention improve delivery across the blood-brain barrier, bioavailability and stability of the small molecules polypeptides, nucleic acids, modified polypeptides, or modified nucleic acids of the invention. Further embodiments of the calpain isoform-selective inhibitors are small molecule modifications and polypeptide sequences linked through a peptide linkage, or other modification. Small molecules, polypeptides, or modified polypeptides that improve passage into cells are optionally added to the inventions to improve the bioavailability of the calpain-1 selective or calpain-2 selective inhibitors of the invention. Small molecules, polypeptides, or modified polypeptides that improve delivery across the blood-brain barrier are optionally added as well. Polypeptides that can be optionally added either through a peptide bond or other modification include but are not limited to the polypeptides of SEQ ID NOs: 195-200. Approaches to maximizing delivery to the brain are optionally part of isoform-selective calpain inhibitors of the invention as well (Bertrand et al, 2010; Dufes et al, 2013; Gabathuler, 2009; Gabathuler, 2010a; Gabathuler, 2010b) and are incorporated herein by reference. Such polypeptides include polypeptide fragments from insulin, IGF-1, IGF-2, and transferrin, LDL-binding peptides, rabies virus glycoprotein.

### Liposomes, Encapsulators, Containers and Conjugates Thereof

Liposomal, nanocontainer, and encapsulating formulations with or without conjugates that improve cellular or organ targeting comprising the small molecules, polypeptides, or modified polypeptides of the invention are also embodiments of the invention. Liposomes, and conjugates for targeting across the blood brain barrier are described in the art, for example, in U.S. Pat. Nos. 6,759,058; 6,761,901; 6,849,269; 7,387,791; USPN 2011/0305751 A1; and (Schnyder & Huwyler, 2005, all of which are incorporated by reference), the contents of which are incorporated by reference herein. In another embodiment, small molecules, polypeptides and modified polypeptides of the invention are combined with carrier molecules such as liposomes or other containers or encapsulators described. In yet another embodiment, small molecules, polypeptides, nucleic acids, modified nucleic acids, or modified polypeptides of the invention are combined with pharmaceutically acceptable nanocontainers comprising a ligand for a glutathione transporter for delivery across the blood brain barrier, an insulin fragment, an insulin-like growth factor (IGF) fragment, a transferrin protein fragment, a humanized antibody to transferrin receptor, a humanized anti-E Selectin antibody a low-density lipoprotein (LDL) Receptor binding protein fragment, or a rabies glycoprotein polypeptide fragment.

### Nucleic Acid Inhibitors of Calpain-2

Nucleic acids that hybridize with the coding or untranslated 5′ or 3′ regions of calpain-2 mRNA are also embodiments of the invention. Nucleic acids are shRNA, microRNA, antisense DNA oligonucleotides or modifications thereof. Modifications that impart delivery of nucleic acids to the brain and to the locus of operational space of calpain-2 mRNA are preferred modifications, and include but are not limited to the polypeptide and liposomal conjugates disclosed herein. Methods of treating with said nucleic acids are also embodiments of the invention, and include methods of enhancing LTP, enhancing consolidation of LTP, enhancing consolidation of stimuli that normally don't induce LTP, improving memory, treating memory impairment, treating said psychiatric and neurological disease disclosed herein

### Methods of Treatment

Calpain-2 selective inhibitors are neuroprotective in cultured neurons and enhance Long-term potentiation (LTP), a cellular model of learning and memory, in acute hippocampal slices. Calpain-,2 inhibitors are useful as methods of enhancing LTP, and methods of enhancing LTP consolidation. Calpain-2 inhibitors according to the invention are useful for improving learning and reducing neurodegeneration. Therefore, they are expected to be used effectively for treatments of diseases related to synaptic dysfunction, synaptic degeneration, or neurodegeneration, including idiotypic and familial forms of Alzheimer's disease and Parkinson's disease, and dementia, Huntington's disease, Amyotropic Lateral Sclerosis (ALS), seizure, encephalitis, stroke, vasospasm, hypovolemic shock, traumatic shock, traumatic brain injury, reperfusion injury, multiple sclerosis, AIDS related dementia, neurotoxicity, head trauma, and spinal cord injury, glaucoma, open-angle glaucoma, angle-closure glaucoma, normal tension glaucoma, congenital glaucoma, pigmentary glaucoma, pseudoexfoliative glaucoma, traumatic glaucoma, neovascular glaucoma, irido corneal endothelial syndrome, ischemia in the eye, ischemia in the retina.

In various embodiments, calpain-2 inhibitors according to the invention are useful for effectively treating hearing loss, including hearing loss as a consequence of ototoxicity due to damage of the auditory nerve, for example, as a side effect of a drug or toxin. In various other embodiments, calpain-2 inhibitors according to the invention are useful for effectively treating hearing loss as a consequence of neurodgeneration.

In various embodiments, calpain-2 inhibitors according to the invention are useful for effectively treating Wolfram syndrome 1, while in other embodiments, calpain-2 inhibitors according to the invention are useful for effectively treating Wolfram syndrome 2. In certain embodiments calpain-2 inhibitors according to the invention treats neurodegeneration associated with Wolfram Syndrome 1 or 2 by inhibiting calpain-2 activity, including calpain-2 activity that is increased as a consequence of disregulation of either WSF1 or WSF2 gene expression.

In various embodiments, an effective amount of calpain-2 inhibitor according to the invention is administered to a patient in need thereof to inhibit neuronal cell death. In various other embodiments, an effective amount of calpain-2 inhibitor according to the invention is administered to a patient in need thereof to enhance memory. In yet other embodiments, an effective amount of calpain-2 inhibitor according to the invention is administered to a patient in need thereof to treat a neurological disorder. In yet another embodiment, an effective amount of calpain-2 inhibitor according to the invention is administered to a patient in need thereof to treat glaucoma.

In various other embodiments of the invention, effective amounts of a calpain-2 selective inhibitor, according to the invention, are used to effectively to treat diseases of synaptic and behavioral dysfunction, which include but are not limited to Schizophrenia, Autism Spectral Disorders, Bipolar Illness, drug-induced psychosis, Post-Traumatic Stress Disorder (PTSD), depression and suicidal thoughts, phobias, obsessive-compulsive disorder, trisomy 21, ADHD, and ADD. Autism Spectral Disorder includes Autistic disorder, (classic autism), Angelman Syndrome, Asperger's disorder (Asperger syndrome), Pervasive developmental disorder not otherwise specified (PDD-NOS), Rett's disorder (Rett syndrome), Childhood disintegrative disorder (CDD).

### Compositions

The pharmaceutical compositions of the invention may be prepared by methods known in the pharmaceutical formulation art, for example, see Remington's Pharmaceutical Sciences, 22nd Ed., (Pharmaceutical Press, 2012), which is incorporated herein by reference. In a solid dosage form, a compound of the invention may be admixed with at least one pharmaceutically acceptable excipient such as, for example, sodium citrate or dicalcium phosphate or (a) (a) fillers or extenders, such as, for example, starches, lactose, sucrose, glucose, mannitol, and silicic acid, (b) binders, such as, for example, cellulose derivatives, starch, aliginates, gelatin, polyvinylpyrrolidone, sucrose, and gum acacia, (c) humectants, such as, for example, glycerol, (d) disintegrating agents, such as, for example, agar-agar, calcium carbonate, potato or tapioca starch, alginic acid, croscarmellose sodium, complex silicates, and sodium carbonate, (e) solution retarders, such as, for example, paraffin, (f) absorption accelerators, such as, for example, quaternary ammonium compounds, (g) wetting agents, such as, for example, cetyl alcohol, and glycerol monostearate, magnesium stearate and the like (h) adsorbents, such as, for example, kaolin and bentonite, and (i) lubricants, such as, for example, talc, calcium stearate, magnesium stearate, solid polyethylene glycols, sodium lauryl sulfate, or mixtures thereof. In the case of capsules, tablets, and pills, the dosage forms may also comprise buffering agents.

Pharmaceutically acceptable adjuvants known in the pharmaceutical formulation art may also be used in the pharmaceutical compositions of the invention. These include, but are not limited to, preserving, wetting, suspending, sweetening, flavoring, perfuming, emulsifying, and dispensing agents. Prevention of the action of microorganisms may be ensured by inclusion of various antibacterial and antifungal agents, for example, parabens, chlorobutanol, phenol, sorbic acid, and the like. It may also be desirable to include isotonic agents, for example, sugars, sodium chloride, and the like. If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

Solid dosage forms as described above may be prepared with coatings and shells, such as enteric coatings and others, as is known in the pharmaceutical art. They may contain pacifying agents, and can also be of such composition that they release the active compound or compounds in a certain part of the intestinal tract in a delayed manner. Non-limiting examples of embedded compositions that may be used are polymeric substances and waxes. The active compounds may also be in microencapsulated form, if appropriate, with one or more of the above-mentioned excipients.

Suspensions, in addition to the active compounds, may contain suspending agents, such as, for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like. Liquid dosage forms may be aqueous, may contain a pharmaceutically acceptable solvent as well as traditional liquid dosage form excipients known in the art which include, but are not limited to, buffering agents, flavorants, sweetening agents, preservatives, and stabilizing agents.

In addition to the topical method of administration described above, there are various methods of administering the compounds of the invention topically to the lung. One such means could involve a dry powder inhaler formulation of respirable particles comprised of the compounds of the invention, which the patient being treated inhales. It is common for a dry powder formulation to include carrier particles, to which the compound particles can adhere to. The carrier particles may be of any acceptable pharmacologically inert material or combination of materials. For example, the carrier particles may be composed of one or more materials selected from sugar alcohols; polyols, for example sorbitol, mannitol or xylitol, and crystalline sugars, including monosaccharides and disaccharides; inorganic salts such as sodium chloride and calcium carbonate; organic salts such as sodium lactate; and other organic compounds such as urea, polysaccharides, for example cyclodextrins and dextrins. The carrier particles may be a crystalline sugar, for example, a monosaccharide such as glucose or arabinose, or a disaccharide such as maltose, saccharose, dextrose or lactose, The compound of the invention would be dispersed into the respiratory tract, and subsequently contact the lower lung in a pharmaceutically effective amount.

In addition to the topical method of administration described above, there are various methods of administering the compounds of the invention systemically by such methods. One such means would involve an aerosol suspension of respirable particles comprised of the compounds of the invention, which the patient being treated inhales. The compound would be absorbed into the bloodstream via the lungs in a pharmaceutically effective amount. The respirable particles can be liquid or solid, with a particle size sufficiently small to pass through the mouth and larynx upon inhalation.

Dosage forms for oral administration, which includes capsules, tablets, pills, powders, granules, and suspensions may be used. Dosage forms for pulmonary administration, which includes metered dose inhaler, dry powder inhaler or aerosol formulations may be used. In such solid dosage forms, the active compound may be mixed with at least one inert, pharmaceutically acceptable excipient (also known as a pharmaceutically acceptable carrier).

A compound according to the invention may also be used to formulate liquid or injectable pharmaceutical compositions. Administration of a compound of the invention in pure form or in an appropriate pharmaceutical composition may be carried out via any of the accepted modes of administration or agents for serving similar utilities. Thus, administration may be, for example, orally, buccally, nasally, pulmonary, parenterally (intravenous, intramuscular, intraperitoneal, or subcutaneous), topically, transdermally, intravaginally, intravesically, intrasystemically, ophthalmically or rectally, in the form of solid, semi-solid, lyophilized powder, or liquid dosage forms, such as, for example, tablets, suppositories, pills, soft elastic and hard gelatin capsules, powders, solutions, suspensions, or aerosols, or the like, such as, for example, in unit dosage forms suitable for simple administration of precise dosages. One route of administration may be oral administration, using a convenient daily dosage regimen that can be adjusted according to the degree of severity of the condition to be treated.

### EXAMPLES

### Example 1

A Small Molecule/Modified Polypeptide Highly Selective for Calpain-2

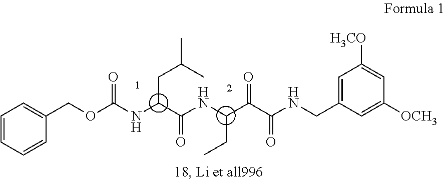 [SMILES: CCC(NC(=O)C(CC(C)C)NC(=O)OCc1ccccc1)C(=O)C(=O)NCc1cc(OC)cc(OC)c1]

Formula 1, where chiral center 1 is the L-form and chiral center 2 is a racemic mixture of D- and L- in this example was introduced at various concentrations (from 1 nM to 10 μM) into an in vitro mix comprising succinic-Leu-Tyr-AMC and calpain-1 or calpain-2 (Sasaki et al, 1984), and the kinetics of the loss of fluorescence were determined for each of the calpains (Powers et al, 2000). Ki values obtained for the compound in the literature are 2.3 μM for calpain-1 and 0.022 μM for calpain-2 (Li et al, 1996). However, the Ki of calpain-1 was re-determined herein to be 1.29 μM±0.7 μM, and the Ki for calpain-2 was determined to be 0.025 μM±0.02 μM. Therefore, the assessment of selectivity described herein was different than the prior teaching. This compound is an inhibitor highly selective for calpain-2 because its Ki is more than 50-fold lower for calpain-2 than for calpain-1.

### Example 2

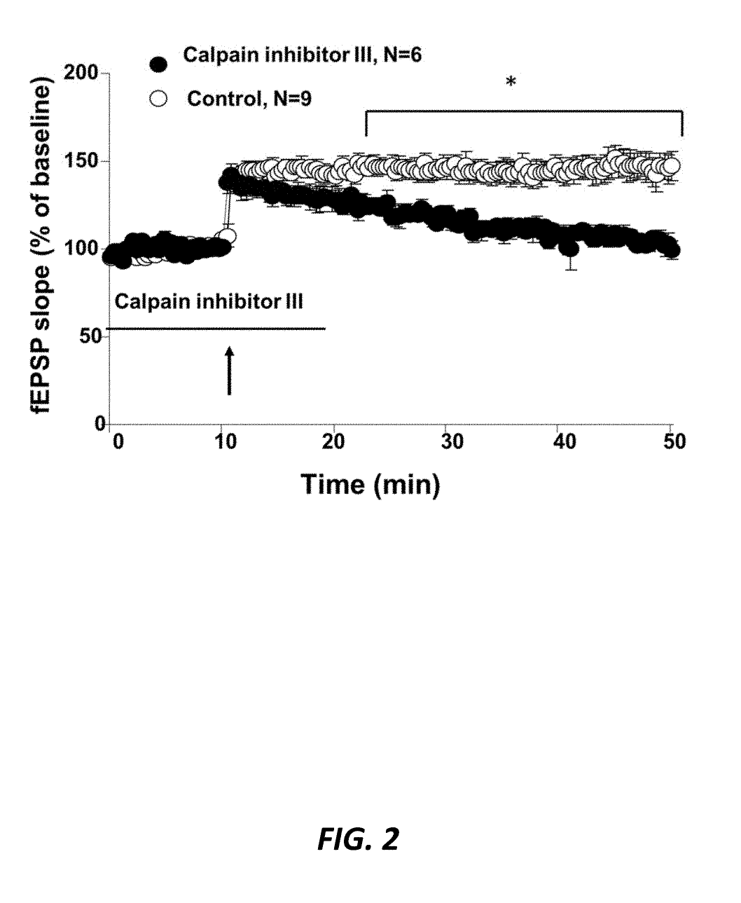

Generic calpain inhibitors block LTP when administered before LTP induction. Field recording of excitatory postsynaptic potentials (EPSPs; FIG. 2) was performed in stratum radiatum of field CA1 in acute rat hippocampal slices. Ten μM Calpain Inhibitor III (Z-Val-Phe-CHO; Ki for both calpain-1 and calpain-2: ~8 nM), which inhibits both calpain-1 and calpain-2, was added prior to Theta-burst stimulation (TBS; 10 bursts of 4 pulses at 100 Hz with 200 ms between bursts), which can be used to elicit LTP (Capocchi et al, 1992). Preincubation with the non-selective calpain inhibitor, Calpain inhibitor III, did not block short-term potentiation, the increase in fEPSPs that follows TBS, but prevented the formation of LTP, when compared to control (compare open circles to filled circles).

### Example 3

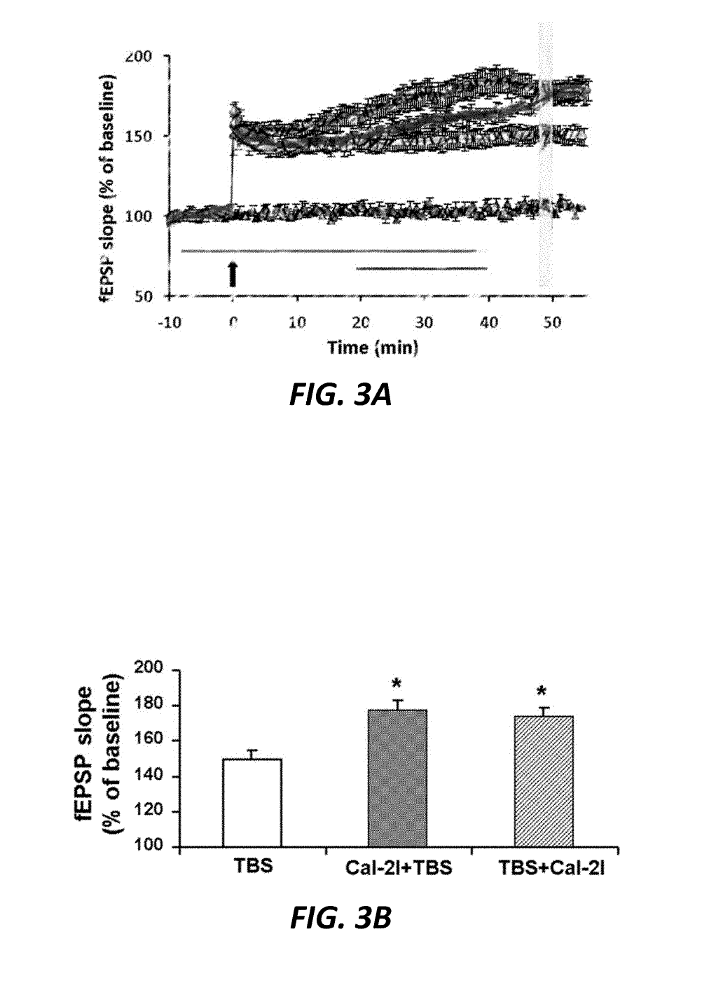

A calpain 2-selective inhibitor enhances LTP. Acute hippocampal slices were prepared and bathed in ACSF. 200 nM calpain-2-selective inhibitor of Formula 1, which inhibits calpain-2 50-100 fold more than calpain-1, was administered prior to Theta-burst stimulation, which has the ability to elicit LTP (see line #1 of FIG. 3A for administration time-course). In unexpected contrast to administration of a non-selective calpain inhibitor such as calpain inhibitor III, preincubation with the calpain-2 selective inhibitor does not inhibit LTP (FIG. 3A); it enhances it. Incubation of hippocampal slices with the same highly selective calpain-2 inhibitor after Theta-burst Stimulation (TBS) also results in enhanced LTP during the consolidation phase of LTP when applied from 10 min post TBS to 1 hour post TBS.

### Example 4

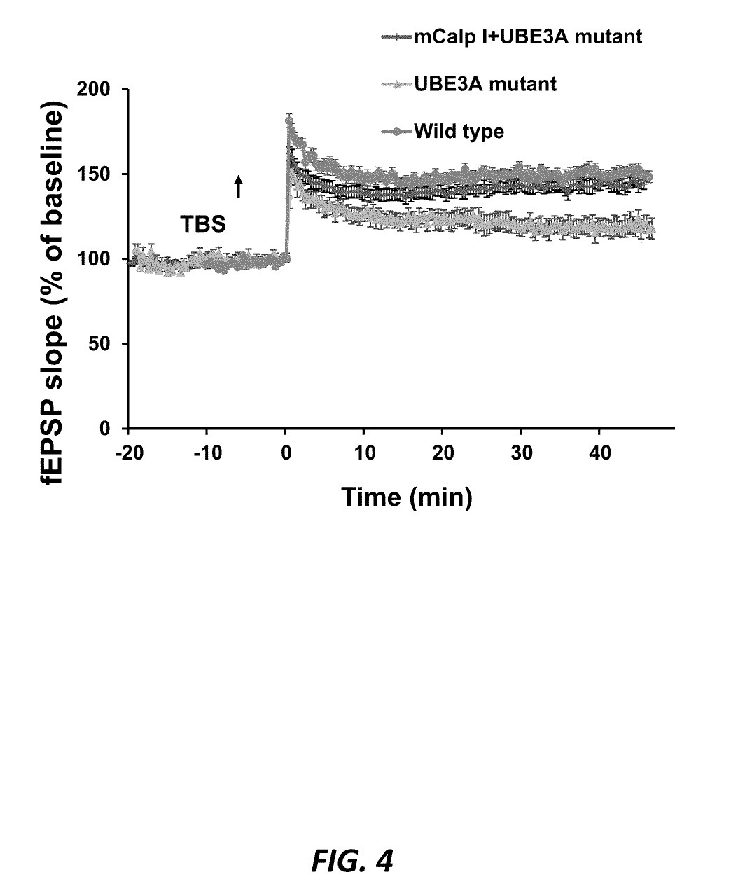

A calpain 2-specific inhibitor rescues LTP impairment in hippocampal slices from a mouse model of Angelman Syndrome. Field recording of excitatory postsynaptic potentials (EPSPs) was performed in stratum radiatum of field CA1 in acute hippocampal slices prepared from male UBEA mutant mice or their wild-type littermates. After 20 min of baseline recording, theta burst stimulation (TBS, arrow of FIG. 4 was applied to the Schaffer collateral pathway to induce LTP. A specific calpain-2 inhibitor (mCal-I; Example 1 was applied (200 nM), as indicated by the solid horizontal line (FIG. 4). While it has no effect on the initial increase in fEPSP elicited by TBS, it restored LTP magnitude to the level found in slices from wild-type mice. Results are means±S.E.M. of 6-7 slices from 3-4 animals.

### Example 5

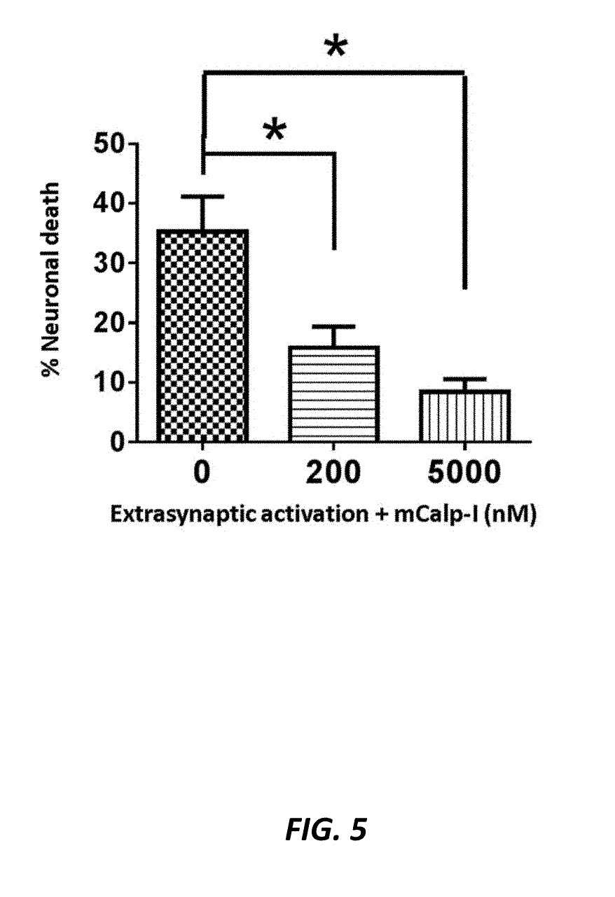

A selective calpain-2 inhibitor blocks neuronal death mediated by extrasynaptic NMDA receptor activation. Cortical neuronal cultures (14 DIV) were treated to induce selective activation of extrasynaptic NMDA receptors, which results in neuronal cell death. Application of the highly selective calpain-2 inhibitor of Formula 1 reduced the neuronal cell death associated with extrasynaptic NMDA receptor activation in a dose-dependent fashion from 200 nM to 5 μM (FIG. 5).

### Example 6

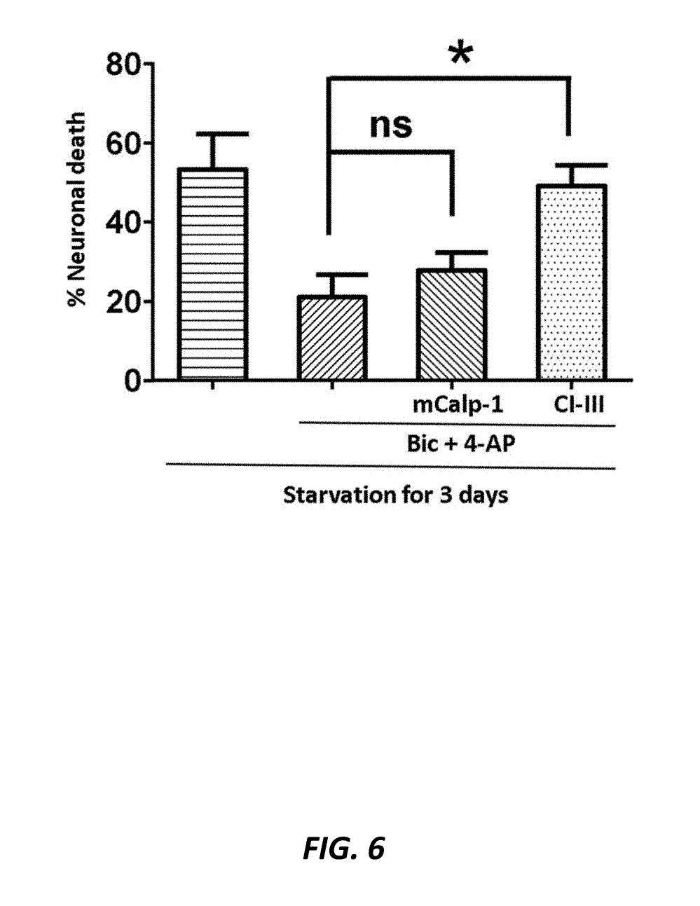

Highly-selective calpain 2 inhibitors do not interfere with synaptic activity resulting in neuroprotection. Calpain inhibitor-III (which is not a selective calpain inhibitor), but not mCalp-I (200 nM) blocked Bic- and 4-AP-induced neuroprotection against starvation in cultured cortical neurons. Neuronal death was observed and quantified by Hoechst staining. 300-500 neurons were counted for each group in three to 6 independent experiments. *p<0.05; ns, no significant difference; one-way ANOVA followed by Bonferroni test. n=3-6. Error Bar indicates SEM (FIG. 6).

### Example 7

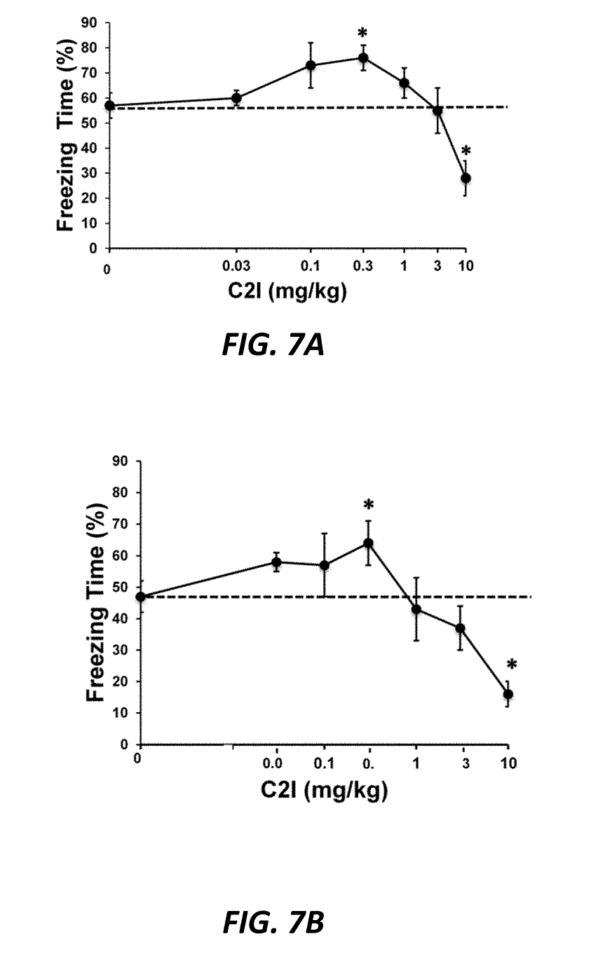

Formula 1 Enhances Memory. Formula 1 was found to have a biphasic effect on learning and memory in the fear conditioning protocol. In this protocol, mice were trained to learn the association between a context or a tone with a painful stimulus. Various doses of the compound of Formula 1 (mCalp-I) were injected i.p. 30 min before training. Animals were tested 24 h later in the context (FIG. 7A) and 48 h later with the tone (FIG. 7B). When tested 24 h or 48 h later for their fear responses to either the context or the tone, memory strength was quantified by the amount of time mice freeze (their biological response to fear). The ratio between the doses producing enhancement and decrease matches the ratio between the Kis to inhibit calpain-2 and calpain-1. Experiments were performed blind, as the persons analyzing the results did not know the group treatment. Results are means±S.E.M. of 8-10 experiments. *p<0.05 (ANOVA followed by Bonferroni post-test).

### Example 8

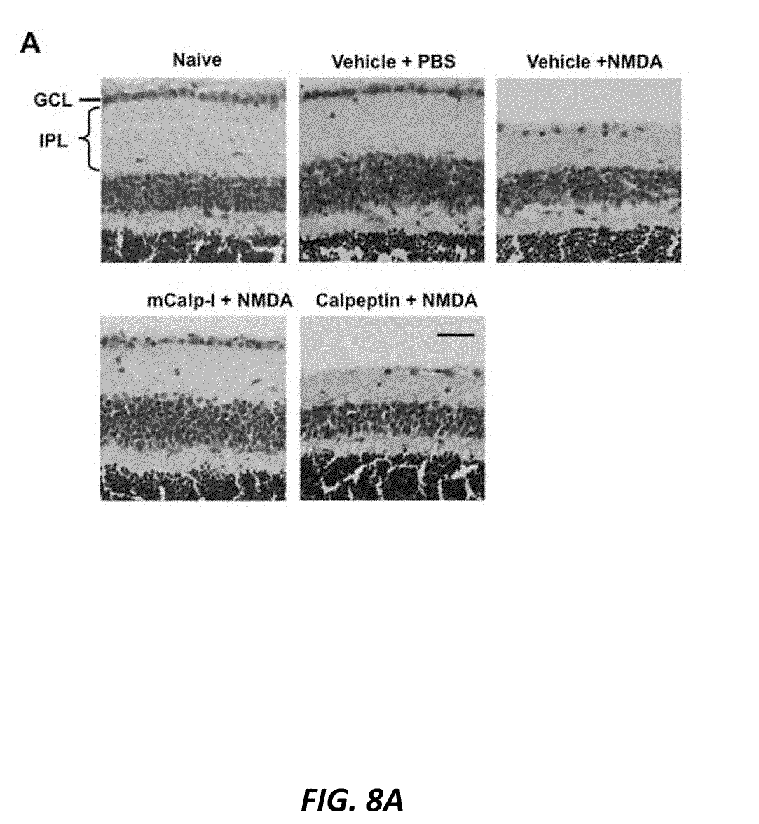

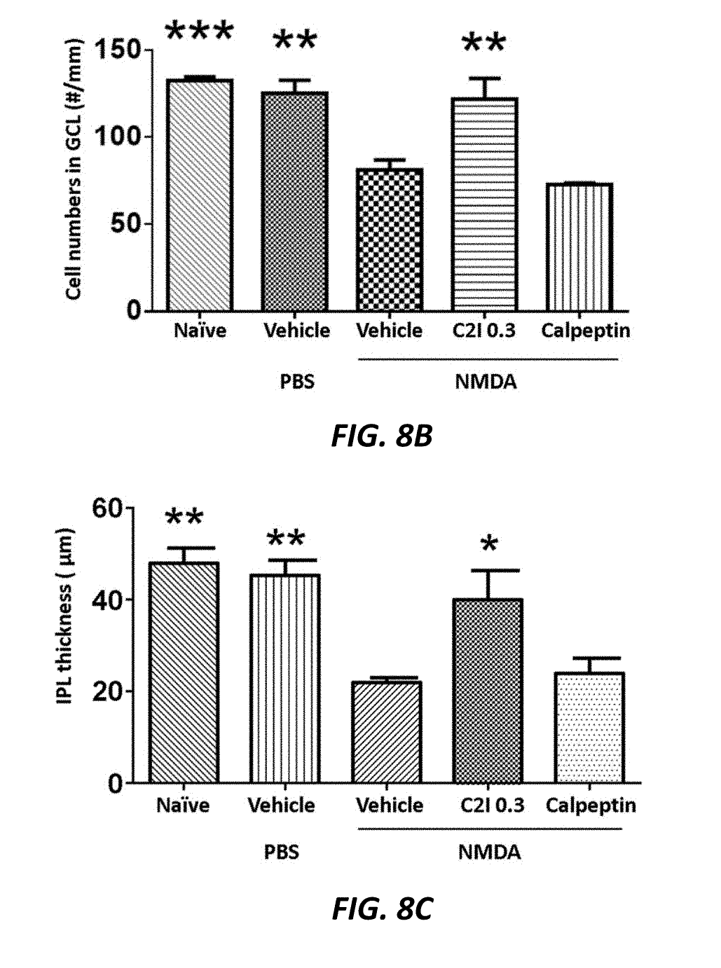

Intraperitoneal injection of calpain-2 selective inhibitor is protective against NMDA-induced retinal damage. Either 2 μI PBS or 2 μI NMDA (2.5 mM) was injected intravitreally into the retinas of wild-type mice that had been intraperitoneally injected with vehicle (20% DMSO), a calpain-2 selective inhibitor (C2l, Z-Leu-Abu-CONH—CH2-C6H3 (3, 5-(OMe)2)13,14-0.3 mg/kg) or the pan-calpain inhibitor calpeptin (10 mg/kg) at 30 min before and 6 h after NMDA injection. H&E staining was done at 7 days after NMDA injection. See FIG. 8A. Quantitative analysis of cell number in the GCL and IPL 7 days after NMDA-injection were also performed. See FIGS. 7B and 7C, respectively.

### Example 9

Calpain-1 and calpain-2 play opposite roles in retinal damage induced by intravitreal NMDA injection. Calpain activity is involved in retinal cell death induced by NMDA Receptor (R) activation. To test the specific roles of calpain-1 and calpain-2 in this process, wild-type (WT) mice were injected systemically with a calpain-2 selective inhibitor (C2l), Z-Leu-Abu-CONH—CH2-C₆H3 (3, 5-(OMe)2)13,14, 30 min before NMDA intravitreal injection, as described in Example 8. Levels of spectrin breakdown products (SBDP), the cleaved products of spectrin by both calpain-1 and -2, and of PH domain and Leucine-rich repeat Protein Phosphatase 1 (PHLPP1), a protein degraded by calpain-1 following NMDAR activation, were determined in retinal extracts 6 h after NMDA injection (FIG. 8(A-C)). Akt levels were also measured as a loading control. Levels of SBDP were significantly increased and those of PHLPP1 decreased after NMDA injection, as compared to control (PBS intravitreal injection), suggesting that calpain was activated after NMDA injection. Systemic (intraperitoneal; i.p.) injection of C2l significantly suppressed NMDA-induced changes in SBDP but not in PHLPP1, suggesting that C2l systemic injection selectively inhibited calpain-2 but not calpain-1 activation in retina after intravitreal NMDA injection.

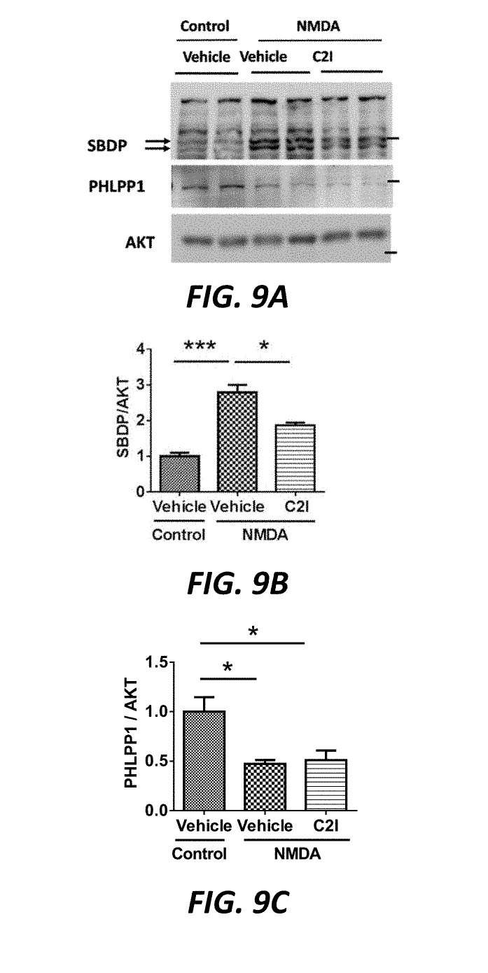

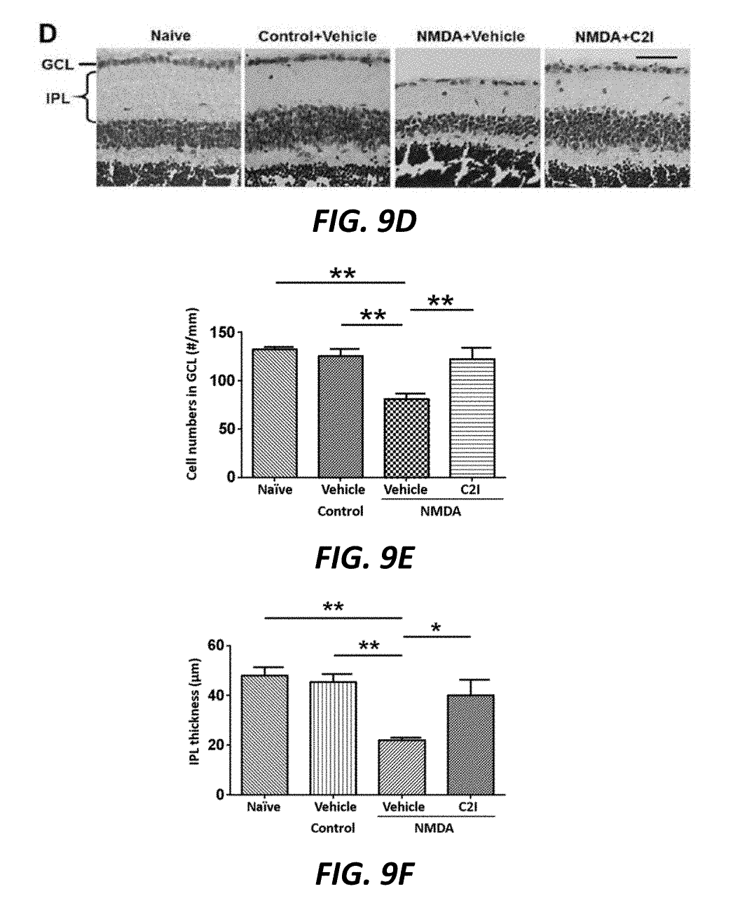

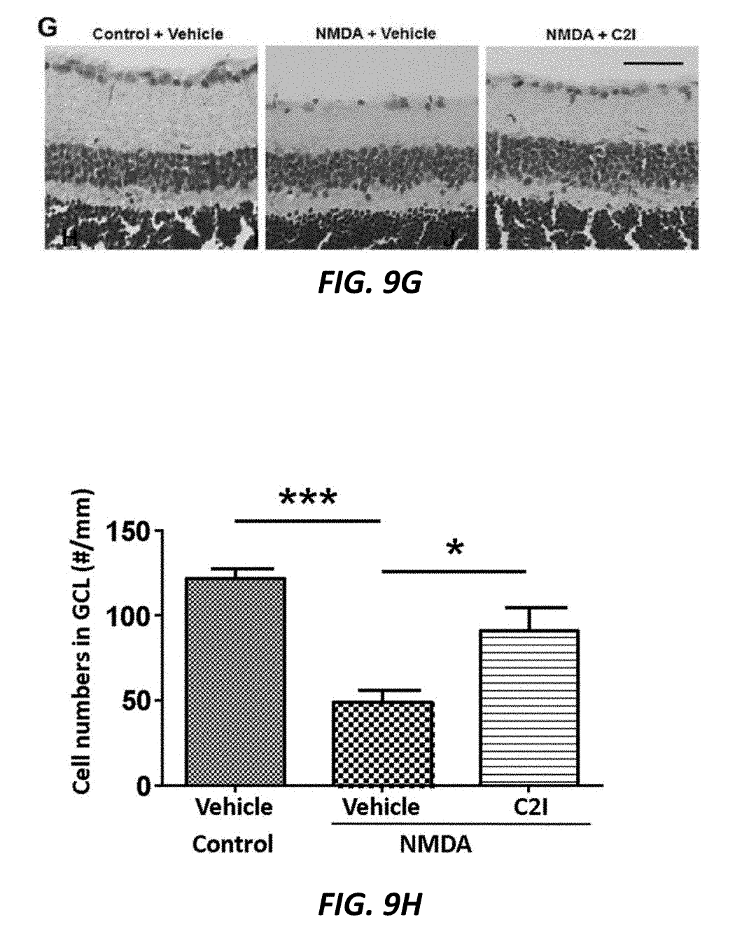

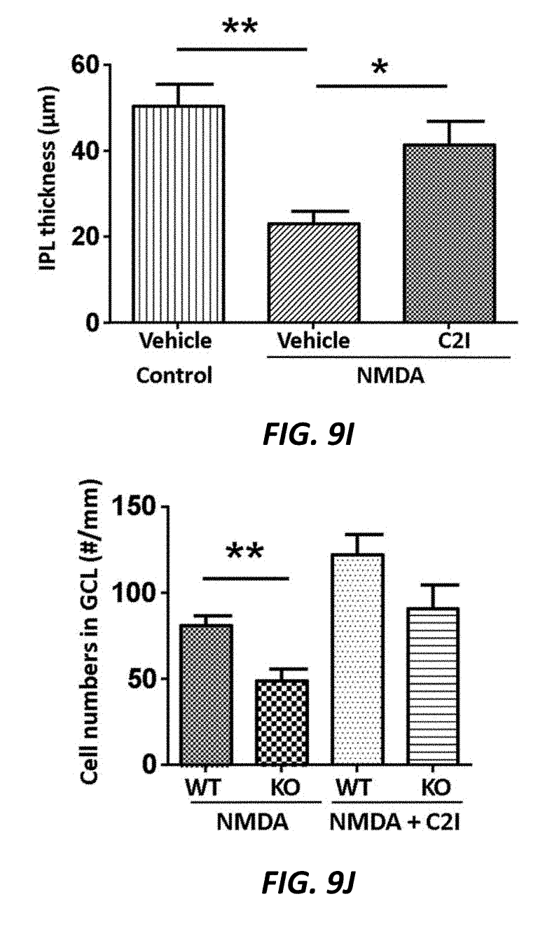

Six days after intravitreal injection of NMDA or PBS to WT mice, frozen retinal sections were prepared and H&E staining was performed to evaluate cell densities in the ganglion cell layer (GCL) and the thickness of the Inner Plexiform Layer (IPL), which contains RGC dendrites. NMDA injection (NMDA plus Vehicle) significantly reduced cell density in the GCL and IPL thickness, while PBS injection (PBS plus Vehicle) had no effect on these parameters (New FIG. 9D). Systemic injection of C2l 30 min before and 6 h after NMDA injection significantly suppressed the reduction in GCL cell density and IPL thickness (New FIG. 9E-F), suggesting that calpain-2 activation contributes to NMDA-induced cell death in GCL.

In calpain-1 KO mice, GCL cell density and IPL thickness were not affected by vehicle injection. However, the effects of NMDA injection on GCL cell density and IPL thickness were larger than in WT mice (compare New FIG. 9D with New FIG. 9G). GCL cell death in calpain-1 KO mice after NMDA injection was significantly more severe than that in WT mice (Compare New FIG. 9H with FIG. 9J), suggesting that calpain-1 supports cell survival in GCL after NMDA injection. Systemic injection of C2l to calpain-1 KO mice partially but significantly reversed NMDA-induced decrease in GCL cell density and IPL thickness (FIGS. 8I and 8J, respectively). The effect of C2l on GCL cell survival was lower in KO mice than in WT mice (FIG. 9J), further supporting the pro-survival role of calpain-1 in NMDA-induced excitotoxic insults in retina.

### Example 10

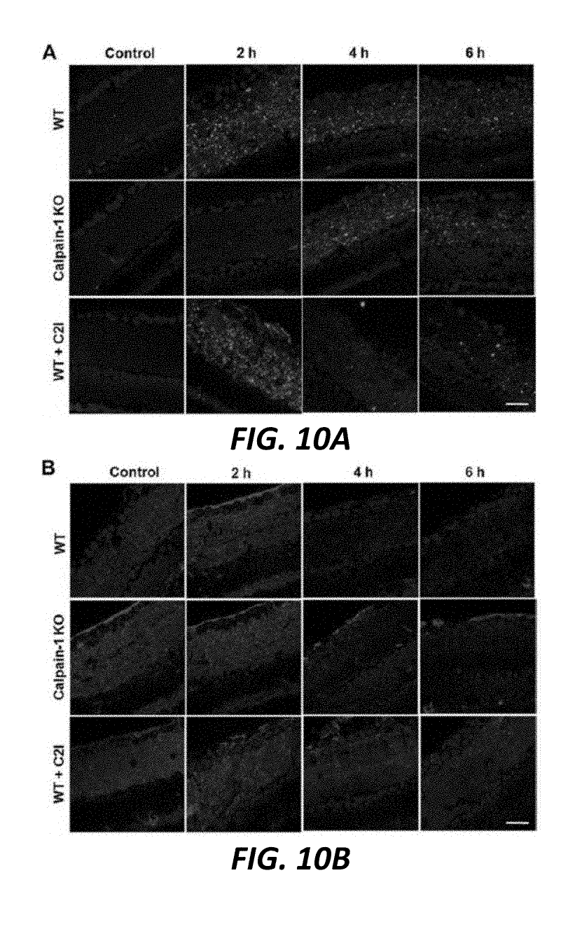

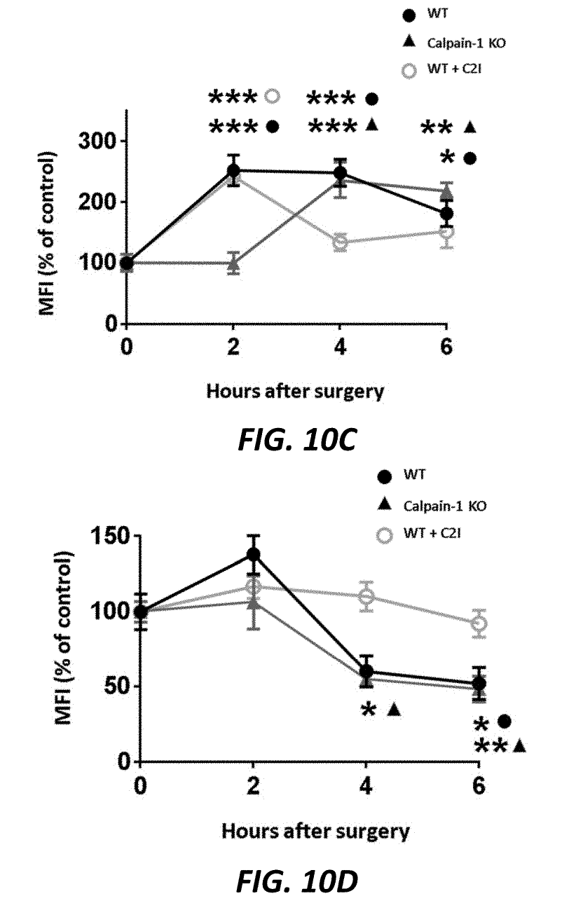

Sequential activation of calpain-1 and calpain-2 in retina after acute IOP elevation. The following IOP elevation studies were performed using a model of acute angle closure glaucoma consisting of increasing intraocular pressure (IOP) to 110 mm Hg for 60 min by inserting a needle connected to an elevated reservoir of saline into the anterior chamber. This model reproduced several features of acute angle closure, including, ischemia of retina and iris as noted by absence of red reflex and pupillary response to light. Anterior chamber synechae, resulting in a narrow angle and adhesions between the iris and the cornea, increased cells and flare in the anterior chamber and increased corneal thickness due to corneal edema. Some of these changes persisted over 3 days of observation. Eyes were collected at 0, 2, 4 and 6 h after IOP elevation and retinal frozen sections were prepared and processed for immunohistochemistry with SBDP antibody. In WT mice, SBDP was clearly present in IPL at 2, 4 and 6 h after IOP elevation. However in calpain-1 KO mice, SBDP was only evident in the IPL at 4 and 6 h but not at 2 h (FIG. 10A and C). These results suggest that calpain-2 activation is slower than calpain-1 activation in IPL after IOP elevation. To test the effect of C2l, 0.3 mg/kg of C2l was injected i.p. to WT mice at 2 h after IOP elevation. C2l injection significantly reduced SBDP signal in IPL at 4 and 6 h (FIG. 10A and C), indicating that calpain-2 activation in IPL of retina was inhibited by systemic injection of C2l. This result also suggests that calpain activity at 4 and 6 h in IPL of WT mice was mainly the result of calpain-2 activation.

To verify the time course of calpain-2 activation in retina after increased IOP, retinal sections were immunostained with an antibody against full-length PTEN, a substrate of calpain-2 but not calpain-117. In both WT and calpain-1 KO mice, PTEN-immunoreactivity in IPL was unchanged at 2 h, but was significantly reduced at 4 and 6 h after IOP elevation (FIG. 10A and D), confirming that calpain-2 was activated at 4 and 6 but not 2 h after IOP elevation. When C2l was injected to WT mice 2 h after IOP elevation, PTEN degradation at 4 and 6 h was completely blocked (FIG. 10A and D). Altogether, these results suggest that calpain-1 is briefly activated in RGC dendrites after acute IOP elevation, while calpain-2 activation in the same dendrites is delayed and prolonged.

### Example 11

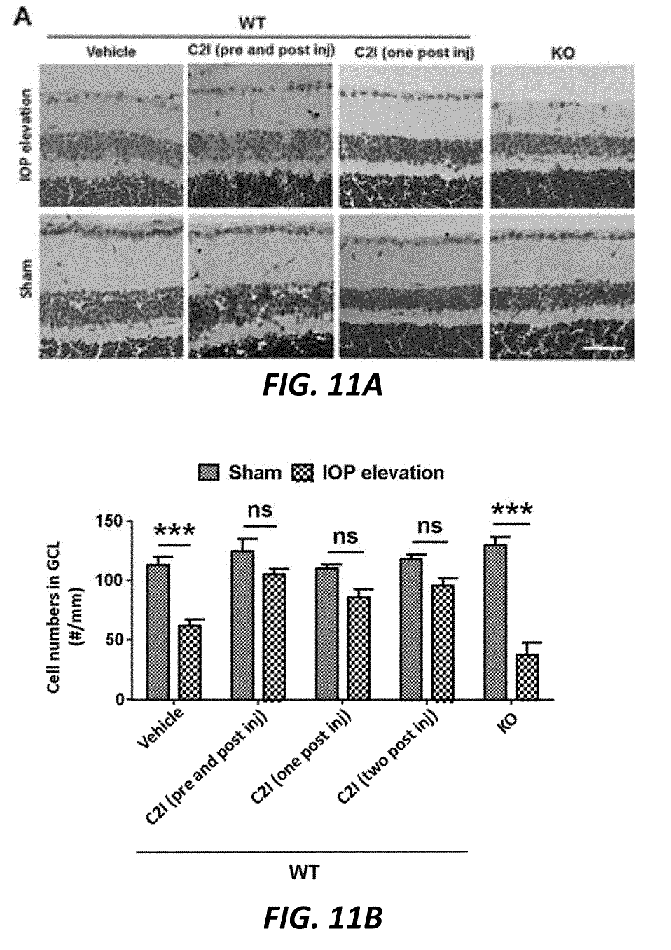

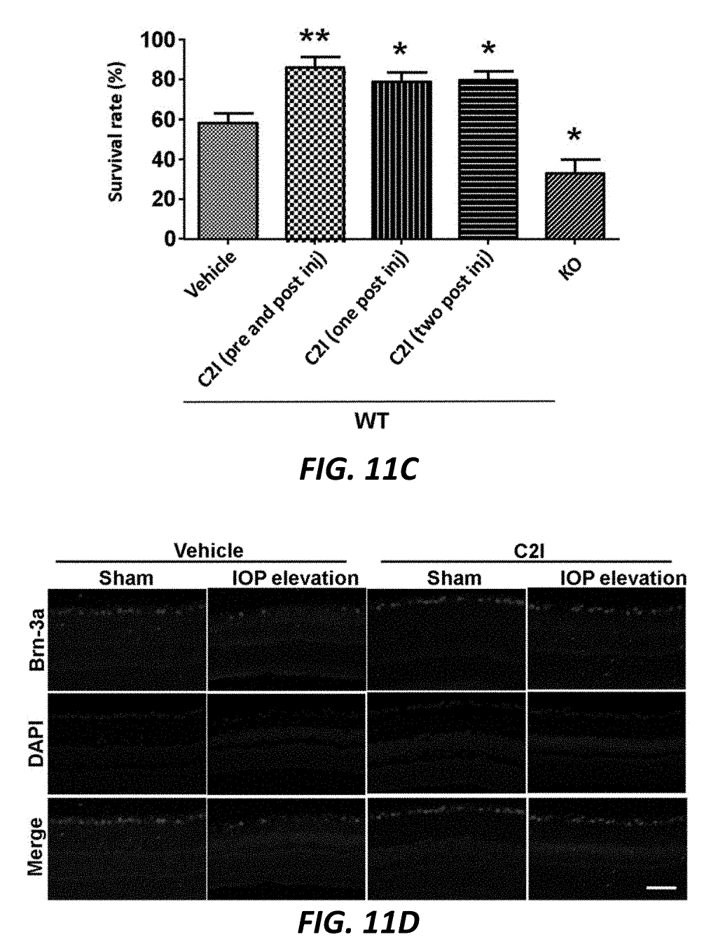

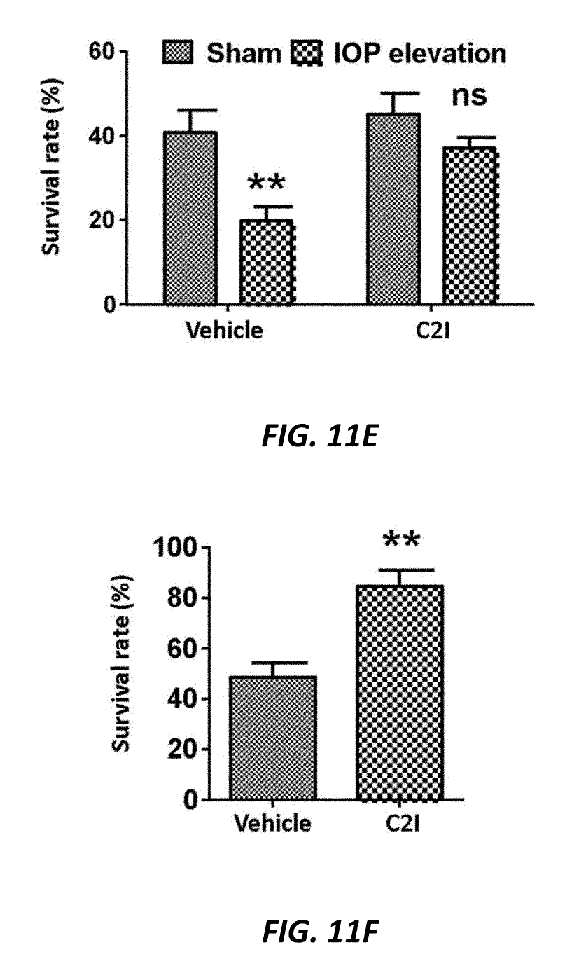

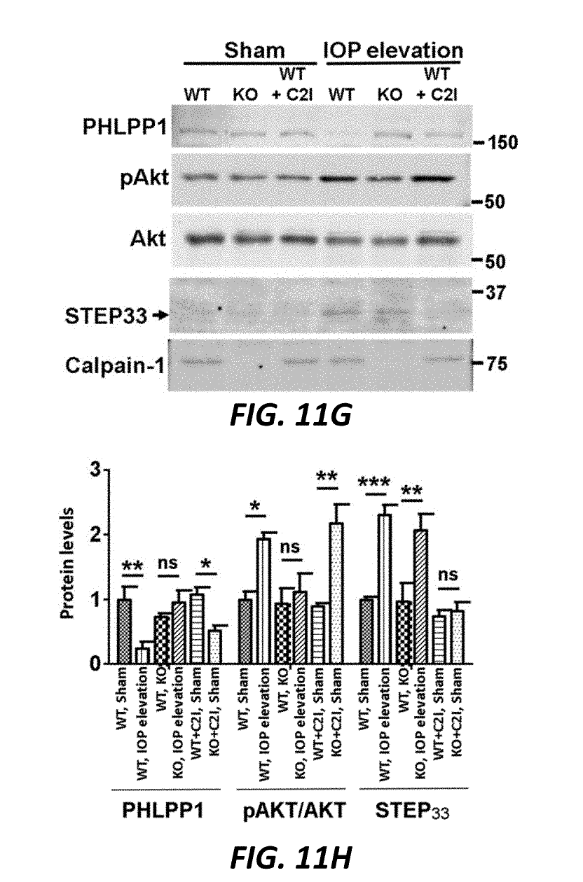

Calpain-1 and calpain-2 play opposite roles in RGC death induced by acute IOP elevation. To evaluate elevated IOP-induced retinal damage, IOP of the right eye was elevated to 110 mm Hg for 60 min, while a sham procedure was performed in the left eye. Retinal sections were collected for H&E staining 3 days after surgery (FIG. 11A and B). In WT mice injected (i.p.) with vehicle (10% DMSO in PBS), cell counts in right GCL were 62.1±5.6 cells/mm, as compared to 113.4±7.1 cells/mm in the left eye (n=7). We used three different protocols to examine the effect of C2l. First, C2l (0.3 mg/kg) was injected (i.p.) 30 min before and 2 h after acute IOP elevation (pre and post inj). Cell counts in GCL of sham eye and IOP-elevated eye were 125.1±10.5 and 105.8±4.5 cells/mm, respectively (n=3, no significant difference (ns) sham vs. IOP). Second, C2l was injected (i.p.) 2 h after IOP elevation (one post inj). Cell counts in sham and IOP-elevated eye were 110.6±3.6 and 86.4±7.0 cells/mm (n=10, ns). Third, C2l was injected (i.p.) 2 and 4 h after IOP elevation (two post inj). Cell counts in sham and IOP-elevated eye were 118.6±3.7 and 96.1±6.3 cells/mm (n=6, ns). In all three C2l injected groups, cell survival rate (ratio of cell count in IOP-elevated eye to that in sham eye) was significantly increased, as compared to vehicle-injected group (FIG. 11C). These results suggest that calpain-2 activation plays an important role in GCL cell death after IOP elevation and that C2l systemic injection has a protective effect against IOP-induced cell death.

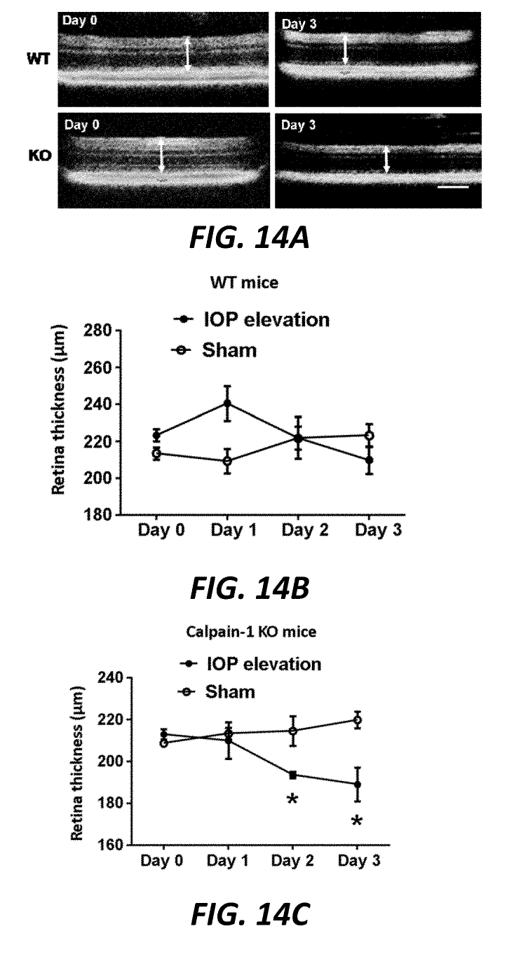

In calpain-1 KO mice, cell count in GCL of IOP-elevated eye was significantly lower than that of sham eye (37.7±10.4 vs. 130.3±7.0 cells/mm, n=4). Importantly, the cell survival rate in calpain-1 KO mice was significantly lower than that in WT mice (FIG. 11C), suggesting that calpain-1 supports cell survival in GCL after IOP elevation. To further evaluate IOP-induced retinal damage in WT and KO mice, SD-OCT was performed in IOP-elevated and sham eyes of WT mice and calpain-1 KO mice from 0 to 3 days after surgery. In general, retinal structure at day 3 was slightly different from that at day 0 in both WT and KO mice (FIG. 14A). Quantification of retinal thickness in retinal OCT images showed that retinal thickness of IOP-elevated eyes was significantly reduced at day 2 and 3, as compared to day 0 in KO mice, while the difference was not statistically significant in WT mice (FIG. 14B and C), again suggesting exacerbated retinal damage in calpain-1 KO mice, as compared to WT mice.

To specifically examine the effect of C2l on RGCs, which constitute approximately 40% of the cells in mouse GCL18, retinal sections from WT mice injected with vehicle or C2l 2 h after acute IOP elevation were immunostained with an antibody against brn-3a, a selective RGC marker19 (FIG. 11D). In WT mice injected with vehicle, RGC counts in sham eye and IOP-elevated eye were 40.9±5.2 and 19.9±3.4 cells/mm (p<0.01 sham vs. IOP, n=4). In WT mice injected with C2l, RGC counts in sham eye and IOP-elevated eye were 45.2±5.0 and 37.1±2.5 cells/mm (ns sham vs. IOP, n=5) (FIG. 11E). The survival rate of RGC with C2l injection was significantly improved, as compared to vehicle injection (FIG. 11F), suggesting that C2l systemic injection protects RGC against IOP-induced cell death.

To explore the nature of the signaling pathways downstream of calpain-1 and calpain-2, retinas in WT, calpain-1 KO and C2l-injected WT mice were collected 3 h after IOP elevation or sham surgery, homogenized and aliquots processed for Western blots (FIG. 11G-H). In WT mice, levels of PHLPP1, a phosphatase downstream of calpain-1, were significantly reduced, while levels of phospho-Akt Ser473 (pAkt), which can be dephosphorylated by PHLPP1, were significantly increased after IOP elevation. These changes in PHLPP1 and pAkt were absent in calpain-1 KO mice but present in C2l-injected WT mice, suggesting that calpain-1 but not calpain-2 mediates PHLPP1 degradation and Ak activation in retina after IOP elevation. STEP33, the product of calpain-2-mediated cleavage of striatal-enriched protein tyrosine phosphatase (STEP)13, was present in WT and calpain-1 KO mice but not in C2l-injected WT mice following increased IOP, indicating that calpain-2 but not calpain-1 mediates STEP cleavage after IOP elevation. These results suggest that both calpain-1/PHLPP1/Akt pro-survival pathway and calpain-2/STEP pro-death pathway13 are present in retina after IOP elevation, and that C2l selectively inhibits calpain-2/STEP pro-death pathway.

### Example 12

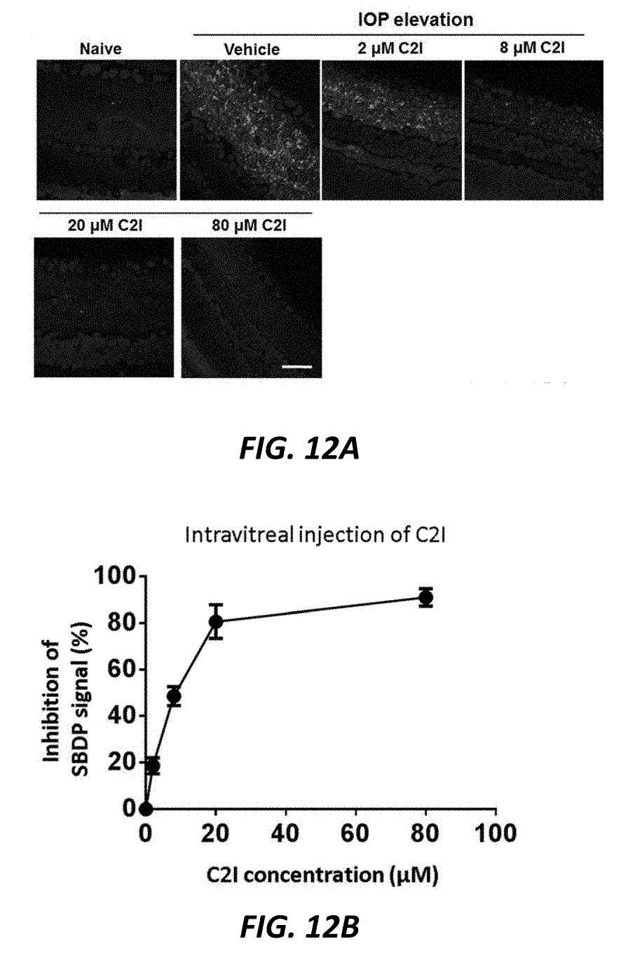

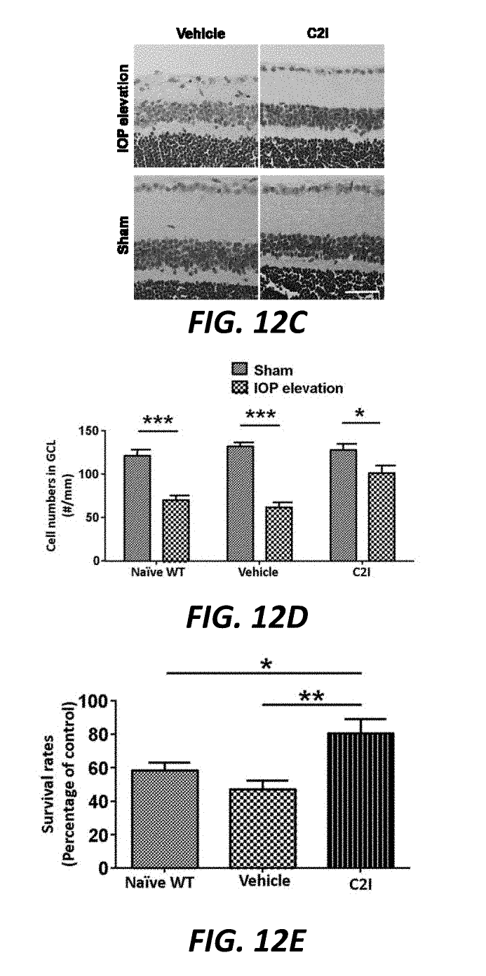

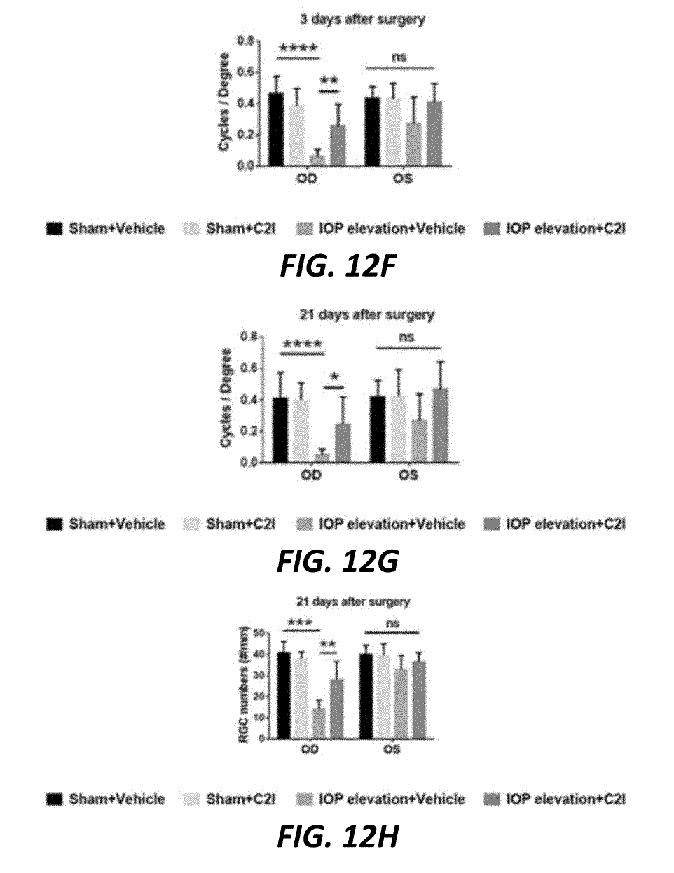

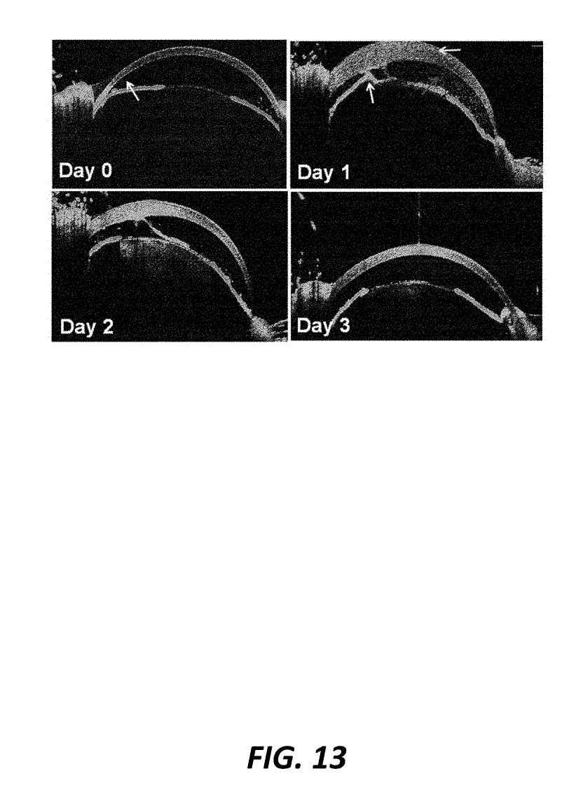

Intravitreal injection of C2l reduces cell death in GCL and prevents loss of vision caused by acute IOP elevation. We used intravitreal C2l injection in order to locally deliver C2l to retina. First, we tested the delivery efficiency by injecting different doses of C2l intravitreally 2 h after IOP elevation in calpain-1 KO mice and analyzing SBDP levels in IPL at 4 h (FIG. 12A and B). A clear dose-dependent inhibition of SBDP formation was observed, providing an apparent IC50 of 8 μM for C2l. In all subsequent experiments, 20 μM (1 μl) was used to examine the neuroprotective effects of intravitreal C2l injection. Eyes were collected 3 days after surgery for H&E staining. After vehicle injection (10% DMSO in PBS), RGC counts in sham eye and IOP-elevated eye were 132.3±4.5 and 62.0±5.7 cells/mm (p<0.001 sham vs. IOP, n=4). In C2l-treated eyes, RGC counts in sham eye and IOP-elevated eyes were 128.1±7.2 and 101.3±9.2 cells/mm (no significant difference, sham vs. IOP, n=5) (FIG. 12C and D). Survival rate with C2l injection was significantly improved, as compared to vehicle injection (80.8±8.4% vs. 47.2±5.4%, p<0.01) (FIG. 5e), suggesting that intravitreal C2l injection 2 h after IOP elevation is neuroprotective against IOP-induced cell death in GCL.

To examine vision of mice after glaucoma, we tested the optokinetic reflex (OKR) in mice (FIG. 15A). OKR is the saccadic eye movement triggered by the movement of gratings in front of the mouse eye. Changing the frequency of gratings and determining the lowest frequency triggering OKR, allows analyzing visual acuity of each eye. Acute IOP elevation or sham surgery was performed in the right eye (OD). Left eye (OS) served as a naive control. Intravitreal C2l or vehicle injection was performed 2 h after surgery. Three and 21 days after surgery, OKR was determined in both eyes (FIG. 5f,g). Visual acuity of sham eye with vehicle injection was 0.47±0.11 cpd (mean±SEM, n=7) at day 3 and 0.41±0.16 (n=7) at day 21, in good agreement with published results20,21. Visual acuity was dramatically reduced after increased IOP at both time points, which was significantly improved by C2l injection. C2l injection in the sham eye did not affect visual acuity, as compared to vehicle injection. Mice were sacrificed after OKR test at day 21 and RGC densities were analyzed with brn-3a immunostaining in retinal sections (FIG. 12H). As expected, RGC densities were significantly reduced in IOP-elevated eyes with vehicle injection, but recovered in IOP elevated eyes with C2l injection. Moreover, visual acuity was highly correlated with the number of surviving RGCs (FIG. 15B), further supporting the prominent role of calpain-2 in triggering RGC death after increased IOP.

### Example 13

Diastereoisomers of Formula 1 have profoundly different Inhibitory activities.

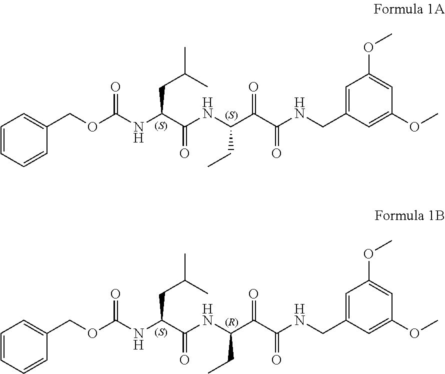

Formula 1A, where chiral center 1 is the S-form and chiral center 2 is the S-form was separated from the S-R-form (Formula 1B) using methods that are well-known methods for separating diastereoisomers. Formula 1A, which is also referred to herein as compound 18A, in this example was introduced at various concentrations into an in vitro mix comprising succinic-Leu-Tyr-AMC and μ-calpain or m-calpain (Sasaki et al, 1984), and the kinetics of the loss of fluorescence were determined for each of the calpains. The Ki of Formula 1A for μ-calpain was determined to be 181±73 nM for calpain-1, and the Ki for calpain-2 was determined to be 7.8±2.5 nM (see Table I). In contrast, the Ki of Formula 18 (also referred to herein as compound 188) for calpain-1 was determined herein to be 514±151 μM and the Ki for calpain-2 was determined to be 15.6±9.2 μM. This represents an unexpected 2000-fold difference in the activity between the two diasteriomers with respect to the inhibition of calpain-2.

TABLE 1

| | | | | Ratio |
|---|---|---|---|---|
| Calpain-2 | | | Previous | KiCalpain-1/ |
| (n = 3 − 4) | IC50 | Ki | Value * | KiCalpain-2 |
| C18 | 209.6 ± | 21.3 nM | 25 nM | 31.1 |
| | 45.6 nM | | | |
| C18A | 193.1 ± | 7.8 ± | | 23.2 |
| | 62.4 nM | 2.5 nM | | |
| C18B | 19.4 ± | 15.6 ± | | 34 |
| | 11.5 μM | 9.2 μM | | |

| Calpain-1 | | | Previous |
|---|---|---|---|
| (n = 3 − 5) | IC50 | Ki | Value |
| C18 | 910 ± | 662 ± | 940 |
| | 388 nM | 351 nM | |
| C18A | 379 ± | 181 ± | |
| | 80 nM | 73 nM | |
| C18B | 569 ± | 514 ± | |
| | 167 μM | 151 μM | |

### REFERENCES

Almasieh, M., Wilson, A. M., Morquette, B., Cueva Vargas, J. L. & Di Polo, A. (2012) The molecular basis of retinal ganglion cell death in glaucoma. Prog. Retin. Eye Res. 31: 152-181
Anderson E R, Boyle J, Zink W E, Persidsky Y, Gendelman H E, Xiong H (2003) Hippocampal synaptic dysfunction in a murine model of human immunodeficiency virus type 1 encephalitis. Neuroscience 118: 359-369
Baudry M, Chou M M, Bi X (2013) Targeting calpain in synaptic plasticity. Expert opinion on therapeutic targets 17: 579-592
Baudry M, Kramar E, Xu X, Zadran H, Moreno S, Lynch G, Gall C, Bi X (2012) Ampakines promote spine actin polymerization, long-term potentiation, and learning in a mouse model of Angelman syndrome. Neurobiology of disease 47: 210-215
Bertrand Y, Currie J C, Demeule M, Regina A, Che C, Abulrob A, Fatehi D, Sartelet H, Gabathuler R, Castaigne J P, Stanimirovic D, Beliveau R (2010) Transport characteristics of a novel peptide platform for CNS therapeutics. Journal of cellular and molecular medicine 14: 2827-2839
Briz, V., et al. (2013) Calpain-2-mediated PTEN degradation contributes to BDNF-induced stimulation of dendritic protein synthesis. J. Neurosci. 33: 4317-4328
Cahill, H. & Nathans, J. (2008) The optokinetic reflex as a tool for quantitative analyses of nervous system function in mice: application to genetic and drug-induced variation. PLoS One 3: e2055
Cameron, D. J., et al. (2013) The optokinetic response as a quantitative measure of visual acuity in zebrafish. J. Vis. Exp. 80: e50832
Capocchi G, Zampolini M, Larson J (1992) Theta burst stimulation is optimal for induction of LTP at both apical and basal dendritic synapses on hippocampal CA1 neurons. Brain research 591: 332-336
Chi, W., et al. (2014) Caspase-8 promotes NLRP1/NLRP3 inflammasome activation and IL-1beta production in acute glaucoma. Proc. Natl. Acad. Sci. USA 111: 11181-11186
Chiu, K., Lam, T. T., Ying Li, W. W., Caprioli, J. & Kwong Kwong, J. M. (2005) Calpain and N-methyl-d-aspartate (NMDA)-induced excitotoxicity in rat retinas. Brain Res. 1046: 207-215
Chou J S, Impens F, Gevaert K, Davies P L (2011) m-Calpain activation in vitro does not require autolysis or subunit dissociation. Biochimica et biophysica acta 1814: 864-872
Clackson T, Hoogenboom H R, Griffiths A D, Winter G (1991) Making antibody fragments using phage display libraries. Nature 352: 624-628
Congdon, N., Wang, F. & Tielsch, J. M. (1992) Issues in the epidemiology and population-based screening of primary angle-closure glaucoma. Surv. Ophthalmol. 36: 411-423
Cuerrier D, Moldoveanu T, Campbell R L, Kelly J, Yoruk B, Verhelst S H, Greenbaum D, Bogyo M, Davies P L (2007) Development of calpain-specific inactivators by screening of positional scanning epoxide libraries. The Journal of biological chemistry 282: 9600-9611
Cuerrier D, Moldoveanu T, Inoue J, Davies P L, Campbell R L (2006) Calpain inhibition by alpha-ketoamide and cyclic hemiacetal inhibitors revealed by X-ray crystallography. Biochemistry 45: 7446-7452
Cwirla S E, Peters E A, Barrett R W, Dower W J (1990) Peptides on phage: a vast library of peptides for identifying ligands. Proceedings of the National Academy of Sciences of the United States of America 87: 6378-6382
del Cerro S, Larson J, Oliver M W, Lynch G (1990) Development of hippocampal long-term potentiation is reduced by recently introduced calpain inhibitors. Brain research 530: 91-95
Denny J B, Polan-Curtain J, Ghuman A, Wayner M J, Armstrong D L (1990) Calpain inhibitors block long-term potentiation. Brain research 534: 317-320
Donkor I O (2011) Calpain inhibitors: a survey of compounds reported in the patent and scientific literature. Expert opinion on therapeutic patents 21: 601-636
Douglas, R. M., et al. (2005) Independent visual threshold measurements in the two eyes of freely moving rats and mice using a virtual-reality optokinetic system. Vis. Neurosci. 22: 677-684
Dufes C, Robaian M A, Somani S (2013) Transferrin and the transferrin receptor for the targeted delivery of therapeutic agents to the brain and cancer cells. Therapeutic delivery 4: 629-640
Farkas B, Tantos A, Schlett K, Vilagi I, Friedrich P (2004) Ischemia-induced increase in long-term potentiation is warded off by specific calpain inhibitor PD150606. Brain research 1024: 150-158
Gabathuler R (2009) Blood-brain barrier transport of drugs for the treatment of brain diseases. CNS & neurological disorders drug targets 8: 195-204
Gabathuler R (2010a) Approaches to transport therapeutic drugs across the blood-brain barrier to treat brain diseases. Neurobiology of disease 37: 48-57
Gabathuler R (2010b) Development of new peptide vectors for the transport of therapeutic across the blood-brain barrier. Therapeutic delivery 1: 571-586
Goll D E, Thompson V F, Li H, Wei W, Cong J (2003) The calpain system. Physiological reviews 83: 731-801
Good M C, Zalatan J G, Lim W A (2011) Scaffold proteins: hubs for controlling the flow of cellular information. Science 332: 680-686
Greene R (2001) Circuit analysis of NMDAR hypofunction in the hippocampus, in vitro, and psychosis of schizophrenia. Hippocampus 11: 569-577
Hauss-Wegrzyniak B, Lynch M A, Vraniak P D, Wenk G L (2002) Chronic brain inflammation results in cell loss in the entorhinal cortex and impaired LTP in perforant path-granule cell synapses. Experimental neurology 176: 336-341
Hosfield C M, Elce J S, Davies P L, Jia Z (1999) Crystal structure of calpain reveals the structural basis for Ca(2+)-dependent protease activity and a novel mode of enzyme activation. The EMBO journal 18: 6880-6889
Howland J G, Wang Y T (2008) Synaptic plasticity in learning and memory: stress effects in the hippocampus. Progress in brain research 169: 145-158
Jeon, C. J., Strettoi, E. & Masland, R. H. (1998)The major cell populations of the mouse retina. J. Neurosci. 18: 8936-8946
Kang B S, Cooper D R, Devedjiev Y, Derewenda U, Derewenda Z S (2003) Molecular roots of degenerate specificity in syntenin's PDZ2 domain: reassessment of the PDZ recognition paradigm. Structure 11: 845-853
Keblesh J P, Dou H, Gendelman H E, Xiong H (2009) 4-Aminopyridine improves spatial memory in a murine model of HIV-1 encephalitis. Journal of neuroimmune pharmacology: the official journal of the Society on NeuroImmune Pharmacology 4: 317-327
Kim J J, Song E Y, Kosten T A (2006) Stress effects in the hippocampus: synaptic plasticity and memory. Stress 9: 1-11
Kleschevnikov A M, Belichenko P V, Faizi M, Jacobs L F, Htun K, Shamloo M, Mobley W C (2012) Deficits in cognition and synaptic plasticity in a mouse model of Down syndrome ameliorated by GABAB receptor antagonists. The Journal of neuroscience: the official journal of the Society for Neuroscience 32: 9217-9227
Kohda K, Harada K, Kato K, Hoshino A, Motohashi J, Yamaji T, Morinobu S, Matsuoka N, Kato N (2007) Glucocorticoid receptor activation is involved in producing abnormal phenotypes of single-prolonged stress rats: a putative post-traumatic stress disorder model. Neuroscience 148: 22-33
Kornau H C, Schenker L T, Kennedy M B, Seeburg P H (1995) Domain interaction between NMDA receptor subunits and the postsynaptic density protein PSD-95. Science 269: 1737-1740
Lasley S M, Gilbert M E (2000) Glutamatergic components underlying lead-induced impairments in hippocampal synaptic plasticity. Neurotoxicology 21: 1057-1068
Li S T & Ju J G (2012) Functional roles of synaptic and extrasynaptic NMDA receptors in physiological and pathological neuronal activities. Current drug targets 13: 207-221
Li Z, Ortega-Vilain A C, Patil G S, Chu D L, Foreman J E, Eveleth D D, Powers J C (1996) Novel peptidyl alpha-keto amide inhibitors of calpains and other cysteine proteases. Journal of medicinal chemistry 39: 4089-4098
Li Z, Patil G S, Golubski Z E, Hori H, Tehrani K, Foreman J E, Eveleth D D, Bartus R T, Powers J C (1993) Peptide alpha-keto ester, alpha-keto amide, and alpha-keto acid inhibitors of calpains and other cysteine proteases. Journal of medicinal chemistry 36: 3472-3480
Lynch G, Kramar E A, Rex C S, Jia Y, Chappas D, Gall C M, Simmons D A (2007) Brain-derived neurotrophic factor restores synaptic plasticity in a knock-in mouse model of Huntington's disease. The Journal of neuroscience: the official journal of the Society for Neuroscience 27: 4424-4434
Miyazaki S, Katayama Y, Lyeth B G, Jenkins L W, DeWitt D S, Goldberg S J, Newlon P G, Hayes R L (1992) Enduring suppression of hippocampal long-term potentiation following traumatic brain injury in rat. Brain research 585: 335-339
Moretti P, Levenson J M, Battaglia F, Atkinson R, Teague R, Antalffy B, Armstrong D, Arancio O, Sweatt J D, Zoghbi H Y (2006) Learning and memory and synaptic plasticity are impaired in a mouse model of Rett syndrome. The Journal of neuroscience: the official journal of the Society for Neuroscience 26: 319-327
Nadal-Nicolas, F. M., et al. (2009) Brn3a as a marker of retinal ganglion cells: qualitative and quantitative time course studies in naive and optic nerve-injured retinas. Invest. Ophthalmol. Vis. Sci. 50: 3860-3868
Nakajima, E., David, L. L., Bystrom, C., Shearer, T. R. & Azuma, M. (2006) Calpain-specific proteolysis in primate retina: Contribution of calpains in cell death. Invest. Ophthalmol. Vis. Sci. 47: 5469-5475
Nimmrich V, Reymann K G, Strassburger M, Schoder U H, Gross G, Hahn A, Schoemaker H, Wicke K, Moller A (2010) Inhibition of calpain prevents NMDA-induced cell death and beta-amyloid-induced synaptic dysfunction in hippocampal slice cultures. British journal of pharmacology 159: 1523-1531
Nucci, C., et al. Neurochemical evidence to implicate elevated glutamate in the mechanisms of high intraocular pressure (IOP)-induced retinal ganglion cell death in rat. Neurotoxicology 26, 935-941 (2005)
Oka, T., Tamada, Y., Nakajima, E., Shearer, T. R. & Azuma, M. (2006) Presence of calpain-induced proteolysis in retinal degeneration and dysfunction in a rat model of acute ocular hypertension. J. Neurosci. Res. 83: 1342-1351
Pal G P, De Veyra T, Elce J S, Jia Z (2003) Crystal structure of a micro-like calpain reveals a partially activated conformation with low Ca2+ requirement. Structure 11: 1521-1526
Park S H, Zarrinpar A, Lim W A (2003) Rewiring MAP kinase pathways using alternative scaffold assembly mechanisms. Science 299: 1061-1064
Park, S. W., et al. (2011) A selective inhibitor of drp1, mdivi-1, increases retinal ganglion cell survival in acute ischemic mouse retina. Invest. Ophthalmol. Vis. Sci. 52: 2837-2843
Parsons, M. P. & Raymond, L. A. (2014) Extrasynaptic NMDA receptor involvement in central nervous system disorders. Neuron 82: 279-293
Powers J C, Asgian J L, Ekici O D, James K E (2002) Irreversible inhibitors of serine, cysteine, and threonine proteases. Chemical reviews 102: 4639-4750
Powers J C, Kam C M, Ricketts K M, Casillas R P (2000) Cutaneous protease activity in the mouse ear vesicant model. Journal of applied toxicology: JAT 20 Suppl 1: 5177-182
Prusky, G. T., Alam, N. M., Beekman, S. & Douglas, R. M. (2004) Rapid quantification of adult and developing mouse spatial vision using a virtual optomotor system. Invest. Ophthalmol. Vis. 45: 4611-4616
Russo, R., et al. (2011) Calpain-mediated cleavage of Beclin-1 and autophagy deregulation following retinal ischemic injury in vivo. Cell Death Dis. 2: e144
Sakamoto, Y. R., et al. (2000) Involvement of calpain isoforms in ischemia-reperfusion injury in rat retina. Curr. Eye Res. 21: 571-580
Sakamoto, K., et al. (2006) Inducible nitric oxide synthase inhibitors abolished histological protection by late ischemic preconditioning in rat retina. Exp. Eye Res. 82: 512-518
Sakatani, T. & Isa, T. (2004) PC-based high-speed video-oculography for measuring rapid eye movements in mice. Neurosci. Res. 49: 123-131
Sasaki T, Kikuchi T, Yumoto N, Yoshimura N, Murachi T (1984) Comparative specificity and kinetic studies on porcine calpain I and calpain II with naturally occurring peptides and synthetic fluorogenic substrates. The Journal of biological chemistry 259: 12489-12494
Schnyder A, Huwyler J (2005) Drug transport to brain with targeted liposomes. NeuroRx: the journal of the American Society for Experimental NeuroTherapeutics 2: 99-107
Shimazawa, M., et al. (2010) A novel calpain inhibitor, ((1S)-1-((((1S)-1-Benzyl-3-cyclopropylamino-2,3-di-oxopropyl)amino)carbonyl)-3-methylbutyl)carbamic acid 5-methoxy-3-oxapentyl ester (SNJ-1945), reduces murine retinal cell death in vitro and in vivo. J. Pharmacol. Exp. Ther. 332: 380-387
Shipton O A, Leitz J R, Dworzak J, Acton C E, Tunbridge E M, Denk F, Dawson H N, Vitek M P, Wade-Martins R, Paulsen O, Vargas-Caballero M (2011) Tau protein is required for amyloid {beta}-induced impairment of hippocampal long-term potentiation. The Journal of neuroscience: the official journal of the Society for Neuroscience 31: 1688-1692
Shiraha H, Glading A, Chou J, Jia Z, Wells A (2002) Activation of m-calpain (calpain II) by epidermal growth factor is limited by protein kinase A phosphorylation of m-calpain. Molecular and cellular biology 22: 2716-2727
Sorimachi H, Mamitsuka H, Ono Y (2012) Understanding the substrate specificity of conventional calpains. Biological chemistry 393: 853-871
Stabach P R, Cianci C D, Glantz S B, Zhang Z, Morrow J S (1997) Site-directed mutagenesis of alpha II spectrin at codon 1175 modulates its mu-calpain susceptibility. Biochemistry 36: 57-65
Staubli U, Larson J, Thibault O, Baudry M, Lynch G (1988) Chronic administration of a thiol-proteinase inhibitor blocks long-term potentiation of synaptic responses. Brain research 444: 153-158
Takeda A (2012) Zinc signaling in the hippocampus and its relation to pathogenesis of depression. Journal of trace elements in medicine and biology: organ of the Society for Minerals and Trace Elements 26: 80-84
Urtti, A. (2006) Challenges and obstacles of ocular pharmacokinetics and drug delivery. Adv. Drug Deliv. Rev. 58: 1131-1135
Wang, N., Wu, H. & Fan, Z. (2002) Primary angle closure glaucoma in Chinese and Western populations. Chin. Med. J. (Engl) 115: 1706-1715
Wang, L., Cioffi, G. A., Cull, G., Dong, J. & Fortune, B. (2002) Immunohistologic evidence for retinal glial cell changes in human glaucoma. Invest. Ophthalmol. Vis. Sci. 43: 1088-1094
Wang, Y., Briz, V., Chishti, A., Bi, X. & Baudry, M. (2013) Distinct roles for mu-calpain and m-calpain in synaptic NMDAR-mediated neuroprotection and extrasynaptic NMDAR-mediated neurodegeneration. J. Neurosci. 33: 18880-18892
Wang, Y., et al. (2014) A molecular brake controls the magnitude of long-term potentiation. Nat. Commun. 5: 3051
Wiescholleck V, Manahan-Vaughan D (2013) Persistent deficits in hippocampal synaptic plasticity accompany losses of hippocampus-dependent memory in a rodent model of psychosis. Frontiers in integrative neuroscience 7: 12
Woods D F, Bryant P J (1991) The discs-large tumor suppressor gene of Drosophila encodes a guanylate kinase homolog localized at septate junctions. Cell 66: 451-464
Xie Y, Wang Y, Zhang T, Ren G, Yang Z (2012) Effects of nanoparticle zinc oxide on spatial cognition and synaptic plasticity in mice with depressive-like behaviors. Journal of biomedical science 19: 14
Xu W, Wong T P, Chery N, Gaertner T, Wang Y T, Baudry M (2007) Calpain-mediated mGluR1alpha truncation: a key step in excitotoxicity. Neuron 53: 399-412
Xu, J., et al. (2009) Extrasynaptic NMDA receptors couple preferentially to excitotoxicity via calpain-mediated cleavage of STEP. J. Neurosci. 29: 9330-9343
Zadran S, Jourdi H, Rostamiani K, Qin Q, Bi X, Baudry M (2010) Brain-derived neurotrophic factor and epidermal growth factor activate neuronal m-calpain via mitogen-activated protein kinase-dependent phosphorylation. The Journal of neuroscience: the official journal of the Society for Neuroscience 30: 1086-1095

Sequences:

PTEN Homo Sapiens, SEQ ID NO 1:
MTAIIKEIVSRNKRRYQEDGFDLDLTYIYPNIIAMGFPAERLEGVYRNNIDDVVRFLDSKH
KNHYKIYNLCAERHYDTAKFNCRVAQYPFEDHNPPQLELIKPFCEDLDQWLSEDDNHV
AAIHCKAGKGRTGVMICAYLLHRGKFLKAQEALDFYGEVRTRDKKGVTIPSQRRYVYY
YSYLLKNHLDYRPVALLFHKMMFETIPMFSGGTCNPQFVVCQLKVKIYSSNSGPTRRE
DKFMYFEFPQPLPVCGDIKVEFFHKQNKMLKKDKMFHFWVNTFFIPGPEETSEKVENG
SLCDQEIDSICSIERADNDKEYLVLTLTKNDLDKANKDKANRYFSPNFKVKLYFTKTVEE
PSNPEASSSTSVTPDVSDNEPDHYRYSDTTDSDPENEPFDEDQHTQITKV
μ-CALPAIN Homo Sapiens, Seq ID NO 2:
MSEEIITPVYCTGVSAQVQKQRARELGLGRHENAIKYLGQDYEQLRVRCLQSGTLFRD
EAFPPVPQSLGYKDLGPNSSKTYGIKWKRPTELLSNPQFIVDGATRTDICQGALGDCW
LLAAIASLTLNDTLLHRVVPHGQSFQNGYAGIFHFQLWQFGEWVDVVVDDLLPIKDGKL
VFVHSAEGNEFWSALLEKAYAKVNGSYEALSGGSTSEGFEDFIGGVTEWYELRKAPS
DLYQIILKALERGSLLGCSIDISSVLDMEAITFKKLVKGHAYSVTGAKQVNYRGQVVSLIR
MRNPWGEVEWTGAWSDSSSEWNNVDPYERDQLRVKMEDGEFWMSFRDFMREFTR
LEICNLTPDALKSRTIRKWNTTLYEGTWRRGSTAGGCRNYPATFWVNPQFKIRLDETD
DPDDYGDRESGCSFVLALMQKHRRRERRFGRDMETIGFAVYEVPPELVGQPAVHLKR
DFFLANASRARSEQFINLREVSTRFRLPPGEYVVVPSTFEPNKEGDFVLRFFSEKSAGT
VELDDQIQANLPDEQVLSEEEIDENFKALFRQLAGEDMEISVKELRTILNRIISKHKDLRT
KGFSLESCRSMVNLMDRDGNGKLGLVEFNILWNRIRNYLSIFRKFDLDKSGSMSAYEM
RMAIESAGFKLNKKLYELIITRYSEPDLAVDFDNFVCCLVRLETMFRFFKTLDTDLDGVV
TFDLFKWLQLTMFA
μ-CALPAIN Mus Musculus, SEQ ID NO 3:
MTEELITPVYCTGVSAQVQKKRDKELGLGRHENAIKYLGQDYETLRARCLQSGVLFQD
EAFPPVSHSLGFKELGPHSSKTYGIKWKRPTELMSNPQFIVDGATRTDICQGALGDCW
LLAAIASLTLNETILHRVVPYGQSFQDGYAGIFHFQLWQFGEWVDVVIDDLLPTKDGKL
VFVHSAQGNEFWSALLEKAYAKVNGSYEALSGGCTSEAFEDFTGGVTEWYDLQKAPS
DLYQIILKALERGSLLGCSINISDIRDLEAITFKNLVRGHAYSVTGAKQVTYQGQRVNLIR
MRNPWGEVEWKGPWSDSSYEWNKVDPYEREQLRVKMEDGEFWMSFRDFIREFTKL
EICNLTPDALKSRTLRNWNTIFYEGTWRRGSTAGGCRNYPATFWVNPQFKIRLEEVD
DADDYDNRESGCSFLLALMQKHRRRERRFGRDMETIGFAVYQVPRELAGQPVHLKR
DFFLANASRAQSEHFINLREVSNRIRPPPGEYIVVPSTFEPNKEGDFLLRFFSEKKAGT
QELDDQIQANLPDEKVLSEEEIDDNFKTLFSKLAGDDMEISVKELQTILNRIISKHKDLRT
NGFSLESCRSMVNLMDRDGNGKLGLVEFNILWNRIRNYLTIFRKFDLDKSGSMSAYEM
RMAIEAAGFKLNKKLHELIITRYSEPDLAVDFDNFVCCLVRLETMFRFFKLLDTDLNGVV
TFDLFKWLQLTMFA
μ-calpain, Bos Taurus. SEQ ID NO: 4
MAEEFITPVYCTGVSAQVQKQRAKELGLGRHENAIKYLGQDYEQLRVHCLQRGALFR
DEAFPPVPQSLGFKELGPNSSKTYGIKWKRPTELFSNPQFIVDGATRTDICQGALGDC
WLLAAIASLTLNDTLLHRVVPHGQSFQDGYAGIFHFQLWQFGEWVDVVVDDLLPTKDG
KLVFVHSAQGNEFWSALLEKAYAKVNGSYEALSGGSTSEGFEDFIGGVTEWYELRKA
PSDLYNIILKALERGSLLGCSIDISSILDMEAVTFKKLVKGHAYSVTGAKQVNYQGQMVN
LIRMRNPWGEVEWTGAWSDGSSEWNGVDPYMREQLRVKMEDGEFWMSFRDFMRE
FTRLEICNLTPDALKSQRFRNWNTTLYEGTWRRGSTAGGCRNYPATFWVNPQFKIRL
EETDDPDPDDYGGRESGCSFLLALMQKHRRRERRFGRDMETIGFAVYEVPPELMGQ
PAVHLKRDFFLSNASRARSEQFINLREVSTRFRLPPGEYVVVPSTFEPNKEGDFVLRFF
SEKSAGTQELDDQVQANLPDEQVLSEEEIDENFKSLFRQLAGEDMEISVKELRTILNRII
SKHKDLRTTGFSLESCRSMVNLMDRDGNGKLGLVEFNILWNRIRNYLSIFRKFDLDKS
GSMSAYEMRMAIEFAGFKLNKKLYELIITRYSEPDLAVDFDNFVCCLVRLETMFRFFKT
LDTDLDGVVTFDLFKWLQLTMFA
μ-calpain, Rattus norvegicus. SEQ ID NO: 5
MAEELITPVYCTGVSAQVQKQRDKELGLGRHENAIKYLGQDYENLRARCLQNGVLFQ
DDAFPPVSHSLGFKELGPNSSKTYGIKWKRPTELLSNPQFIVDGATRTDICQGALGDC
WLLAAIASLTLNETILHRVVPYGQSFQEGYAGIFHFQLWQFGEWVDVVVDDLLPTKDG
KLVFVHSAQGNEFWSALLEKAYAKVNGSYEALSGGCTSEAFEDFTGGVTEWYDLQKA
PSDLYQIILKALERGSLLGCSINISDIRDLEAITFKNLVRGHAYSVTDAKQVTYQGQRVNL
IRMRNPWGEVEWKGPWSDNSYEWNKVDPYEREQLRVKMEDGEFWMSFRDFIREFT
KLEICNLTPDALKSRTLRNWNTTFYEGTWRRGSTAGGCRNYPATFWVNPQFKIRLEEV
DDADDYDSRESGCSFLLALMQKHRRRERRFGRDMETIGFAVYQVPRELAGQPVHLKR
DFFLANASRAQSEHFINLREVSNRIRLPPGEYIVVPSTFEPNKEGDFLLRFFSEKKAGT
QELDDQIQANLPDEKVLSEEEIDDNFKTLFSKLAGDDMEISVKELQTILNRIISKHKDLRT
NGFSLESCRSMVNLMDRDGNGKLGLVEFNILWNRIRNYLTIFRKFDLDKSGSMSAYEM
RMAIEAAGFKLNKKLHELIITRYSEPDLAVDFDNFVCCLVRLETMFRFFKILDTDLDGVV
TFDLFKWLQLTMFA
μ-calpain, Sus Scrofa. SEQ ID NO: 6
MAEEVITPVYCTGVSAQVQKLRAKELGLGRHENAIKYLGQDYEQLRAHCLQSGSLFRD
EAFPPVPQSLGFKELGPNSSKTYGVKWKRPTELFSNPQFIVDGATRTDICQGALGDC
WLLAAIASLTLNDTLLHRVVPHGQSFQNGYAGIFHFQLWQFGEWVDVVVDDLLPTKDG
KLVFVHSAQGNEFWSALLEKAYAKVNGSYEALSGGSTSEGFEDFTGGVTEWYELRKA
PSDLYSIILKALERGSLLGCSIDISSVLDMEAVTFKKLVKGHAYSVTGAKQVNYQGQMV
NLIRMRNPWGEVEWTGAWSDGSSEWNGVDPYQRDQLRVRMEDGEFWMSFRDFLR
EFTRLEICNLTPDALKSQRVRNWNTTLYEGTWRRGSTAGGCRNYPATFWVNPQFKIR
LEETDDPEDDYGGRESGCSFVLALMQKHRRRERRFGRDMETIGFAVYEVPPELVGQP
VHLKRDFFLANASRARSEQFINLREVSTRFRLPPGEYVVVPSTFEPNKEGDFVLRFFSE
KKAGTQELDDQVQAILPDEQVLSEEEIDENFKALFRQLAGEDMEISVRELRTILNRIISKH
KDLRTKGFSLESCRSMVNLMDRDGNGKLGLVEFNILWNRIRNYLSIFRKFDLDKSGSM
SAYEMRMAIESAGFKLNKKLFELIITRYSEPDLAVDFDNFVCCLVRLETMFRFFKTLDTD
LDGVVTFDLFKWLQLTMFA
μ-CALPAIN fragment, Seq ID NO 7: LDTDLDGVVTFDLFKWLQLTMFA
μ-CALPAIN fragment, Seq ID NO 8: DTDLDGVVTFDLFKWLQLTMFA
μ-CALPAIN fragment, Seq ID NO 9: TDLDGVVTFDLFKWLQLTMFA
μ-CALPAIN fragment, Seq ID NO 10: DLDGVVTFDLFKWLQLTMFA
μ-CALPAIN fragment, Seq ID NO 11: LDGVVTFDLFKWLQLTMFA
μ-CALPAIN fragment, Seq ID NO 12: DGVVTFDLFKWLQLTMFA
μ-CALPAIN fragment, Seq ID NO 13: GVVTFDLFKWLQLTMFA
μ-CALPAIN fragment, Seq ID NO 14: VVTFDLFKWLQLTMFA
μ-CALPAIN fragment, Seq ID NO 15: VTFDLFKWLQLTMFA
μ-CALPAIN fragment, Seq ID NO 16: TFDLFKWLQLTMFA
μ-CALPAIN fragment, Seq ID NO 17: FDLFKWLQLTMFA
μ-CALPAIN fragment, Seq ID NO 18: DLFKWLQLTMFA
μ-CALPAIN fragment, Seq ID NO 19: LFKWLQLTMFA
μ-CALPAIN fragment, Seq ID NO 20: FKWLQLTMFA
μ-CALPAIN fragment, Seq ID NO 21: KWLQLTMFA
μ-CALPAIN fragment, Seq ID NO 22: WLQLTMFA
μ-CALPAIN fragment, Seq ID NO 23: LQLTMFA
μ-CALPAIN fragment, Seq ID NO 24: QLTMFA
μ-CALPAIN fragment, Seq ID NO 25: LTMFA
μ-CALPAIN fragment, Seq ID NO 26: TMFA
μ-CALPAIN fragment, Seq ID NO 27: LDTDLDGVVTFDLFKWLQLDMFA
μ-CALPAIN fragment, Seq ID NO 28: DTDLDGVVTFDLFKWLQLDMFA
μ-CALPAIN fragment, Seq ID NO 29: TDLDGVVTFDLFKWLQLDMFA
μ-CALPAIN fragment, Seq ID NO 30: DLDGVVTFDLFKWLQLDMFA
μ-CALPAIN fragment, Seq ID NO 31: LDGVVTFDLFKWLQLDMFA
μ-CALPAIN fragment, Seq ID NO 32: DGVVTFDLFKWLQLDMFA
μ-CALPAIN fragment, Seq ID NO 33: GVVTFDLFKWLQLDMFA
μ-CALPAIN fragment, Seq ID NO 34: VVTFDLFKWLQLDMFA
μ-CALPAIN fragment, Seq ID NO 35: VTFDLFKWLQLDMFA
μ-CALPAIN fragment, Seq ID NO 36: TFDLFKWLQLDMFA
μ-CALPAIN fragment, Seq ID NO 37: FDLFKWLQLDMFA
μ-CALPAIN fragment, Seq ID NO 38: DLFKWLQLDMFA
μ-CALPAIN fragment, Seq ID NO 39: LFKWLQLDMFA
μ-CALPAIN fragment, Seq ID NO 40: FKWLQLDMFA
μ-CALPAIN fragment, Seq ID NO 41: KWLQLDMFA
μ-CALPAIN fragment, Seq ID NO 42: WLQLDMFA
μ-CALPAIN fragment, Seq ID NO 43: LQLDMFA
μ-CALPAIN fragment, Seq ID NO 44: QLDMFA
μ-CALPAIN fragment, Seq ID NO 45: LDMFA
μ-CALPAIN fragment, Seq ID NO 46: LDTDLDGVVTFDLFKWLQLEMFA
μ-CALPAIN fragment, Seq ID NO 47: DTDLDGVVTFDLFKWLQLEMFA
μ-CALPAIN fragment, Seq ID NO 48: TDLDGWTFDLFKWLQLEMFA
μ-CALPAIN fragment, Seq ID NO 49: DLDGVVTFDLFKWLQLEMFA
μ-CALPAIN fragment, Seq ID NO 50: LDGVVTFDLFKWLQLEMFA
μ-CALPAIN fragment, Seq ID NO 51: DGVVTFDLFKWLQLEMFA
μ-CALPAIN fragment, Seq ID NO 52: GVVTFDLFKWLQLEMFA
μ-CALPAIN fragment, Seq ID NO 53: VVTFDLFKWLQLEMFA
μ-CALPAIN fragment, Seq ID NO 54: VTFDLFKWLQLEMFA
μ-CALPAIN fragment, Seq ID NO 55: TFDLFKWLQLEMFA
μ-CALPAIN fragment, Seq ID NO 56: FDLFKWLQLEMFA
μ-CALPAIN fragment, Seq ID NO 57: DLFKWLQLEMFA
μ-CALPAIN fragment, Seq ID NO 58: LFKWLQLEMFA
μ-CALPAIN fragment, Seq ID NO 59: FKWLQLEMFA
μ-CALPAIN fragment, Seq ID NO 60: KWLQLEMFA
μ-CALPAIN fragment, Seq ID NO 61: WLQLEMFA
μ-CALPAIN fragment, Seq ID NO 62: LQLEMFA
μ-CALPAIN fragment, Seq ID NO 63: QLEMFA
μ-CALPAIN fragment, Seq ID NO 64: LEMFA
μ-CALPAIN fragment, Seq ID NO 65: EMFA
μ-CALPAIN fragment, Seq ID NO 66: DMFA
μ-CALPAIN fragment, Seq ID NO 67: LEMFA
μ-CALPAIN fragment, Seq ID NO 68: LDMFA
M-calpain Homo Sapiens, Seq ID NO 69:
MAGIAAKLAKDREAAEGLGSHERAIKYLNQDYEALRNECLEAGTLFQDPSFPAIPSALG
FKELGPYSSKTRGIEWKRPTEICADPQFIIGGATRTDICQGALGDCWLLAAIASLTLNEEI
LARVVPLNQSFQENYAGIFHFQFWQYGEWVEVVVDDRLPTKDGELLFVHSAEGSEFW
SALLEKAYAKINGCYEALSGGATTEGFEDFTGGIAEWYELKKPPPNLFKIIQKALQKGSL
LGCSIDITSAADSEAITFQKLVKGHAYSVTGAEEVESNGSLQKLIRIRNPWGEVEWTGR
WNDNCPSWNTIDPEERERLTRRHEDGEFWMSFSDFLRHYSRLEICNLTPDTLTSDTY
KKWKLTKMDGNWRRGSTAGGCRNYPNTFWMNPQYLIKLEEEDEDEEDGESGCTFLV
GLIQKHRRRQRKMGEDMHTIGFGIYEVPEELSGQTNIHLSKNFFLTNRARERSDTFINL
REVLNRFKLPPGEYILVPSTFEPNKDGDFCIRVFSEKKADYQAVDDEIEANLEEFDISED
DIDDGFRRLFAQLAGEDAEISAFELQTILRRVLAKRQDIKSDGFSIETCKIMVDMLDSDG
SGKLGLKEFYILWTKIQKYQKIYREIDVDRSGTMNSYEMRKALEEAGFKMPCQLHQVIV
ARFADDQLIIDFDNFVRCLVRLETLFKIFKQLDPENTGTIELDLISWLCFSVL
M-calpain, Mus Musculus. SEQ ID NO 70:
MAGIAIKLAKDREAAEGLGSHERAIKYLNQDYETLRNECLEAGALFQDPSFPALPSSLG
YKELGPYSSKTRGIEWKRPTEICADPQFIIGGATRTDICQGALGDCWLLAAIASLTLNEEI
LARVVPPDQSFQENYAGIFHFQFWQYGEWVEVVVDDRLPTKDGELLFVHSAEGSEF
WSALLEKAYAKINGCYETLSGGATTEGFEDFTGGIAEWYELRKPPPNLFKIIQKALEKG
SLLGCSIDITSAADSEAVTYQKLVKGHAYSVTGAEEVESSGSLQKLIRIRNPWGQVEWT
GKWNDNCPSWNTVDPEVRANLTERQEDGEFWMSFSDFLRHYSRLEICNLTPDTLTC
DSYKKWKLTKMDGNWRRGSTAGGCRNYPNTFWMNPQYLIKLEEEDEDEEDGGRGC
TFLVGLIQKHRRRQRKMGEDMHTIGFGIYEVPEELTGQTNIHLGKNFFLTTRARERSDT
FINLREVLNRFKLPPGEYVLVPSTFEPHKDGDFCIRVFSEKKADYQAVDDEIEANIEEID
ANEEDIDDGFRRLFVQLAGEDAEISAFELQTILRRVLAKRQDIKSDGFSIETCKIMVDML
DEDGSGKLGLKEFYILWTKIQKYQKIYREIDVDRSGTMNSYEMRKALEEAGFKLPCQLH
QVIVARFADDELIIDFDNFVRCLVRLETLFKIFKQLDPENTGTIQLNLASWLSFSVL
M-calpain Sus Scrofa, Seq ID NO 71:
MAGIAAKLAKDREAAEGLGSHERAVKYLNQDYAELRDQCLEAGALFQDPSFPALPSSL
GFKELGPYSGKTRGIEWKRPTEICDNPQFIIGGATRTDICQGALGDCWLLAAIASLTLNE
EVLARVVPLDQSFQENYAGIFRFQFWQYGEWVEVVVDDRLPTKDGELLFVHSAEGSE
FWSALLEKAYAKINGCYEALSGGATTEGFEDFTGGIAEWYELRKAPPNLFKIIQKALQK
GSLLGCSIDITSAADSEAVTFQKLVKGHAYSVTGAEEVESRGSLQKLIRIRNPWGEVEW
TGQWNDNCPNWNTVDPEVRESLTRRHEDGEFWMSFSDFLRHYSRLEICNLTPDTLTS
DSYKKWKLTKMDGNWRRGSTAGGCRNYPNTFWMNPQYLIKLEEEDEDQEDGESGC
TFLVGLIQKHRRRQRKMGEDMHTIGFGIYEVPEELTGQTNIHLSKNFFLTHRARERSDT
FINLREVLNRFKLPPGEYILVPSTFEPNKDGDFCIRVFSEKKADYQVVDDEIEADLEEND
ASEDDIDDGFRRLFAQLAGEDAEISAFELQTILRRVLAKRQDIKSDGFSIETCKIMVDML
DSDGSAKLGLKEFYILWTKIQKYQKIYREIDVDRSGTMNSYEMRKALEEAGFKLPCQLH
QVIVARFADDQLIIDFDNFVRCLVRLETLFRISKQLDSENTGTIELDLISWLCFSVL
M-calpain Rattus norvegicus Seq ID NO 72:
MAGIAMKLAKDREAAEGLGSHERAIKYLNQDYETLRNECLEAGALFQDPSFPALPSSL
GFKELGPYSSKTRGIEWKRPTEICADPQFIIGGATRTDICQGALGDCWLLAAIASLTLNE
EILARVVPLDQSFQENYAGIFHFQFWQYGEWVEVVVDDRLPTKDGELLFVHSAEGSEF
WSALLEKAYAKINGCYEALSGGATTEGFEDFTGGIAEWYELRKPPPNLFKIIQKALEKG
SLLGCSIDITSAADSEAVTYQKLVKGHAYSVTGAEEVESSGSLQKLIRIRNPWGQVEWT
GKWNDNCPSWNTVDPEVRANLTERQEDGEFWMSFSDFLRHYSRLEICNLTPDTLTC
DSYKKWKLTKMDGNWRRGSTAGGCRNYPNTFWMNPQYLIKLEEEDEDDEDGERGC
TFLVGLIQKHRRRQRKMGEDMHTIGFGIYEVPEELTGQTNIHLSKNFFLTTRARERSDT
FINLREVLNRFKLPPGEYVLVPSTFEPHKNGDFCIRVFSEKKADYQTVDDEIEANIEEIEA
NEEDIGDGFRRLFAQLAGEDAEISAFELQTILRRVLAKREDIKSDGFSIETCKIMVDMLD
EDGSGKLGLKEFYILWTKIQKYQKIYREIDVDRSGTMNSYEMRKALEEAGFKLPCQLH
QVIVARFADDELIIDFDNFVRCLVRLEILFKIFKQLDPENTGTIQLDLISWLSFSVL
M-calpain, Bos Taurus. Seq ID NO 73:
MAGIAAKLAKDREAAEGLGSHERAVKYLNQDYAALRDECLEAGALFQDPSFPALPSSL
GFKELGPYSSKTRGIEWKRPTEICDNPQFITGGATRTDICQGALGDCWLLAAIASLTLN
EEILARVVPLDQSFQENYAGIFHFQFWQYGEWVEVVVDDRLPTKDGELLFVHSAEGSE
FWSALLEKAYAKINGCYEALSGGATTEGFEDFTGGIAEWYELRKAPPNLFRIIQKALQK
GSLLGCSIDITSAADSEAITFQKLVKGHAYSVTGAEEVESRGSLQKLIRIRNPWGEVEW
TGQWNDNCPNWNTVDPEVRETLTRQHEDGEFWMSFNDFLRHYSRLEICNLTPDTLT
SDSYKKWKLTKMDGNWRRGSTAGGCRNYPNTFWMNPQYLIKLEEEDEDQEDGESG
CTFLVGLIQKHRRRQRKMGEDMHTIGFGIYEVPEELTGQTNIHLSKKFFLTTRARERSD
TFINLREVLNRFKLPPGEYIVVPSTFEPNKDGDFCIRVFSEKKADYQVVDDEIEANIDEID
ISEDDIDDGFRRLFAQLAGEDAEISAFELQTILRRVLAKRQDIKSDGFSIETCKIMVDMLD
SDGSGKLGLKEFYILWTKIQKYQKIYREIDVDRSGTMNSYEMRKALEEAGFKMPCQLH
QVIVARFADDDLIIDFDNFVRCLIRLETLFRIFKQLDPENTGMIQLDLISWLSFSVL
M-CALPAIN fragment, Seq ID NO 74: KQLDPENTGTIELDLISWLCFSVL
M-CALPAIN fragment, Seq ID NO 75: QLDPENTGTIELDLISWLCFSVL
M-CALPAIN fragment, Seq ID NO 76: LDPENTGTIELDLISWLCFSVL
M-CALPAIN fragment, Seq ID NO 77: DPENTGTIELDLISWLCFSVL
M-CALPAIN fragment, Seq ID NO 78: PENTGTIELDLISWLCFSVL
M-CALPAIN fragment, Seq ID NO 79: ENTGTIELDLISWLCFSVL
M-CALPAIN fragment, Seq ID NO 80: NTGTIELDLISWLCFSVL
M-CALPAIN fragment, Seq ID NO 81: TGTIELDLISWLCFSVL
M-CALPAIN fragment, Seq ID NO 82: GTIELDLISWLCFSVL
M-CALPAIN fragment, Seq ID NO 83: TIELDLISWLCFSVL
M-CALPAIN fragment, Seq ID NO 84: IELDLISWLCFSVL
M-CALPAIN fragment, Seq ID NO 85: IELDLISWLCFDVL
M-CALPAIN fragment, Seq ID NO 86: IELDLISWLCFEVL
M-CALPAIN fragment, Seq ID NO 87: ELDLISWLCFSVL
M-CALPAIN fragment, Seq ID NO 88: LDLISWLCFSVL
M-CALPAIN fragment, Seq ID NO 89: DLISWLCFSVL
M-CALPAIN fragment, Seq ID NO 90: LDLISWLCFDVL
M-CALPAIN fragment, Seq ID NO 91: LDLISWLCFEVL
M-CALPAIN fragment, Seq ID NO 92: LISWLCFSVL
M-CALPAIN fragment, Seq ID NO 93: ISWLCFSVL
M-CALPAIN fragment, Seq ID NO 94: KQLDPENTGTIELDLISWLCFDVL
M-CALPAIN fragment, Seq ID NO 95: QLDPENTGTIELDLISWLCFDVL
M-CALPAIN fragment, Seq ID NO 96: LDPENTGTIELDLISWLCFDVL
M-CALPAIN fragment, Seq ID NO 97: DPENTGTIELDLISWLCFDVL
M-CALPAIN fragment, Seq ID NO 98: PENTGTIELDLISWLCFDVL
M-CALPAIN fragment, Seq ID NO 99: ENTGTIELDLISWLCFDVL
M-CALPAIN fragment, Seq ID NO 100: NTGTIELDLISWLCFDVL
M-CALPAIN fragment, Seq ID NO 101: TGTIELDLISWLCFDVL
M-CALPAIN fragment, Seq ID NO 102: GTIELDLISWLCFDVL
M-CALPAIN fragment, Seq ID NO 103: TIELDLISWLCFDVL
M-CALPAIN fragment, Seq ID NO 104: IELDLISWLCFDVL
M-CALPAIN fragment, Seq ID NO 105: IELDLISWLCFDVL
M-CALPAIN fragment, Seq ID NO 106: ELDLISWLCFDVL
M-CALPAIN fragment, Seq ID NO 107: LDLISWLCFDVL
M-CALPAIN fragment, Seq ID NO 108: DLISWLCFDVL
M-CALPAIN fragment, Seq ID NO 109: KQLDPENTGTIELDLISWLCFEVL
M-CALPAIN fragment, Seq ID NO 110: QLDPENTGTIELDLISWLCFEVL
M-CALPAIN fragment, Seq ID NO 111: LDPENTGTIELDLISWLCFEVL
M-CALPAIN fragment, Seq ID NO 112: DPENTGTIELDLISWLCFEVL
M-CALPAIN fragment, Seq ID NO 113: PENTGTIELDLISWLCFEVL
M-CALPAIN fragment, Seq ID NO 114: ENTGTIELDLISWLCFEVL
M-CALPAIN fragment, Seq ID NO 115: NTGTIELDLISWLCFEVL
M-CALPAIN fragment, Seq ID NO 116: TGTIELDLISWLCFDVL
M-CALPAIN fragment, Seq ID NO 117: GTIELDLISWLCFEVL
M-CALPAIN fragment, Seq ID NO 118: TIELDLISWLCFEVL
M-CALPAIN fragment, Seq ID NO 119: IELDLISWLCFEVL
M-CALPAIN fragment, Seq ID NO 120: IELDLISWLCFEVL
M-CALPAIN fragment, Seq ID NO 121: ELDLISWLCFDVL
M-CALPAIN fragment, Seq ID NO 122: LDLISWLCFDVL
M-CALPAIN fragment, Seq ID NO 123: DLISWLCFDVL
M-CALPAIN fragment, Seq ID NO 124: LISWLCFSVL
M-CALPAIN fragment, Seq ID NO 125: ISWLCFSVL
M-CALPAIN fragment, Seq ID NO 126: ISWLCFDVL
M-CALPAIN fragment, Seq ID NO 127: ISWLCFEVL
M-CALPAIN fragment, Seq ID NO 128: SWLCFSVL
M-CALPAIN fragment, Seq ID NO 129: SWLCFDVL
M-CALPAIN fragment, Seq ID NO 130: SWLCFEVL
M-CALPAIN fragment, Seq ID NO 131: WLCFSVL
M-CALPAIN fragment, Seq ID NO 132: ISWLCFDVL
M-CALPAIN fragment, Seq ID NO 133: ISWLCFEVL
M-CALPAIN fragment, Seq ID NO 134: SWLCFSVL
M-CALPAIN fragment, Seq ID NO 135: SWLCFDVL
M-CALPAIN fragment, Seq ID NO 136: SWLCFEVL
M-CALPAIN fragment, Seq ID NO 137: WLCFSVL
M-CALPAIN fragment, Seq ID NO 138: LCFDVL
M-CALPAIN fragment, Seq ID NO 139: LCFEVL
M-CALPAIN fragment, Seq ID NO 140: CFSVL
M-CALPAIN fragment, Seq ID NO 141: CFDVL
M-CALPAIN fragment, Seq ID NO 142: CFEVL
M-CALPAIN fragment, Seq ID NO 143: FSVL
M-CALPAIN fragment, Seq ID NO 144: FDVL
M-CALPAIN fragment, Seq ID NO 145: FEVL
CONNECTION BETWEEN PHOSPHATASE DOMAIN AND LIPID-BINDING DOMAIN:
PTEN fragment, SEQ ID NO 146: IPSQRRYVYYYSYLLKNHLDYRPV
UNDERLINED IS ALPHA-HELIX; BLUE IS EXPOSED LINKER; RED IS M-CALPAIN CLEAVAGE SITE.
PTEN fragment, SEQ ID NO 147: PSQRRYVYYYSYLLKNHLDYRP
PTEN fragment, SEQ ID NO 148: PSQRRYVYYYSYLLKNHLDYRP
PTEN fragment, SEQ ID NO 149: PSQRRYVYYYSYLLKNHLDYR
PTEN fragment, SEQ ID NO 150: PSQRRYVYYYSYLLKNHLDY
PTEN fragment, SEQ ID NO 151: SQRRYVYYYSYLLKNHLD
PTEN fragment, SEQ ID NO 152: PSQRRYVYYYSYLLKNHLD
PTEN fragment, SEQ ID NO 153: PSQRRYVYYYSYLLKNHL
PTEN fragment, SEQ ID NO 154: PSQRRYVYYYSYLLKNH
PTEN fragment, SEQ ID NO 155: PSQRRYVYYYSYLLKN
PTEN fragment, SEQ ID NO 156: PSQRRYVYYYSYLLK
PTEN fragment, SEQ ID NO 157: SQRRYVYYYSYLLKNHL
PTEN fragment, SEQ ID NO 158: QRRYVYYYSYLLKNHL
PTEN fragment, SEQ ID NO 159: QRRYVYYYSYLLKNH
PTEN fragment, SEQ ID NO 160: QRRYVYYYSYLLKN
PTEN fragment, SEQ ID NO 161: QRRYVYYYSYLLK
PTEN fragment, SEQ ID NO 162: RRYVYYYSYLLK
PTEN fragment, SEQ ID NO 163: QRRYVYYYSYLLKNHLDY
PTEN fragment, SEQ ID NO 164: RRYVYYYSYLLKNHLDY
PTEN fragment, SEQ ID NO 165: YVYYYSYLLKNHLDY
PTEN fragment, SEQ ID NO 166: YYYSYLLKNHLDY
PTEN fragment, SEQ ID NO 167: YYSYLLKNHLDY
PTEN fragment, SEQ ID NO 168: YSYLLKNHLDY
PTEN fragment, SEQ ID NO 169: SYLLKNHLDY
PTEN fragment, SEQ ID NO 170: YLLKNHLDY
PTEN fragment, SEQ ID NO 171: LLKNHLDY
PTEN fragment, SEQ ID NO 172: YYSYLLKNHLD
PTEN fragment, SEQ ID NO 173: YSYLLKNHL
PTEN fragment, SEQ ID NO 174: SYLLKNH
PTEN fragment, SEQ ID NO 175: YLLKN
PTEN fragment, SEQ ID NO 176: YLLKNHLD
PTEN fragment, SEQ ID NO 177: YLLK
PTEN fragment, SEQ ID NO 178: LLKN
PTEN fragment, SEQ ID NO 179:
ERADNDKEYLVLTLTKNDLDKANKDKANRYFSPNFKVKLYFTKTVEEPSNPE
UNDERLINED IS LIPID-BINDING DOMAIN; RED ARE M-CALPAIN CLEAVAGE SITES.
PTEN fragment, SEQ ID NO 180: NRYFSPNFKVKLYFTKTVEEPSNPE
PTEN fragment, SEQ ID NO 181:
KEYLVLTLTKNDLDKANKDKANRYFSPNFKVKLYFTKTVEE
PTEN fragment, SEQ ID NO 182: ERADNDKEYLVLTLTKNDLDKANKD
PTEN fragment, SEQ ID NO 183: KEYLVLTLTKND
PTEN fragment, SEQ ID NO 184: EYLVLTLTKN
PTEN fragment, SEQ ID NO 185: EYLVLTLTK
PTEN fragment, SEQ ID NO 186: KEYLVLTLTK
PTEN fragment, SEQ ID NO 187: YLVLTLTK
PTEN fragment, SEQ ID NO 188: LVLTLT
PTEN fragment, SEQ ID NO 189: VLTLT
PTEN fragment, SEQ ID NO 190: VLTL
PTEN fragment, SEQ ID NO 191: LTLT
PTEN fragment, SEQ ID NO 192: EYLVL
PTEN fragment, SEQ ID NO 193: EYLV
Membrane transduction domains
7-mer, SEQ ID NO 194: -RRMKWKK-
Transportan SEQ ID NO 195: -GWTLNSAGYLLGKINLKALAALAKISIL-amide
PENATRIN, SEQ ID NO 196: -RQIKIWFQNRRMKWKK-
PolyArginine, SEQ ID NO 197: -RRRRRRRRRR-
MAP, SEQ ID NO 198: -LLIILRRRIRKQAHAHSK-
RDP, SEQ ID NO 199: -KSVRTWNEIIPSKGCLRVGGRCHPHVNGGGRRRRRRRRR-
HIV-TAT SEQ ID NO 200: -RKKRRQRRR
More PTEN Derived m-Calpain selective peptides
SEQ ID NO 201: NRYFSPNFKVKLYFTKTVEEPSNP
SEQ ID NO 202: RYFSPNFKVKLYFTKTVEEPSNP
SEQ ID NO 203: RYFSPNFKVKLYFTKTVEEPSN
SEQ ID NO 204: YFSPNFKVKLYFTKTVEEPSN
SEQ ID NO 205: YFSPNFKVKLYFTKTVEEPS
SEQ ID NO 206: FSPNFKVKLYFTKTVEEPS
SEQ ID NO 207: FSPNFKVKLYFTKTVEEP
SEQ ID NO 208: SPNFKVKLYFTKTVEEP
SEQ ID NO 209: SPNFKVKLYFTKTVEE
SEQ ID NO 210: PNFKVKLYFTKTVEE
SEQ ID NO 211: PNFKVKLYFTKTVE
SEQ ID NO 212: NFKVKLYFTKTVE
SEQ ID NO 213: NFKVKLYFTKTV
SEQ ID NO 214: FKVKLYFTKTV
SEQ ID NO 215: FKVKLYFTKT
SEQ ID NO 216: KVKLYFTKT
SEQ ID NO 217: KVKLYFTK
SEQ ID NO 218: VKLYFTK
SEQ ID NO 219: VKLYFT
SEQ ID NO 220: KLYFT
SEQ ID NO 221: KLYF
SEQ ID NO 222: LYF

1. A composition comprising a pharmaceutically acceptable excipient and a molecule of formula: 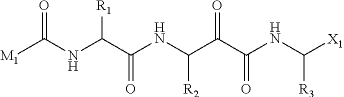
wherein:
M₁ is —O, —N, —S, or —C substituted to covalently link a blocking group selected from Y₁—PhCH₂—, Y₁—Ph(CH₂)₂—, PhCH₂—Y₁, or Ph(CH₂)₂—Y₁—,
wherein Y₁ is a polypeptide, or modified polypeptide covalently linked for improving half-life, bioavailability or targeting; or wherein
Y₁ is —H, a substitution for linking small molecule, polypeptide, or modified polypeptide moieties for improving half-life, bioavailability or targeting; or
Y₁ is an —O, —N, —S, or —C substitution to link polypeptides that improve membrane permeability or blood brain barrier passage selected from (SEQ ID NOs: 194-200), a transferrin polypeptide fragment, an insulin fragment, an LDL binding protein fragment, a rabies virus glycoprotein fragment,
or M₁ is —O, —N, —S, or —C substituted to covalently link a small molecule, polypeptide, or modified polypeptide that improves membrane permeability or blood brain barrier passage, a polypeptide selected from SEQ ID NOs: (194-200), a transferrin polypeptide fragment, an insulin fragment, an LDL binding protein fragment, a rabies virus glycoprotein fragment;
R₁ is a functional group covalently bonded to the alpha-carbon having an L orientation, and having an amino acid side chain of leucine, phenylalanine, tyrosine, valine, isoleucine, methionine, alanine, or a modified amino acid side chain; and
R₂ is —CH₃, —CH₂CH₃, —(CH₂)₂CH₃, —CH(CH₃)₂, —CH₂CH(CH₃)₂, —CH(CH₃)CH₂CH₃, —C₆H₅, —C₆H₄(4-OH), C₆H₄(3-OH), C₆H₄(2-OH), C₆H₄(2-CH₃), C₆H₄(3-CH₃), C₆H₄(4-CH₃), C₆H₄(2-OCH₃), C₆H₄(3-OCH₃), C₆H₄(4-OCH₃), C₆H₄(2-NH₂), C₆H₄(3-NH₂), C₆H₄(4-NH₂), C₆H₄(2- NHCH₃), C₆H₄(3-NHCH₃), C₆H₄(4-NHCH₃), C₆H₄(2-N(CH₃)₂), C₆H₄(3-N(CH₃)₂), or C₆H₄(4-N(CH₃)₂);
R₃ is —H, —OCH₃, ═NH, —NH₂, —SH, ═O, ═S, —OCH₂CH₃, —O(CH₂)₂CH₃, —OCH(CH₃)₂, —SCH₃, —SCH₂CH₃, —S(CH₂)₂CH₃, —SCH(CH₃)₂, —OH, —CH₃, CH₂CH₃, —F, —Cl, —Br, —I;
X₁ is —C₆H₃(3,5-R₄,R₅), —CHR₆—C₆H₃-(3,5-R₄,R₅), -2-pyridyl, -2-pyridyl(3,5, R₄,R₅), —CHR₆-2-pyridyl(3,5, R₄,R₅), -3-pyridyl(3,5, R₄, R₅), —CHR₆-3-pyridyl(3,5,R₄,R₅), -4-pyridyl(3,5, R₄, R₅), or —CHR₆-4-pyridyl(3,5,R₄,R₅); wherein
R₄ is —H, —OCH₃, —OCH₂CH₃, —O(CH₂)₂CH₃, —OCH(CH₃)₂, —SCH₃, SCH₂CH₃, S(CH₂)₂CH₃, —SCH(CH₃)₂, —OH, —CH₃, —CH₂CH₃, —CN, —CHNH, —NH₂, —NHCH₃, —N(CH₃)₂, —F, —Cl, —Br, or —I;
R₅ is —H, —OCH₃, —OCH₂CH₃, —O(CH₂)₂CH₃,—OCH(CH₃)₂, —SCH₃, SCH₂CH₃, —S(CH₂)₂CH₃, —SCH(CH₃)₂, —OH, —CH₃, —CH₂CH₃, —CN, —CHNH, —NH₂, —NHCH₃, —N(CH₃)₂, —F, —Cl, —Br, or —I; and
R₆ is —H, —OCH₃, —OCH₂CH₃, —O(CH₂)₂CH₃, —OCH(CH₃)₂, —SCH₃, —SCH₂CH₃, —S(CH₂)₂CH₃, —SCH(CH₃)₂, —OH, —CH₃, —CH₂CH₃, —CN, —CHNH, —NH₂, —NHCH₃, —N(CH₃)₂, —F, —Cl, —Br, or —I.

2. A composition comprising a pharmaceutically acceptable excipient and a molecule of formula: 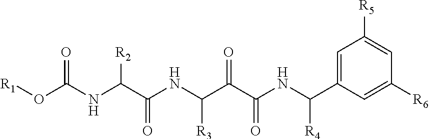
wherein:
R₁ is X₁—PhCH₂—, or X₁—Ph(CH₂)₂—; wherein
X₁ is —H, or a substitution for linking a small molecule, polypeptide, modified polypeptide moiety, wherein the small molecule, polypeptide, modified polypeptide moiety improves half-life, bioavailability or targeting;
R₂ is a functional group covalently bonded to the alpha-carbon, having an L orientation, and having an amino acid side chain of leucine, phenylalanine, tyrosine, valine, isoleucine, methionine, alanine, or a modified amino acid side chain;
R₃ is —CH₃, —CH₂CH₃, —(CH₂)₂CH₃, —CH(CH₃)₂—CH₂CH(CH₃)₂, —CH(CH₃)CH₂CH₃, —C₆H₅, —C₆H₄(4-OH), C₆H₄(3-OH), C₆H₄(2-OH), C₆H₄(2-CH₃), C₆H₄(3-CH₃), C₆H₄(4-CH₃), C₆H₄(2-OCH₃), C₆H₄(3-OCH₃), C₆H₄(4-OCH₃), C₆H₄(2-NH₂), C₆H₄(3-NH₂), C₆H₄(4-NH₂), C₆H₄(2- NHCH₃), C₆H₄(3-NHCH₃), C₆H₄(4-NHCH₃), C₆H₄(2-N(CH₃)₂), C₆H₄(3-N(CH₃)₂), or C₆H₄(4-N(CH₃)₂);
R₄ is —H, or —OCH₃, ═NH, —NH₂, —SH, ═O, ═S, —OCH₂CH₃, —O(CH₂)₂CH₃, —OCH(CH₃)₂, —SCH₃, SCH₂CH₃, —S(CH₂)₂CH₃, —SCH(CH₃)₂, —OH, —CH₃, —CH₂CH₃, —F, —Cl, —Br, or —I;
R₅ is —H, —OCH₃, —OCH₂CH₃, —O(CH₂)₂CH₃, —OCH(CH₃)₂, —SCH₃, SCH₂CH₃, —S(CH₂)₂CH₃, —SCH(CH₃)₂, —OH, —CH₃, —CH₂CH₃, —CN, —CHNH, —NH₂, —NHCH₃, —N(CH₃)₂, —F, —Cl, —Br, —I; and
R₆ is —H, —OCH₃, —OCH₂CH₃, —O(CH₂)₂CH₃, —OCH(CH₃)₂, —SCH₃, SCH₂CH₃, —S(CH₂)₂CH₃, —SCH(CH₃)₂, —OH, —CH₃, —CH₂CH₃, —CN, —CHNH, —NH₂, —NHCH₃, —N(CH₃)₂, —F, —Cl, —Br, or —I.

3. A composition comprising a pharmaceutically acceptable excipient and a molecule of formula: 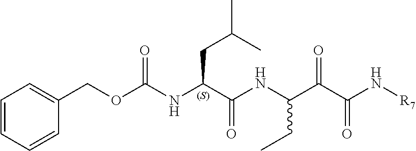
wherein R₇ is 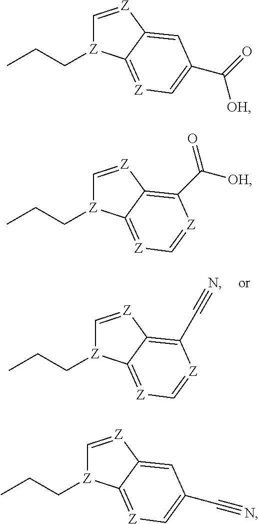
wherein
Z is a CH or a N.

4. A composition comprising a pharmaceutically acceptable excipient and a molecule of formula:
wherein R₈ is

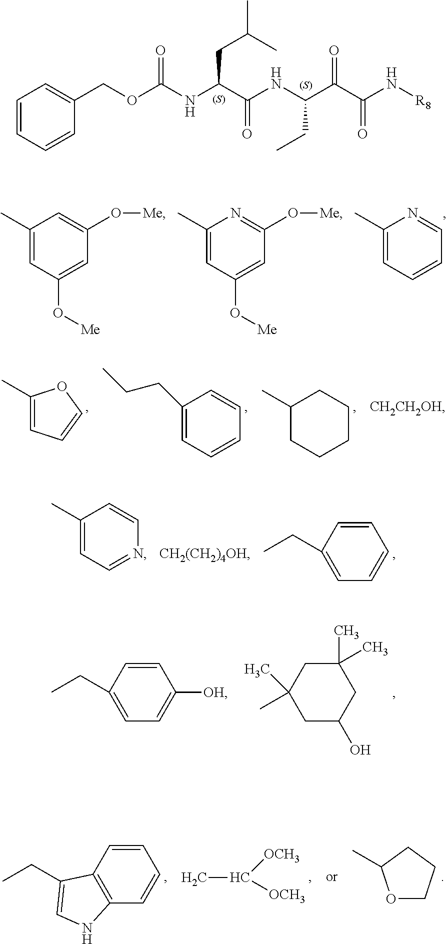

5. A composition comprising a molecule according to claim 1, wherein its calpain-2 inhibition constant (Ki) is equal to, or more, than 10-fold lower than its Ki for calpain-1.

6. A composition according to claim 5, wherein the molecule inhibits neuronal cell death, enhances memory.

7. (canceled)

8. A method of treating glaucoma or a neurological disease, comprising administering a composition according to claim 5.

9. (canceled)

10. A composition comprising a pharmaceutically acceptable excipient and a synthetic polypeptide having at least 95% identity to the entirety of any one of SEQ ID NO: 1-68, 74-193, or 201-222, wherein the synthetic polypeptide has a calpain-2 inhibition constant (Ki) that is equal to, or more, than 10-fold lower than its Ki for calpain-1.

11. A composition according to claim 10, wherein the synthetic polypeptide additionally comprises a membrane transduction synthetic polypeptide.

12-15. (canceled)

16. A composition according to claim 10, wherein the molecule inhibits neuronal cell death, or enhances memory.

17. (canceled)

18. A method of treating glaucoma, or treating a neurological disease, comprising administering a composition according to claim 10.

19. (canceled)

20. A composition according to claim 11, wherein the molecule inhibits neuronal cell death, or enhances memory.

21. A method of treating glaucoma, a neurological disease, comprising administering a composition according to claim 11.

22. A composition comprising a molecule according to claim 2, wherein its calpain-2 inhibition constant (Ki) is equal to, or more, than 10-fold lower than its Ki for calpain-1.

23. A composition comprising a molecule according to claim 3, wherein its calpain-2 inhibition constant (Ki) is equal to, or more, than 10-fold lower than its Ki for calpain-1.

24. A composition comprising a molecule according to claim 4, wherein its calpain-2 inhibition constant (Ki) is equal to, or more, than 10-fold lower than its Ki for calpain-1.

25. A composition according to claim 22, wherein the molecule inhibits neuronal cell death, enhances memory.

26. A method of treating glaucoma, or a neurological disease, comprising administering a composition according to claim 22.

27. A composition according to claim 23, wherein the molecule inhibits neuronal cell death, enhances memory.

28. A method of treating glaucoma, or a neurological disease, comprising administering a composition according to claim 23.

29. A composition according to claim 24, wherein the molecule inhibits neuronal cell death, enhances memory.

30. A method of treating glaucoma, or a neurological disease, comprising administering a composition according to claim 24.


SEQUENCE LISTING (221 sequences)

SEQ ID NO: 1 - protein, 403 aa, Homo sapiens [sequence omitted]

SEQ ID NO: 2 - protein, 714 aa, Homo sapiens
MSEEIITPVYCTGVSAQVQKQRARELGLGRHENAIKYLGQDYEQLRVRCLQSGTLFRDEAFPPVPQSLGYKDLGPNSSKTYGIKWKRPTELLSNPQFIVDGATRTDICQGALGDCWLLAAIASLTLNDTLLHRVVPHGQSFQNGYAGIFHFQLWQFGEWVDVVVDDLLPIKDGKLVFVHSAEGNEFWSALLEKAYAKVNGSYEALSGGSTSEGFEDFTGGVTEWYELRKAPSDLYQIILKALERGSLLGCSIDISSVLDMEAITFKKLVKGHAYSVTGAKQVNYRGQVVSLIRMRNPWGEVEWTGAWSDSSSEWNNVDPYERDQLRVKMEDGEFWMSFRDFMREFTRLEICNLTPDALKSRTIRKWNTTLYEGTWRRGSTAGGCRNYPATFWVNPQFKIRLDETDDPDDYGDRESGCSFVLALMQKHRRRERRFGRDMETIGFAVYEVPPELVGQPAVHLKRDFFLANASRARSEQFINLREVSTRFRLPPGEYVVVPSTFEPNKEGDFVLRFFSEKSAGTVELDDQIQANLPDEQVLSEEEIDENFKALFRQLAGEDMEISVKELRTILNRIISKHKDLRTKGFSLESCRSMVNLMDRDGNGKLGLVEFNILWNRIRNYLSIFRKFDLDKSGSMSAYEMRMAIESAGFKLNKKLYELIITRYSEPDLAVDFDNFVCCLVRLETMFRFFKTLDTDLDGVVTFDLFKWLQLTMFA

SEQ ID NO: 3 - protein, 713 aa, Mus musculus
MTEELITPVYCTGVSAQVQKKRDKELGLGRHENAIKYLGQDYETLRARCLQSGVLFQDEAFPPVSHSLGFKELGPHSSKTYGIKWKRPTELMSNPQFIVDGATRTDICQGALGDCWLLAAIASLTLNETILHRVVPYGQSFQDGYAGIFHFQLWQFGEWVDVVIDDLLPTKDGKLVFVHSAQGNEFWSALLEKAYAKVNGSYEALSGGCTSEAFEDFTGGVTEWYDLQKAPSDLYQIILKALERGSLLGCSINISDIRDLEAITFKNLVRGHAYSVTGAKQVTYQGQRVNLIRMRNPWGEVEWKGPWSDSSYEWNKVDPYEREQLRVKMEDGEFWMSFRDFIREFTKLEICNLTPDALKSRTLRNWNTTFYEGTWRRGSTAGGCRNYPATFWVNPQFKIRLEEVDDADDYDNRESGCSFLLALMQKHRRRERRFGRDMETIGFAVYQVPRELAGQPVHLKRDFFLANASRAQSEHFINLREVSNRIRPPPGEYIVVPSTFEPNKEGDFLLRFFSEKKAGTQELDDQIQANLPDEKVLSEEEIDDNFKTLFSKLAGDDMEISVKELQTILNRIISKHKDLRTNGFSLESCRSMVNLMDRDGNGKLGLVEFNILWNRIRNYLTIFRKFDLDKSGSMSAYEMRMAIEAAGFKLNKKLHELIITRYSEPDLAVDFDNFVCCLVRLETMFRFFKLLDTDLNGVVTFDLFKWLQLTMFA

SEQ ID NO: 4 - protein, 716 aa, Bos taurus
MAEEFITPVYCTGVSAQVQKQRAKELGLGRHENAIKYLGQDYEQLRVHCLQRGALFRDEAFPPVPQSLGFKELGPNSSKTYGIKWKRPTELFSNPQFIVDGATRTDICQGALGDCWLLAAIASLTLNDTLLHRVVPHGQSFQDGYAGIFHFQLWQFGEWVDVVVDDLLPTKDGKLVFVHSAQGNEFWSALLEKAYAKVNGSYEALSGGSTSEGFEDFTGGVTEWYELRKAPSDLYNIILKALERGSLLGCSIDISSILDMEAVTFKKLVKGHAYSVTGAKQVNYQGQMVNLIRMRNPWGEVEWTGAWSDGSSEWNGVDPYMREQLRVKMEDGEFWMSFRDFMREFTRLEICNLTPDALKSQRFRNWNTTLYEGTWRRGSTAGGCRNYPATFWVNPQFKIRLEETDDPDPDDYGGRESGCSFLLALMQKHRRRERRFGRDMETIGFAVYEVPPELMGQPAVHLKRDFFLSNASRARSEQFINLREVSTRFRLPPGEYVVVPSTFEPNKEGDFVLRFFSEKSAGTQELDDQVQANLPDEQVLSEEEIDENFKSLFRQLAGEDMEISVKELRTILNRIISKHKDLRTTGFSLESCRSMVNLMDRDGNGKLGLVEFNILWNRIRNYLSIFRKFDLDKSGSMSAYEMRMAIEFAGFKLNKKLYELIITRYSEPDLAVDFDNFVCCLVRLETMFRFFKTLDTDLDGVVTFDLFKWLQLTMFA

SEQ ID NO: 5 - protein, 713 aa, Rattus norvegicus
MAEELITPVYCTGVSAQVQKQRDKELGLGRHENAIKYLGQDYENLRARCLQNGVLFQDDAFPPVSHSLGFKELGPNSSKTYGIKWKRPTELLSNPQFIVDGATRTDICQGALGDCWLLAAIASLTLNETILHRVVPYGQSFQEGYAGIFHFQLWQFGEWVDVVVDDLLPTKDGKLVFVHSAQGNEFWSALLEKAYAKVNGSYEALSGGCTSEAFEDFTGGVTEWYDLQKAPSDLYQIILKALERGSLLGCSINISDIRDLEAITFKNLVRGHAYSVTDAKQVTYQGQRVNLIRMRNPWGEVEWKGPWSDNSYEWNKVDPYEREQLRVKMEDGEFWMSFRDFIREFTKLEICNLTPDALKSRTLRNWNTTFYEGTWRRGSTAGGCRNYPATFWVNPQFKIRLEEVDDADDYDSRESGCSFLLALMQKHRRRERRFGRDMETIGFAVYQVPRELAGQPVHLKRDFFLANASRAQSEHFINLREVSNRIRLPPGEYIVVPSTFEPNKEGDFLLRFFSEKKAGTQELDDQIQANLPDEKVLSEEEIDDNFKTLFSKLAGDDMEISVKELQTILNRIISKHKDLRTNGFSLESCRSMVNLMDRDGNGKLGLVEFNILWNRIRNYLTIFRKFDLDKSGSMSAYEMRMAIEAAGFKLNKKLHELIITRYSEPDLAVDFDNFVCCLVRLETMFRFFKILDTDLDGVVTFDLFKWLQLTMFA

SEQ ID NO: 6 - protein, 714 aa, Sus scrofa
MAEEVITPVYCTGVSAQVQKLRAKELGLGRHENAIKYLGQDYEQLRAHCLQSGSLFRDEAFPPVPQSLGFKELGPNSSKTYGVKWKRPTELFSNPQFIVDGATRTDICQGALGDCWLLAAIASLTLNDTLLHRVVPHGQSFQNGYAGIFHFQLWQFGEWVDVVVDDLLPTKDGKLVFVHSAQGNEFWSALLEKAYAKVNGSYEALSGGSTSEGFEDFTGGVTEWYELRKAPSDLYSIILKALERGSLLGCSIDISSVLDMEAVTFKKLVKGHAYSVTGAKQVNYQGQMVNLIRMRNPWGEVEWTGAWSDGSSEWNGVDPYQRDQLRVRMEDGEFWMSFRDFLREFTRLEICNLTPDALKSQRVRNWNTTLYEGTWRRGSTAGGCRNYPATFWVNPQFKIRLEETDDPEDDYGGRESGCSFVLALMQKHRRRERRFGRDMETIGFAVYEVPPELVGQPVHLKRDFFLANASRARSEQFINLREVSTRFRLPPGEYVVVPSTFEPNKEGDFVLRFFSEKKAGTQELDDQVQAILPDEQVLSEEEIDENFKALFRQLAGEDMEISVRELRTILNRIISKHKDLRTKGFSLESCRSMVNLMDRDGNGKLGLVEFNILWNRIRNYLSIFRKFDLDKSGSMSAYEMRMAIESAGFKLNKKLFELIITRYSEPDLAVDFDNFVCCLVRLETMFRFFKTLDTDLDGVVTFDLFKWLQLTMFA

SEQ ID NO: 7 - protein, 23 aa, Homo sapiens
LDTDLDGVVTFDLFKWLQLTMFA

SEQ ID NO: 8 - protein, 22 aa, Homo sapiens
DTDLDGVVTFDLFKWLQLTMFA

SEQ ID NO: 9 - protein, 21 aa, Homo sapiens
TDLDGVVTFDLFKWLQLTMFA

SEQ ID NO: 10 - protein, 20 aa, Homo sapiens
DLDGVVTFDLFKWLQLTMFA

SEQ ID NO: 11 - protein, 19 aa, Homo sapiens
LDGVVTFDLFKWLQLTMFA

SEQ ID NO: 12 - protein, 18 aa, Homo sapiens
DGVVTFDLFKWLQLTMFA

SEQ ID NO: 13 - protein, 17 aa, Homo sapiens
GVVTFDLFKWLQLTMFA

SEQ ID NO: 14 - protein, 16 aa, Homo sapiens
VVTFDLFKWLQLTMFA

SEQ ID NO: 15 - protein, 15 aa, Homo sapiens
VTFDLFKWLQLTMFA

SEQ ID NO: 16 - protein, 14 aa, Homo sapiens
TFDLFKWLQLTMFA

SEQ ID NO: 17 - protein, 13 aa, Homo sapiens
FDLFKWLQLTMFA

SEQ ID NO: 18 - protein, 12 aa, Homo sapiens
DLFKWLQLTMFA

SEQ ID NO: 19 - protein, 11 aa, Homo sapiens
LFKWLQLTMFA

SEQ ID NO: 20 - protein, 10 aa, Homo sapiens
FKWLQLTMFA

SEQ ID NO: 21 - protein, 9 aa, Homo sapiens
KWLQLTMFA

SEQ ID NO: 22 - protein, 8 aa, Homo sapiens
WLQLTMFA

SEQ ID NO: 23 - protein, 7 aa, Homo sapiens
LQLTMFA

SEQ ID NO: 24 - protein, 6 aa, Homo sapiens
QLTMFA

SEQ ID NO: 25 - protein, 5 aa, Homo sapiens
LTMFA

SEQ ID NO: 26 - protein, 4 aa, Homo sapiens
TMFA

SEQ ID NO: 27 - protein, 23 aa, Homo sapiens
LDTDLDGVVTFDLFKWLQLDMFA

SEQ ID NO: 28 - protein, 22 aa, Homo sapiens
DTDLDGVVTFDLFKWLQLDMFA

SEQ ID NO: 29 - protein, 21 aa, Homo sapiens
TDLDGVVTFDLFKWLQLDMFA

SEQ ID NO: 30 - protein, 20 aa, Homo sapiens
DLDGVVTFDLFKWLQLDMFA

SEQ ID NO: 31 - protein, 19 aa, Homo sapiens
LDGVVTFDLFKWLQLDMFA

SEQ ID NO: 32 - protein, 18 aa, Homo sapiens
DGVVTFDLFKWLQLDMFA

SEQ ID NO: 33 - protein, 17 aa, Homo sapiens
GVVTFDLFKWLQLDMFA

SEQ ID NO: 34 - protein, 16 aa, Homo sapiens
VVTFDLFKWLQLDMFA

SEQ ID NO: 35 - protein, 15 aa, Homo sapiens
VTFDLFKWLQLDMFA

SEQ ID NO: 36 - protein, 14 aa, Homo sapiens
TFDLFKWLQLDMFA

SEQ ID NO: 37 - protein, 13 aa, Homo sapiens
FDLFKWLQLDMFA

SEQ ID NO: 38 - protein, 12 aa, Homo sapiens
DLFKWLQLDMFA

SEQ ID NO: 39 - protein, 11 aa, Homo sapiens
LFKWLQLDMFA

SEQ ID NO: 40 - protein, 10 aa, Homo sapiens
FKWLQLDMFA

SEQ ID NO: 41 - protein, 9 aa, Homo sapiens
KWLQLDMFA

SEQ ID NO: 42 - protein, 8 aa, Homo sapiens
WLQLDMFA

SEQ ID NO: 43 - protein, 7 aa, Homo sapiens
LQLDMFA

SEQ ID NO: 44 - protein, 6 aa, Homo sapiens
QLDMFA

SEQ ID NO: 45 - protein, 5 aa, Homo sapiens
LDMFA

SEQ ID NO: 46 - protein, 23 aa, Homo sapiens
LDTDLDGVVTFDLFKWLQLEMFA

SEQ ID NO: 47 - protein, 22 aa, Homo sapiens
DTDLDGVVTFDLFKWLQLEMFA

SEQ ID NO: 48 - protein, 21 aa, Homo sapiens
TDLDGVVTFDLFKWLQLEMFA

SEQ ID NO: 49 - protein, 20 aa, Homo sapiens
DLDGVVTFDLFKWLQLEMFA

SEQ ID NO: 50 - protein, 19 aa, Homo sapiens
LDGVVTFDLFKWLQLEMFA

SEQ ID NO: 51 - protein, 18 aa, Homo sapiens
DGVVTFDLFKWLQLEMFA

SEQ ID NO: 52 - protein, 17 aa, Homo sapiens
GVVTFDLFKWLQLEMFA

SEQ ID NO: 53 - protein, 16 aa, Homo sapiens
VVTFDLFKWLQLEMFA

SEQ ID NO: 54 - protein, 15 aa, Homo sapiens
VTFDLFKWLQLEMFA

SEQ ID NO: 55 - protein, 14 aa, Homo sapiens
TFDLFKWLQLEMFA

SEQ ID NO: 56 - protein, 13 aa, Homo sapiens
FDLFKWLQLEMFA

SEQ ID NO: 57 - protein, 12 aa, Homo sapiens
DLFKWLQLEMFA

SEQ ID NO: 58 - protein, 11 aa, Homo sapiens
LFKWLQLEMFA

SEQ ID NO: 59 - protein, 10 aa, Homo sapiens
FKWLQLEMFA

SEQ ID NO: 60 - protein, 9 aa, Homo sapiens
KWLQLEMFA

SEQ ID NO: 61 - protein, 8 aa, Homo sapiens
WLQLEMFA

SEQ ID NO: 62 - protein, 7 aa, Homo sapiens
LQLEMFA

SEQ ID NO: 63 - protein, 6 aa, Homo sapiens
QLEMFA

SEQ ID NO: 64 - protein, 5 aa, Homo sapiens
LEMFA

SEQ ID NO: 65 - protein, 4 aa, Homo sapiens
EMFA

SEQ ID NO: 66 - protein, 4 aa, Homo sapiens
DMFA

SEQ ID NO: 67 - protein, 5 aa, Homo sapiens
LEMFA

SEQ ID NO: 68 - protein, 5 aa, Homo sapiens
LDMFA

SEQ ID NO: 69 - protein, 700 aa, Homo sapiens
MAGIAAKLAKDREAAEGLGSHERAIKYLNQDYEALRNECLEAGTLFQDPSFPAIPSALGFKELGPYSSKTRGIEWKRPTEICADPQFIIGGATRTDICQGALGDCWLLAAIASLTLNEEILARVVPLNQSFQENYAGIFHFQFWQYGEWVEVVVDDRLPTKDGELLFVHSAEGSEFWSALLEKAYAKINGCYEALSGGATTEGFEDFTGGIAEWYELKKPPPNLFKIIQKALQKGSLLGCSIDITSAADSEAITFQKLVKGHAYSVTGAEEVESNGSLQKLIRIRNPWGEVEWTGRWNDNCPSWNTIDPEERERLTRRHEDGEFWMSFSDFLRHYSRLEICNLTPDTLTSDTYKKWKLTKMDGNWRRGSTAGGCRNYPNTFWMNPQYLIKLEEEDEDEEDGESGCTFLVGLIQKHRRRQRKMGEDMHTIGFGIYEVPEELSGQTNIHLSKNFFLTNRARERSDTFINLREVLNRFKLPPGEYILVPSTFEPNKDGDFCIRVFSEKKADYQAVDDEIEANLEEFDISEDDIDDGFRRLFAQLAGEDAEISAFELQTILRRVLAKRQDIKSDGFSIETCKIMVDMLDSDGSGKLGLKEFYILWTKIQKYQKIYREIDVDRSGTMNSYEMRKALEEAGFKMPCQLHQVIVARFADDQLIIDFDNFVRCLVRLETLFKIFKQLDPENTGTIELDLISWLCFSVL

SEQ ID NO: 70 - protein, 700 aa, Mus musculus
MAGIAIKLAKDREAAEGLGSHERAIKYLNQDYETLRNECLEAGALFQDPSFPALPSSLGYKELGPYSSKTRGIEWKRPTEICADPQFIIGGATRTDICQGALGDCWLLAAIASLTLNEEILARVVPPDQSFQENYAGIFHFQFWQYGEWVEVVVDDRLPTKDGELLFVHSAEGSEFWSALLEKAYAKINGCYETLSGGATTEGFEDFTGGIAEWYELRKPPPNLFKIIQKALEKGSLLGCSIDITSAADSEAVTYQKLVKGHAYSVTGAEEVESSGSLQKLIRIRNPWGQVEWTGKWNDNCPSWNTVDPEVRANLTERQEDGEFWMSFSDFLRHYSRLEICNLTPDTLTCDSYKKWKLTKMDGNWRRGSTAGGCRNYPNTFWMNPQYLIKLEEEDEDEEDGGRGCTFLVGLIQKHRRRQRKMGEDMHTIGFGIYEVPEELTGQTNIHLGKNFFLTTRARERSDTFINLREVLNRFKLPPGEYVLVPSTFEPHKDGDFCIRVFSEKKADYQAVDDEIEANIEEIDANEEDIDDGFRRLFVQLAGEDAEISAFELQTILRRVLAKRQDIKSDGFSIETCKIMVDMLDEDGSGKLGLKEFYILWTKIQKYQKIYREIDVDRSGTMNSYEMRKALEEAGFKLPCQLHQVIVARFADDELIIDFDNFVRCLVRLETLFKIFKQLDPENTGTIQLNLASWLSFSVL

SEQ ID NO: 71 - protein, 700 aa, Sus scrofa
MAGIAAKLAKDREAAEGLGSHERAVKYLNQDYAELRDQCLEAGALFQDPSFPALPSSLGFKELGPYSGKTRGIEWKRPTEICDNPQFIIGGATRTDICQGALGDCWLLAAIASLTLNEEVLARVVPLDQSFQENYAGIFRFQFWQYGEWVEVVVDDRLPTKDGELLFVHSAEGSEFWSALLEKAYAKINGCYEALSGGATTEGFEDFTGGIAEWYELRKAPPNLFKIIQKALQKGSLLGCSIDITSAADSEAVTFQKLVKGHAYSVTGAEEVESRGSLQKLIRIRNPWGEVEWTGQWNDNCPNWNTVDPEVRESLTRRHEDGEFWMSFSDFLRHYSRLEICNLTPDTLTSDSYKKWKLTKMDGNWRRGSTAGGCRNYPNTFWMNPQYLIKLEEEDEDQEDGESGCTFLVGLIQKHRRRQRKMGEDMHTIGFGIYEVPEELTGQTNIHLSKNFFLTHRARERSDTFINLREVLNRFKLPPGEYILVPSTFEPNKDGDFCIRVFSEKKADYQVVDDEIEADLEENDASEDDIDDGFRRLFAQLAGEDAEISAFELQTILRRVLAKRQDIKSDGFSIETCKIMVDMLDSDGSAKLGLKEFYILWTKIQKYQKIYREIDVDRSGTMNSYEMRKALEEAGFKLPCQLHQVIVARFADDQLIIDFDNFVRCLVRLETLFRISKQLDSENTGTIELDLISWLCFSVL

SEQ ID NO: 72 - protein, 700 aa, Rattus norvegicus [sequence omitted]

SEQ ID NO: 73 - protein, 700 aa, Bos taurus
MAGIAAKLAKDREAAEGLGSHERAVKYLNQDYAALRDECLEAGALFQDPSFPALPSSLGFKELGPYSSKTRGIEWKRPTEICDNPQFITGGATRTDICQGALGDCWLLAAIASLTLNEEILARVVPLDQSFQENYAGIFHFQFWQYGEWVEVVVDDRLPTKDGELLFVHSAEGSEFWSALLEKAYAKINGCYEALSGGATTEGFEDFTGGIAEWYELRKAPPNLFRIIQKALQKGSLLGCSIDITSAADSEAITFQKLVKGHAYSVTGAEEVESRGSLQKLIRIRNPWGEVEWTGQWNDNCPNWNTVDPEVRETLTRQHEDGEFWMSFNDFLRHYSRLEICNLTPDTLTSDSYKKWKLTKMDGNWRRGSTAGGCRNYPNTFWMNPQYLIKLEEEDEDQEDGESGCTFLVGLIQKHRRRQRKMGEDMHTIGFGIYEVPEELTGQTNIHLSKKFFLTTRARERSDTFINLREVLNRFKLPPGEYIVVPSTFEPNKDGDFCIRVFSEKKADYQVVDDEIEANIDEIDISEDDIDDGFRRLFAQLAGEDAEISAFELQTILRRVLAKRQDIKSDGFSIETCKIMVDMLDSDGSGKLGLKEFYILWTKIQKYQKIYREIDVDRSGTMNSYEMRKALEEAGFKMPCQLHQVIVARFADDDLIIDFDNFVRCLIRLETLFRIFKQLDPENTGMIQLDLISWLSFSVL

SEQ ID NO: 74 - protein, 24 aa, Homo sapiens
KQLDPENTGTIELDLISWLCFSVL

SEQ ID NO: 75 - protein, 23 aa, Homo sapiens
QLDPENTGTIELDLISWLCFSVL

SEQ ID NO: 76 - protein, 22 aa, Homo sapiens
LDPENTGTIELDLISWLCFSVL

SEQ ID NO: 77 - protein, 21 aa, Homo sapiens
DPENTGTIELDLISWLCFSVL

SEQ ID NO: 78 - protein, 20 aa, Homo sapiens
PENTGTIELDLISWLCFSVL

SEQ ID NO: 79 - protein, 19 aa, Homo sapiens
ENTGTIELDLISWLCFSVL

SEQ ID NO: 80 - protein, 18 aa, Homo sapiens
NTGTIELDLISWLCFSVL

SEQ ID NO: 81 - protein, 17 aa, Homo sapiens
TGTIELDLISWLCFSVL

SEQ ID NO: 82 - protein, 16 aa, Homo sapiens
GTIELDLISWLCFSVL

SEQ ID NO: 83 - protein, 15 aa, Homo sapiens
TIELDLISWLCFSVL

SEQ ID NO: 84 - protein, 14 aa, Homo sapiens
IELDLISWLCFSVL

SEQ ID NO: 85 - protein, 14 aa, Homo sapiens
IELDLISWLCFDVL

SEQ ID NO: 86 - protein, 14 aa, Homo sapiens
IELDLISWLCFEVL

SEQ ID NO: 87 - protein, 13 aa, Homo sapiens
ELDLISWLCFSVL

SEQ ID NO: 88 - protein, 12 aa, Homo sapiens
LDLISWLCFSVL

SEQ ID NO: 89 - protein, 11 aa, Homo sapiens
DLISWLCFSVL

SEQ ID NO: 90 - protein, 12 aa, Homo sapiens
LDLISWLCFDVL

SEQ ID NO: 91 - protein, 12 aa, Homo sapiens
LDLISWLCFEVL

SEQ ID NO: 92 - protein, 10 aa, Homo sapiens
LISWLCFSVL

SEQ ID NO: 93 - protein, 9 aa, Homo sapiens
ISWLCFSVL

SEQ ID NO: 94 - protein, 24 aa, Homo sapiens
KQLDPENTGTIELDLISWLCFDVL

SEQ ID NO: 95 - protein, 23 aa, Homo sapiens
QLDPENTGTIELDLISWLCFDVL

SEQ ID NO: 96 - protein, 22 aa, Homo sapiens
LDPENTGTIELDLISWLCFDVL

SEQ ID NO: 97 - protein, 21 aa, Homo sapiens
DPENTGTIELDLISWLCFDVL

SEQ ID NO: 98 - protein, 20 aa, Homo sapiens
PENTGTIELDLISWLCFDVL

SEQ ID NO: 99 - protein, 19 aa, Homo sapiens
ENTGTIELDLISWLCFDVL

SEQ ID NO: 100 - protein, 18 aa, Homo sapiens
NTGTIELDLISWLCFDVL

SEQ ID NO: 101 - protein, 17 aa, Homo sapiens
TGTIELDLISWLCFDVL

SEQ ID NO: 102 - protein, 16 aa, Homo sapiens
GTIELDLISWLCFDVL

SEQ ID NO: 103 - protein, 15 aa, Homo sapiens
TIELDLISWLCFDVL

SEQ ID NO: 104 - protein, 14 aa, Homo sapiens
IELDLISWLCFDVL

SEQ ID NO: 105 - protein, 14 aa, Homo sapiens
IELDLISWLCFDVL

SEQ ID NO: 106 - protein, 13 aa, Homo sapiens
ELDLISWLCFDVL

SEQ ID NO: 107 - protein, 12 aa, Homo sapiens
LDLISWLCFDVL

SEQ ID NO: 108 - protein, 11 aa, Homo sapiens
DLISWLCFDVL

SEQ ID NO: 109 - protein, 24 aa, Homo sapiens
KQLDPENTGTIELDLISWLCFEVL

SEQ ID NO: 110 - protein, 23 aa, Homo sapiens
QLDPENTGTIELDLISWLCFEVL

SEQ ID NO: 111 - protein, 22 aa, Homo sapiens
LDPENTGTIELDLISWLCFEVL

SEQ ID NO: 112 - protein, 21 aa, Homo sapiens
DPENTGTIELDLISWLCFEVL

SEQ ID NO: 113 - protein, 20 aa, Homo sapiens
PENTGTIELDLISWLCFEVL

SEQ ID NO: 114 - protein, 19 aa, Homo sapiens
ENTGTIELDLISWLCFEVL

SEQ ID NO: 115 - protein, 18 aa, Homo sapiens
NTGTIELDLISWLCFEVL

SEQ ID NO: 116 - protein, 17 aa, Homo sapiens
TGTIELDLISWLCFDVL

SEQ ID NO: 117 - protein, 16 aa, Homo sapiens
GTIELDLISWLCFEVL

SEQ ID NO: 118 - protein, 15 aa, Homo sapiens
TIELDLISWLCFEVL

SEQ ID NO: 119 - protein, 14 aa, Homo sapiens
IELDLISWLCFEVL

SEQ ID NO: 120 - protein, 14 aa, Homo sapiens
IELDLISWLCFEVL

SEQ ID NO: 121 - protein, 13 aa, Homo sapiens
ELDLISWLCFDVL

SEQ ID NO: 122 - protein, 12 aa, Homo sapiens
LDLISWLCFDVL

SEQ ID NO: 123 - protein, 11 aa, Homo sapiens
DLISWLCFDVL

SEQ ID NO: 124 - protein, 10 aa, Homo sapiens
LISWLCFSVL

SEQ ID NO: 125 - protein, 9 aa, Homo sapiens
ISWLCFSVL

SEQ ID NO: 126 - protein, 9 aa, Homo sapiens
ISWLCFDVL

SEQ ID NO: 127 - protein, 9 aa, Homo sapiens
ISWLCFEVL

SEQ ID NO: 128 - protein, 8 aa, Homo sapiens
SWLCFSVL

SEQ ID NO: 129 - protein, 8 aa, Homo sapiens
SWLCFDVL

SEQ ID NO: 130 - protein, 8 aa, Homo sapiens
SWLCFEVL

SEQ ID NO: 131 - protein, 7 aa, Homo sapiens
WLCFSVL

SEQ ID NO: 132 - protein, 9 aa, Homo sapiens
ISWLCFDVL

SEQ ID NO: 133 - protein, 9 aa, Homo sapiens
ISWLCFEVL

SEQ ID NO: 134 - protein, 8 aa, Homo sapiens
SWLCFSVL

SEQ ID NO: 135 - protein, 8 aa, Homo sapiens
SWLCFDVL

SEQ ID NO: 136 - protein, 8 aa, Homo sapiens
SWLCFEVL

SEQ ID NO: 137 - protein, 7 aa, Homo sapiens
WLCFSVL

SEQ ID NO: 138 - protein, 6 aa, Homo sapiens
LCFDVL

SEQ ID NO: 139 - protein, 6 aa, Homo sapiens
LCFEVL

SEQ ID NO: 140 - protein, 5 aa, Homo sapiens
CFSVL

SEQ ID NO: 141 - protein, 5 aa, Homo sapiens
CFDVL

SEQ ID NO: 142 - protein, 5 aa, Homo sapiens
CFEVL

SEQ ID NO: 143 - protein, 4 aa, Homo sapiens
FSVL

SEQ ID NO: 144 - protein, 4 aa, Homo sapiens
FDVL

SEQ ID NO: 145 - protein, 4 aa, Homo sapiens
FEVL

SEQ ID NO: 146 - protein, 24 aa, Homo sapiens
IPSQRRYVYYYSYLLKNHLDYRPV

SEQ ID NO: 147 - protein, 22 aa, Homo sapiens
PSQRRYVYYYSYLLKNHLDYRP

SEQ ID NO: 148 - protein, 22 aa, Homo sapiens
PSQRRYVYYYSYLLKNHLDYRP

SEQ ID NO: 149 - protein, 21 aa, Homo sapiens
PSQRRYVYYYSYLLKNHLDYR

SEQ ID NO: 150 - protein, 20 aa, Homo sapiens
PSQRRYVYYYSYLLKNHLDY

SEQ ID NO: 151 - protein, 18 aa, Homo sapiens
SQRRYVYYYSYLLKNHLD

SEQ ID NO: 152 - protein, 19 aa, Homo sapiens
PSQRRYVYYYSYLLKNHLD

SEQ ID NO: 153 - protein, 18 aa, Homo sapiens
PSQRRYVYYYSYLLKNHL

SEQ ID NO: 154 - protein, 17 aa, Homo sapiens
PSQRRYVYYYSYLLKNH

SEQ ID NO: 155 - protein, 16 aa, Homo sapiens
PSQRRYVYYYSYLLKN

SEQ ID NO: 156 - protein, 15 aa, Homo sapiens
PSQRRYVYYYSYLLK

SEQ ID NO: 157 - protein, 17 aa, Homo sapiens
SQRRYVYYYSYLLKNHL

SEQ ID NO: 158 - protein, 16 aa, Homo sapiens
QRRYVYYYSYLLKNHL

SEQ ID NO: 159 - protein, 15 aa, Homo sapiens
QRRYVYYYSYLLKNH

SEQ ID NO: 160 - protein, 14 aa, Homo sapiens
QRRYVYYYSYLLKN

SEQ ID NO: 161 - protein, 13 aa, Homo sapiens
QRRYVYYYSYLLK

SEQ ID NO: 162 - protein, 12 aa, Homo sapiens
RRYVYYYSYLLK

SEQ ID NO: 163 - protein, 18 aa, Homo sapiens
QRRYVYYYSYLLKNHLDY

SEQ ID NO: 164 - protein, 17 aa, Homo sapiens
RRYVYYYSYLLKNHLDY

SEQ ID NO: 165 - protein, 15 aa, Homo sapiens
YVYYYSYLLKNHLDY

SEQ ID NO: 166 - protein, 13 aa, Homo sapiens
YYYSYLLKNHLDY

SEQ ID NO: 167 - protein, 12 aa, Homo sapiens
YYSYLLKNHLDY

SEQ ID NO: 168 - protein, 11 aa, Homo sapiens
YSYLLKNHLDY

SEQ ID NO: 169 - protein, 10 aa, Homo sapiens
SYLLKNHLDY

SEQ ID NO: 170 - protein, 9 aa, Homo sapiens
YLLKNHLDY

SEQ ID NO: 171 - protein, 8 aa, Homo sapiens
LLKNHLDY

SEQ ID NO: 172 - protein, 11 aa, Homo sapiens
YYSYLLKNHLD

SEQ ID NO: 173 - protein, 9 aa, Homo sapiens
YSYLLKNHL

SEQ ID NO: 174 - protein, 7 aa, Homo sapiens
SYLLKNH

SEQ ID NO: 175 - protein, 5 aa, Homo sapiens
YLLKN

SEQ ID NO: 176 - protein, 8 aa, Homo sapiens
YLLKNHLD

SEQ ID NO: 177 - protein, 4 aa, Homo sapiens
YLLK

SEQ ID NO: 178 - protein, 4 aa, Homo sapiens
LLKN

SEQ ID NO: 179 - protein, 52 aa, Homo sapiens
ERADNDKEYLVLTLTKNDLDKANKDKANRYFSPNFKVKLYFTKTVEEPSNPE

SEQ ID NO: 180 - protein, 25 aa, Homo sapiens
NRYFSPNFKVKLYFTKTVEEPSNPE

SEQ ID NO: 181 - protein, 41 aa, Homo sapiens
KEYLVLTLTKNDLDKANKDKANRYFSPNFKVKLYFTKTVEE

SEQ ID NO: 182 - protein, 25 aa, Homo sapiens
ERADNDKEYLVLTLTKNDLDKANKD

SEQ ID NO: 183 - protein, 12 aa, Homo sapiens
KEYLVLTLTKND

SEQ ID NO: 184 - protein, 10 aa, Homo sapiens
EYLVLTLTKN

SEQ ID NO: 185 - protein, 9 aa, Homo sapiens
EYLVLTLTK

SEQ ID NO: 186 - protein, 10 aa, Homo sapiens
KEYLVLTLTK

SEQ ID NO: 187 - protein, 8 aa, Homo sapiens
YLVLTLTK

SEQ ID NO: 188 - protein, 6 aa, Homo sapiens
LVLTLT

SEQ ID NO: 189 - protein, 5 aa, Homo sapiens
VLTLT

SEQ ID NO: 190 - protein, 4 aa, Homo sapiens
VLTL

SEQ ID NO: 191 - protein, 4 aa, Homo sapiens
LTLT

SEQ ID NO: 192 - protein, 5 aa, Homo sapiens
EYLVL

SEQ ID NO: 193 - protein, 4 aa, Homo sapiens
EYLV

SEQ ID NO: 194 - protein, 7 aa, Artificial Sequence
  7-mer
RRMKWKK

SEQ ID NO: 195 - protein, 33 aa, Artificial Sequence
  Transportan
GWTLNSAGYLLGKINLKALAALAKISILAMIDE

SEQ ID NO: 196 - protein, 16 aa, Artificial Sequence
  Penatrin
RQIKIWFQNRRMKWKK

SEQ ID NO: 197 - protein, 10 aa, Artificial Sequence
  PolyArginine
RRRRRRRRRR

SEQ ID NO: 198 - protein, 18 aa, Artificial Sequence
  MAP
LLIILRRRIRKQAHAHSK

SEQ ID NO: 199 - protein, 39 aa, Artificial Sequence
  RDP
KSVRTWNEIIPSKGCLRVGGRCHPHVNGGGRRRRRRRRR

SEQ ID NO: 200 - protein, 9 aa, Human immunodeficiency virus
RKKRRQRRR

SEQ ID NO: 201 - protein, 24 aa, Homo sapiens
NRYFSPNFKVKLYFTKTVEEPSNP

SEQ ID NO: 202 - protein, 23 aa, Homo sapiens
RYFSPNFKVKLYFTKTVEEPSNP

SEQ ID NO: 203 - protein, 22 aa, Homo sapiens
RYFSPNFKVKLYFTKTVEEPSN

SEQ ID NO: 204 - protein, 21 aa, Homo sapiens
YFSPNFKVKLYFTKTVEEPSN

SEQ ID NO: 205 - protein, 20 aa, Homo sapiens
YFSPNFKVKLYFTKTVEEPS

SEQ ID NO: 206 - protein, 19 aa, Homo sapiens
FSPNFKVKLYFTKTVEEPS

SEQ ID NO: 207 - protein, 18 aa, Homo sapiens
FSPNFKVKLYFTKTVEEP

SEQ ID NO: 208 - protein, 17 aa, Homo sapiens
SPNFKVKLYFTKTVEEP

SEQ ID NO: 209 - protein, 16 aa, Homo sapiens
SPNFKVKLYFTKTVEE

SEQ ID NO: 210 - protein, 15 aa, Homo sapiens
PNFKVKLYFTKTVEE

SEQ ID NO: 211 - protein, 14 aa, Homo sapiens
PNFKVKLYFTKTVE

SEQ ID NO: 212 - protein, 13 aa, Homo sapiens
NFKVKLYFTKTVE

SEQ ID NO: 213 - protein, 12 aa, Homo sapiens
NFKVKLYFTKTV

SEQ ID NO: 214 - protein, 11 aa, Homo sapiens
FKVKLYFTKTV

SEQ ID NO: 215 - protein, 10 aa, Homo sapiens
FKVKLYFTKT

SEQ ID NO: 216 - protein, 9 aa, Homo sapiens
KVKLYFTKT

SEQ ID NO: 217 - protein, 8 aa, Homo sapiens
KVKLYFTK

SEQ ID NO: 218 - protein, 7 aa, Homo sapiens
VKLYFTK

SEQ ID NO: 219 - protein, 6 aa, Homo sapiens
VKLYFT

SEQ ID NO: 220 - protein, 5 aa, Homo sapiens
KLYFT

SEQ ID NO: 221 - protein, 4 aa, Homo sapiens
KLYF